(19) United States
(12) Patent Application Publication
(10) Pub. No.: US 2025/0051793 A1
(43) Pub. Date: Feb. 13, 2025

(54) IMPROVED SCREENING METHOD FOR GENOME EDITED EVENTS

(71) Applicant: BASF Agricultural Solutions Seed US LLC, Florham Park, NJ (US)

(72) Inventors: Timothy James GOLDS, Gent (BE); David DE VLEESSCHAUWER, Gent (BE); Eveline BOSSIER, Gent (BE); Katelijn D'HALLUIN, Gent (BE); Frank MEULEWAETER, Gent (BE)

(21) Appl. No.: 18/723,266

(22) PCT Filed: Dec. 20, 2022
(86) PCT No.: PCT/EP2022/086982

§ 371 Date: Jun. 21, 2024

(30) Foreign Application Priority Data
Dec. 22, 2021 (EP) 21216795.1

(51) Int. Cl. C12N 15/82; C12N 5/04; C12Q 1/6895

(52) U.S. Cl. CPC C12N 15/8243; C12N 5/04; C12Q 1/6895; C12Q 2600/13

(57) ABSTRACT

The present invention is in the field of plant molecular biology and is directed to a method for improved screening for known edits within the genome of a cell. The method of the invention comprises dividing a population of plant cells comprising a desired nucleic acid sequence, into subgroups, quantifying the concentration of the desired nucleic acid sequence for each sub-group, culturing cells from the a sub-group(s), dividing the cells into subgroups, quantifying the concentration of the desired nucleic acid sequence in each subgroup, and regenerating intact individual plants from the cells of a selected subgroup(s) with the desired nucleic acid sequence.

### DESCRIPTION OF THE INVENTION

The present invention is in the field of molecular biology, especially plant molecular biology and is directed to a method for improved screening for known edits within the genome of a cell, e.g., as a direct consequence of genome editing.

### INTRODUCTION

Cells carrying a specific genomic mutation can be difficult to detect and enrich if they do not contain a marker that allows for direct detection of the cells. Such mutations can occur naturally or spontaneously, for example during the growth and breeding process, as a result of exogenous or endogenous DNA damage, or due to genetic methods or genome editing processes.

Genome editing technologies including the introducing of precise gene edits is well documented in plants. Methods well established in the art introduce a double strand DNA break (DSB) in the genome using technologies such as Zn-finger nucleases, homing endonucleases, TALENs or RNA guided nucleases e.g., CRISPR systems, for example Casλ, or CRISPR Cas systems, e.g. CRISPR CAS9 or CRISPR Cas12a.

Genome editing applied to plant cells is reasonably efficient leading to mutations comprising targeted insertions and/or deletions (InDels), if the double strand break (DSB) in the genome is repaired by the error-prone non-homologous end joining pathway (NHEJ), or to unaltered genomic sequences, where the editing approach is unsuccessful. If the DSB is repaired by homologous recombination and a repair or donor template, a precise edit (PE) can be obtained, although at a much lower frequency.

Although the efficiency of the production of cells comprising targeted edits has been improved since the development of CRISPR methods, as compared to the use of homing endonucleases or TALENs, it is still a relatively rare event in eukaryotic cells, especially where no selectable marker gene is introduced that enables direct selection/enrichment of the mutated or edited cells.

Zhang et al (Zhang et al, 2016, Nat. Commun. 7:12617) describe two genome-editing methods in which CRISPR/Cas9 is introduced as DNA or RNA into plant cells and expressed transiently, thus generating edited genes with a transgene-free protocol. The procedure avoids antibiotic/herbicide selection. After the CRISPR/Cas9 DNA or RNA is introduced into wheat immature embryos by particle bombardment and transiently expressed, callus cells are produced, and plants are regenerated. The TO plants are examined by PCR-RE and DNA sequencing to identify targeted mutants. The absence of an herbicide selection step supported the generation of transgene-free mutants even after bombarded with a CRISPR/Cas9 DNA. Plants with targeted mutations and lacking active transgenes were obtained with both methods, the transiently expressed CRISPR/Cas9 DNA as well as with CRISPR/Cas9 RNA. Also, Liang et al. (Liang et al., 2018, Nature Protocols, Vol. 13, No. 2, p.413f) describe a DNA-free editing method in which ribonucleoproteins of CRISPR/Cas9 (RNPs) are delivered into the target cells by particle bombardment. The protocol allows the generation and identification of edited plants, within nine to eleven weeks, without the use of any foreign DNA and without an antibiotic/herbicide selection step.

Both Zhang et al (Zhang et al, 2016, Nat. Commun. 7:12617) and Liang et al. (Liang et al., 2018, Nature Protocols, Vol. 13, No. 2, p.413f) utilize a mutant screening method using pools of shoots. Pieces of labelled leaf samples of three to four shoots generated from the same bombarded embryo are pooled and then checked by PCR-RE for the editing events. Shoots in a pool with a positive PCR-RE signal are grown for several further days if necessary and then checked individually by PCR-RE and confirmed by sequencing. However, relatively low mutagenesis frequencies were observed in the analyzed plants.

Also, WO2018/001884 provides a method that supports the preparation, selection and propagation of an organism that comprises a specific, predetermined mutation by a pooling and splitting approach because the statistical probability is extremely low when it comes to finding a predetermined nucleotide substitution. WO2018/001884 describes a method in which a pool of mutated organisms is first divided into sub-pools while the potential for reproduction of organisms from each genotype within said analyzed pool is maintained, and then tested by PCR, e.g., ddPCR, for the presence of the mutation. The organisms of the positive pools are then subsequently divided into a secondary pool, which then is tested by PCR, e.g., ddPCR, again for the presence of the mutation in each genotype within the pool, again while the potential for reproduction of organisms from each genotype within said analyzed pool is maintained. Accordingly, the method requires that each genotype is kept within each sub-pool such that the potential for reproduction of organisms from each genotype within said analyzed pool is maintained. Thus, the method comprises for example a repeated propagation of all present genotypes followed by an analysis of at least one copy of each genotypes, aiming to maintain another copy of each genotype in the population. Accordingly, the population produced by the method of WO2018/001884 comprises a copy of the mutagenized genotypes while its existence is identified after a propagation step via a digital PCR-based system.

The methods disclosed in WO2018001884, Zhang et al. and Liang et al. for the identification of positive clones are costly and cumbersome.

There was, thus, still a need in the art for a method that allows efficiently and rapidly identifying, after a mutagenizing step, those cells that comprise a desired mutation, e.g., a mutation at a target site. Needed is a method that allows the identification of those organisms in a large population of organisms that show a desired nucleotide acid sequence in a known region (positive event).

### DETAILED DESCRIPTION OF THE INVENTION

Accordingly, the present invention relates to a three-step method for producing plants comprising a desired nucleic acid sequence that method comprises or, preferably consists the following selection steps:
1. Dividing a population of cells comprising regenerative cells, into subgroups, testing a sample from each subgroup to quantify the concentration of the desired nucleic acid sequence,
2. Culturing the cells from the subgroup(s) with the highest concentration of the desired nucleic acid sequence, as tested in a sample of the subgroup, dividing the cultured cells, e.g. recovered calli or shoots, into subgroups, and dividing the cultured cells into subgroups and testing a sample from said subgroups for the concentration of the desired nucleic acids sequence; and
3. Regenerating plants from the cultured cells, e.g. calli or shoots, preferably generating plants from calli or shoots of a subgroup with cells having the highest concentration of the desired nucleic acid sequence, and testing the said plants for the presence of the desired nucleic acid sequence, selecting a plant from said plants tested, whereby the selected plant comprises the desired of nucleic acid sequence.

The term “desired nucleic acid sequence” shall mean a sequence of a nucleic acid molecule within an organism that is known and that is searched as present in the method of the invention. Such a desired sequence can be the result of a desired modification of a natural, non-desired nucleic acid molecule or a preferred natural version of a nucleic acid sequence that exists in multiple variations. A desired modification can for example be a variation or a modification of the sequence compared to a control or a wild type. The modification can be a preferred natural variation of a sequence or the result of a genome editing process or of a mutagenesis procedure.

The term “desired nucleic acid sequence” shall also comprise “desired nucleic acid sequences”. The term “desired nucleic acid sequence” shall mean that one nucleic acid molecule more than one sequence that is known and is searched for. The term “desired nucleic acid sequence” shall also mean that more than one nucleic acid molecules have a sequence that is known and is searched for. Consequently, the term “desired nucleic acid sequence” also comprises that multiple modifications on one or several nucleic acid molecules form the desired nucleic acid sequences. Thus, a desired nucleic acid sequence can also comprise several modifications in different location of a nucleic acid molecule, and/or several modifications in different nucleic acid molecules, e.g., modifications in a genome.

For example, a profile of modifications can be found on more than one molecule of a genome. For example, the modifications that define the “desired nucleic acid sequence” are found in one or more alleles, in one or more genes, or on one or more chromosomes of an organism. Preferably, the presence of the modification, a part thereof or all can be identified by a rapid and reliable method as described herein, e.g., a quantitative PCR method, preferably ddPCR (Droplet Digital PCR), or a high through-put sequencing method, like next generation sequencing (NGS). The term “subgroup with the highest concentration of the “desired nucleic acid sequence” means that in the corresponding subgroup the concentration of the desired nucleic acid sequence is higher than in other subgroups, preferably the highest.

The concentration of the desired nucleic acid sequence can be determined, for example, by measuring or determining the number of copies of the desired nucleic acid sequences in a sample. The concentration can be determined for example per volume, amount or other aliquot of a method for quantification, e.g. a ddPCR reaction. To the person skilled in the art there are other methods known that will allow an accurate quantification of the desired nucleic acid sequence in a sample.

Accordingly, the present invention comprises in one embodiment a method for the production of plants comprising a desired nucleic acid sequence, the method of the invention comprises the steps of:
a) Dividing a population of plant cells comprising regenerative plant cells, that comprises a subpopulation of cells comprising a desired nucleic acid sequence, into subgroups, quantifying the concentration of the desired nucleic acid sequence for each sub-group, each subgroup representing a subset of the populations genotype, and identifying one or more subgroups with the highest concentration of the desired nucleic acid sequence,
b) Culturing cells from the one or more sub-group(s) with the highest concentration of the desired nucleic acid sequence, dividing the cells into subgroups, quantifying the concentration of the desired nucleic acid sequence in each subgroup, each subgroup representing a subset of the population's genotype, and selecting one or more subgroups with the highest concentration of the desired nucleic acid sequence, and
c) Regenerating intact individual plants from the cells of one or more selected subgroup(s) of step (c), with the desired nucleic acid sequence.

In contrast, to the prior art methods, the method of the present invention does not require that the cells that are tested are treated such that the potential for reproduction of organisms from each genotype within said analyzed pool is maintained. It was found that is sufficient to determine the concentration of the desired sequence even if the genotype pool is amended as result of the examination. Thus, according to the present invention, each sub-group and each sample does not represent all genotypes present in the pool.

The regenerative cells used in the method of the invention are individual plant cells or cells within plant explants. In the method of the present invention, basically all explant types that are capable of generating dividing cells, to generate multicellular calli, capable of plant regeneration, can be used.

According to the present invention, a subgroup of cells identified in step (a) as comprising the appropriate high concentration of the desired nucleic acid sequence is selected and the cells are cultured to proliferate the cells and. depending on the regenerative cells used in the method of the invention, are regenerating.

Thus, according to the method of the invention, “culturing cells” means that the cultured cells are kept at conditions that allows proliferation of the cells and, preferably, starting a regenerative process to generate tissues from which shoots and plants can be recovered. Accordingly, “culturing cells” comprises that the cultured cells are proliferating. The step of “culturing cells” can comprise a morphogenic development of the cells to form, for example somatic embryos or shoots, e.g. from the cells of the subgroups selected in step (a). Accordingly, “culturing cells” comprises that the cultured cells can regenerate into calli or shoots. For example, “culturing cells” can comprise a process of proliferating cells, e.g. of the selected cells, and enriching the cells, e.g. calli. Thus, in one embodiment of the present invention, the cultured cells are calli that divide and are enriched.

Thus, in one preferred embodiment, dependent on the regenerative cells used in the method of the invention, “culturing cells” in step (b) of the method of the invention means that the step of culturing the cells selected in step (a) are regenerating to generate plant tissue or plants.

The plant cells, comprising regenerative cells, are, for example, selected from the group consisting of
single cells e.g., protoplasts or microspores,
cell aggregates e.g., cell suspensions or callus cultures,
complex multicellular explants from mature or immature seeds e.g., immature embryos, scutella or cotyledons,
complex multicellular explants from seedlings e.g., roots, hypocotyls, cotyledons, leaves, petioles or meristems, and
complex multicellular explants from plants e.g., roots, leaves, leaf-bases, petioles, stems, or meristems.

Thus, preferable, the cells are selected form the group consisting of protoplasts, microspores, cell suspensions, callus cultures, immature embryos, scutella, cotyledons, roots, leaves, leaf-bases, petioles, stems, meristems, roots, leaves, leaf-bases, petioles, stems, and meristems.

The plant cells, comprising regenerative cells, used in the method of the invention are not limited to these examples.

There is a huge interest to develop an efficient method for recovering and growing plants derived from genetically modified cells. Often, a selection of those cells comprising the desired nucleic acid sequence at a target position is only possible by analyzing the nucleic acid sequence at the desired position, e.g., via PCR or nucleic acid sequencing. These methods require the extraction and isolation of the RNA and/or DNA of the modified cells, a process that destroys the relevant cells. Methods known in the art require that the mutagenized cells must therefore first be cloned or proliferated before the presence of the desired nucleic acid sequence is analyzed. Accordingly, in the method of the prior art (WO2018001884), for saving or confirming the presence of each genotype in the surviving population that serves for the regeneration of the plants, the presence of the desired nucleic acid sequence is determined by a cell-destructive method for extracting the nucleic acid molecules of the cells, e.g., DNA and/or RNA, in a sample of a cloned or proliferated population of cells. Plants can be regenerated from the remaining population of cells that comprise cells with the genotype identified in the analysis.

However, according to the present invention, surprisingly, it was found that it is sufficient to take a sample of the population of cells without cloning or proliferating the cells. For example, it was surprisingly found that is it is possible to skip the cloning or proliferation step shown in the art, e.g., in WO2018001884. In the method of the present invention, it is not necessary to first clone the regenerative cells to ensure that a sufficient number of desired genetic modifications can be found in the regenerated plants or plant parts. Advantageously, an identification of the concentration of the desired nucleic acid sequence in a sample directly taken from the population of regenerative cells that shall comprise a desired nucleic acid sequence of, e.g., genome edited or genetically modified regenerative cells, allows an efficient production of plants carrying nucleic acid molecules with the desired nucleic acid sequence.

Thus, in one embodiment, the population of plant cells comprising regenerative cells, is first divided into subgroups and then, each subgroup is tested for the concentration of the desired nucleic acid sequence in said sub-group.

Accordingly, the method of the present invention allows, a much faster, more efficient, production of plants comprising a desired nucleic acid sequence from regenerative cells, as it works without the time-consuming steps of cloning and proliferating the genetically modified, e.g., mutagenized, engineered, or edited, regenerative cells.

In one embodiment, the plant cells comprising regenerative cells allow the regeneration of plants.

In one embodiment, the plant cells comprising regenerative cells comprise regenerative cells that are genetically modified. The method of the invention, thus, comprises, in one embodiment, genetically modifying the nucleic acid molecules of the cells before the concentration of the desired nucleic acid sequence is determined in subgroups of the modified cells.

Accordingly, in one embodiment, the method of the present invention comprises a step in which the nucleic acid molecules or genome of the plant cells comprising regenerative cells are chemically mutated or ionized, gene or genome edited, or genetically engineered before the cells are divided into sub-groups. In another embodiment, the plant cells comprising regenerative cells are genetically modified, e.g., as described, herein after the sub-groups of the regenerative cells have been formed. For example, the genetic modification takes place in each sub-group separately.

A “genetically modified” cell relates to a cell that was target of a treatment to amend or change its nucleic acids sequence, e.g., modifying one or more bases in its genome or modifying the sequence of the nucleic acids molecules of the genome, resulting in modification of the cell's DNA. The term “genetically modifying” a cell relates to a method to achieve a genetic modification in one or more cells. According to this invention, the nucleic acid sequence or the genome of a cell can be genetically modified by, for example, mutagenesis via treatment with chemicals or by radiation, oligonucleotide-directed mutagenesis (ODM), RNA interference, recombinant DNA methods or genome or gene engineering or editing, for example using site-directed nucleases (SDN). Thus, the term “genetically modified cell” shall also mean “mutated cell” or a “mutagenized cell” or “genome edited” cell. The term “genetically modified” shall also comprise a modification or mutation of the nucleic acid sequence or genome of a cell at a predefined site. Oligonucleotide-directed mutagenesis (ODM) allows the modification of a cell's nucleic acid sequence, e.g., the genome, without generating mutations in a non-predictable way.

“Genetic modification” also results from methods in which a cell is transformed or transfected. In a transformation or transfection of a cell additional nucleic acid molecules are introduced, parts of the genome are deleted, or a change in the genome's sequence is achieved. Recombinant DNA methods comprise inserting selected nucleic acid molecules into the genome of a cell or deleting a sequence, e.g., via homologous recombination. Preferably, the regenerative cells are modified by genome or gene edited. Basically, all targeted genome or gene editing methods can be used, ranging from NHEJ to HDR, e.g., base editing, prime editing, targeted random mutation technologies, etc.

In one preferred embodiment, the method to genetically modify plant cells is a targeted genome modification or mutagenesis, e.g., a mutation at one or more target sites, and includes but is not limited to single site mutations and targeted random mutation. In one embodiment, the genetic modification is an insertion or deletion of one or more nucleic acids more, preferably an InDel or point mutation.

Accordingly, the present invention comprises in one embodiment a step:
performing a targeted genetical modification of the plant cells comprising regenerative plant cells before dividing a population of plant cells comprising regenerative plant cells into subgroups and quantifying the concentration of the desired nucleic acid sequence for each sub-group.

The targeted modification can also be performed after the population of plant cells comprising regenerative plant cells is divided into subgroups. Accordingly, the present invention comprises in another embodiment a step:
performing a targeted genetical modification of plant cells comprising regenerative plant cells after dividing a population of plant cells comprising regenerative plant cells into subgroups and quantifying the concentration of the desired nucleic acid sequence for each sub-group.

Methods for the introduction of a mutation at a predefined site of a nucleic acid molecule in a cell are well known to the person skilled in the art and comprise for example, methods that are able to introduce targeted mutations, e.g., via the use of mega-nucleases, homing endonucleases, Zinc finger proteins, TALENs, Oligos (e.g., PNA, DNA, etc) and others. In one embodiment, the mutation of the nucleic acid sequence of the cells used in the method of the invention, e.g., to produce calli, shoots or plants, relates to base editing or prime editing, for example, using the CRISPR/Cas system. Accordingly, in one embodiment, the method comprises a step, wherein the mutation at the target site has been introduced using CRISPR, e.g., a Cas9 or Cas12a-system.

The mutations can be produced by introducing the corresponding enzymes (CRISPR components) into the cells, e.g., ribonucleoproteins (RNPs) or by stably or transiently transforming the cell with one or more nucleic acid molecules encoding the corresponding proteins or enzymes or nucleic acid molecules (for example gRNA) or mediating the expression of the corresponding molecules or a mixture thereof. Accordingly, the method of the present invention comprises introducing a mutation at a predefined site in the genome.

In one embodiment, the method comprises a step wherein random mutations, preferably targeted random mutations, are introduced at or near the target site. Methods for creating allelic diversity are known to the person skilled in the art and comprise for example, methods based on site-specific recruitment of mutagenic proteins such as error-prone DNA polymerases or highly active DNA deaminases. The mutagenic proteins can be delivered sequence-specifically to a target site either by protein fusion or non-covalent interaction with, for example, a programmable DNA binding domain and DNA endonuclease, e.g., a CRISPR-Cas effector protein. Accordingly, in one embodiment, the method comprises a step where DNA nucleotides within defined genomic windows are altered by means of CRISPR-related cytosine and/or adenine base editors. Adenine and cytosine base editors catalyse targeted A⋅T-to-G⋅C or C⋅G-to-T⋅A base pair changes, respectively. Cytosine base editors that can be used for targeted mutagenesis include Targeted AID-mediated mutagenesis (Ma et al., 2016), CRISPR-X (Hess et al., 2016) and Sun-Tag-based hypermutators (Rees and Liu., 2018). Dual cytosine and adenine base editor systems include STEME (Li et al., 2020), A&C-BEmax (Zhang et al., 2020) and SPACE (Grünewald et al., 2020). Other methods to introduce targeted random mutations include EvolvR and TRACE or similar: Halperin, S. O., et al., Nature 560, 248-252 (2018), Grünewald, J., et al. Nat Biotechnol 38, 861-864 (2020), Rees, H. A., and Liu, D. R. (2018). Nature reviews. Genetics, 19 (12), 770-788, Chen H, et al., Nat Biotechnol. 2020 February; 38 (2): 165-168, Zhang, X., et al. Nat Biotechnol 38, 856-860 (2020), Hess G T, et al., Nat Methods. 2016 December; 13 (12): 1036-1042Ma, Y., et al., Nat Methods 13, 1029-1035 (2016), Li, C., et al. Nat Biotechnol 38, 875-882 (2020).

The method to produce a mutated cell used in the present invention can comprise a step describing the delivery of DNA or proteins into the cell, for example, bombardment in a biolistic delivery system, or Agrobacterium-mediated transformation. The method, for example, comprises a step of single cell transformation of regenerative cells, for example multicellular explants, e.g., immature embryo explants, or single cells, e.g., protoplasts or microspores. Further target cell types are mentioned herein and are known to the person skilled in the art. The step of bombardment in a biolistic delivery system or Agrobacterium-mediated transformation can be performed before the subgroups are formed or thereafter. Corresponding methods are known in the art or described herein.

The method of the present invention is particular helpful if a phenotypic or agronomic difference between a genetically modified cell, or the derived plant or a part thereof, for example, between a cell which comprises the desired nucleic acid sequence in the targeted region and cell or a plant or part derived thereof and that comprises a wild type or undesired nucleic acid sequence can only be detected with difficulties, for example in a late development or growth stage. Visualization may also be dependent in some cases on number of targeted genes in the plant. Advantageously, the molecular screen method of the invention is independent of any agronomic or phenotypic difference.

Further, the present invention allows a fast screen to identify cells and plants comprising the desired nucleic acid sequence without the need to select the cells or plants first via selectable maker gene expression and selection methods. The use of selection markers is well known to the person skilled in the art and examples are described in the prior art. The presence of a desired nucleotide acid sequence at a target region can often not be identified by selection with a selection marker, which provides a read-out for cells that have received DNA components and may have a higher chance of carrying mutations.

Furthermore, even if possible, it is often not desired to introduce additional nucleic acid molecules or sequences into the cell. Accordingly, the method of the present invention comprises that cells of interest are enriched without the use of an external or endogenous selectable marker, e.g., a fluorescent reporter such as GFP or BFP or an antibiotic or herbicide tolerance or resistance gene.

Accordingly, in one embodiment, for a relevant time during culturing, developing or growing, e.g., until at least 1, 3, 6 or 9 months after modification of the regenerative cells, or only after plants start to mature, or only if the plants grow in the field or only if they are grown in a particular environment, the presence of the desired nucleic acid sequence shows any detectable effect such that the presence of the desired nucleic acid sequence can be assumed. In some cases, the presence of the desired nucleic acid sequence can only be detected under specific biotic or non-biotic stress conditions, for example stress conditions like, e.g., drought, nitrogen deficiency, or chemical treatment. In this situation, the screen as in the method of the present invention allows an early and less time-consuming production of a plant comprising the desired nucleic acid sequence.

Accordingly, in one embodiment, the medium or the growing condition, in which the cells, tissues or plants are cultured is not selective for the presence of a desired nucleic acid sequence in the genome of the cells, or tissue or plants comprising the modified cells.

Thus, according to the present invention it is sufficient (i) to divide the modified cells, e.g. modified plant cells comprising regenerative plant cells, into sub-groups, (ii) to analyze the concentration of the desired nucleic acid sequence in a sample of each subgroup and (ii) to continue with the sub-group or the sub-groups with the highest concentration to produce a plant comprising cells comprising the desired nucleic acid sequence. The highest concentration of the desired nucleic acid sequence is preferably determined after modifying the plant cells comprising regenerative plant cells.

For each step of the method of the invention, the concentration of the desired nucleic acid sequence at the target position can be determined by a sensitive, quantitative, preferably, rapid method. Thus, in one embodiment, the method of the invention comprises that the concentration of the nucleic acid sequence in the genome of the genetically modified plant cells is determined in a molecular screen. A sample of a subgroup is tested for the concentration of the desired sequence. According to the present invention, the concentration of the nucleic acid sequence in the sample can be determined by a quantitative molecular analytic method, e.g., by a qPCR, preferably by ddPCR, allowing in the following sequential steps a selection of those subgroups that comprise cells carrying the desired mutation. The quantitative molecular analysis method is performed in a sample, taken from the subgroup.

Advantageously, the present invention allows to produce and test a faster high number of genetically modified regenerative cells, as the time-consuming and resource-binding step of proliferation or cloning of the regenerative cells after the modification step is not required. Accordingly, the method of the invention comprises that in step (a) of the present invention, the subgroups of the plant cells comprising the genetically modified regenerative cells are directly tested for the concentration of the desired nucleic acid sequence. Such a test occurs in a sample taken from the subgroup.

According to the invention, for each step of the method of the invention, the term “identifying one or more subgroups with the highest concentration of the presence of the desired nucleic acid sequence” thus means that the concentration of the desired nucleic acid sequence is determined in a sample of said subgroup. Preferably, the sample of said subgroup is representative for the subgroup.

Thus, in one preferred embodiment, the method of the present invention comprises a step of identifying one or more subgroups of plant cells comprising regenerative plant cells with the highest concentration or concentrations, respectively, of the desired nucleic acid sequence.

Accordingly, in one embodiment, the method of the invention comprises a step for extracting the nucleic acid molecules, e.g., DNA and/or RNA, from a sample of each subgroup of genetically modified cells and destroying the cells of the sample in the analysis of the concentration of the desired nucleic acid sequence in said sample, as described herein. For example, a sample of cells taken for the analysis in step (a) comprises preferably the minimal number of cells that gives an accurate prediction for the population of cells in the subgroup. In one embodiment, around 30% or 15% or less of the cells of a subgroup, preferably, less than, e.g., 12%, 10%, 7%, 5%, 3%, 1%, 0.5%, 0, 1% or less cells are used for DNA extraction. The number of cells collected in a sample of a subgroup and that are analyzed depends on the expected quantity of cells carrying the desired nucleic acid sequence.

For example, the efficiency of the genetic modification method, e.g., particle bombardment or Agrobacterium-mediated transformation, can determine the abundance of the desired nucleic acid sequence in the population. Thus, the method used to determine the concentration of the presence of the desired nucleic acid sequence in the DNA of the tested cells is sensitive enough to detect the frequency of the events.

As described herein, the concentration of the desired nucleic acid sequence in the sample can be determined by PCR-based analysis or nucleic acid sequencing of the relevant region, for example by qPCR, digital PCR, preferably, digital droplet PCR (ddPCR), or by high through-put sequencing methods, e.g., next generation sequencing (NGS). In one preferred embodiment, the concentration of the presence of the desired nucleic acid sequence at the target position in the genetically modified regenerative cells is determined by ddPCR. It was found that ddPCR can be used well for the determination of said frequency in a quick and reliable manner. ddPCR that can be used for example as described herein or as described, for example, in WO2018001884 or Peng C, et al (2020). Front. Plant Sci. 11:610790, or Miyaoka Y et al., (2018) Methods in Molecular Biology (Clifton, N.J.), 1768, 349-362.

Thus, in a preferred embodiment, the method of the invention comprises in the first step (a) genetically modifying, e.g., mutating, transforming or transfecting. the plant cells comprising regenerative plant cells as described herein, dividing said modified cells into subgroups and determining then the concentration of the desired nucleic acid sequence in each subgroup, preferably by a molecular screen, e.g., NGS or ddPCR.

In another embodiment, the method of the present invention comprises first dividing the plant cells comprising regenerative plant cells into subgroups, then modifying, e.g., mutating, transforming or transfecting, the cells in said sub-groups, and identifying the sub-group with the highest concentration of the desired nucleic acid sequence, preferably by a molecular screen, e.g., NGS or ddPCR. The concentration of the desired nucleic acid sequence is determined in a representative sample of the DNA of each subgroup.

Accordingly, no proliferation of the cells before or after genetically modifying the regenerative cells is necessary.

According to the method of the invention, the concentration of the presence of the desired nucleic acid sequence relates to the relative number of genomes, and, thus, cells that carry the desired nucleic acid sequence compared to the number of cells that do not carry the desired nucleic acid sequence.

According to the method of the invention, the identifying one or more subgroups with the highest concentration or concentrations, respectively, of the concentration of the desired nucleic acid sequence comprises that one or more samples of each subgroup is determined. For example, according to the present invention, the DNA is extracted from the cells to be tested, analyzed for the concentration of the desired nucleic acid sequence such that the one or more subgroups with the highest concentration(s) can be identified.

According to the method of the invention, the selection of one or more subgroups comprising plant cells comprising regenerative cells with the highest concentration or concentrations, respectively, of the desired nucleic acid sequence means that from all subgroups that were tested, an appropriate number of subgroups for the next step is selected.

The preferred selected number of subgroups depends, for example, from the number of cells in each subgroup, or the number of the subgroups and/or the expected or predicted concentration of the desired nucleic acid sequence in the population. For example, if the number of cells carrying the desired nucleic acid sequence is found to be low, the skilled person can select more subgroups than if the number is high.

The size of the subgroups, e.g., the number of cells per subgroup, is chosen by the person skilled in the art in an appropriate manner. The preferred number of cells per subgroup depends, for example, on the total number of the cells in the population, the efficiency of the modification process and/or the expected, predicted or from an analyzed sample predicted concentration of the desired nucleic acid sequence in the population.

Preferably, a subgroup is selected for the next step of the method of the invention, if the selected subgroup has a high concentration of desired nucleic acid sequences, e.g., higher than the average of all tested samples in the same step. Preferably, the concentrations of the nucleic acid sequence in the selected subgroups are the highest concentrations found in all subgroups.

In one embodiment, five, four, three, preferably two or one subgroup(s) with the highest concentrations of the desired nucleic acid sequence of all tested subgroups are selected for the next step. A small number of selected subgroups is preferred, e.g., only one or two subgroups are selected in a step.

In one embodiment, e.g., for the production of genetically modified plants, the modification efficiency is first determined, and the population of genetically modified cells comprising regenerative plant cells is divided into subgroups. In one embodiment, the efficiency of the genetic modification is not tested, and the population is divided before or after the genetic modification process was performed.

Accordingly, in one embodiment, the method of the invention comprises a step (a) comprising, preferably consisting of, the steps:
a) (i) providing a population of plant cells comprising regenerative plant cells expected to have a desired nucleic acid sequence, (ii) dividing the population of said plant cells into subgroups, (iii) extracting DNA and/or RNA from a sample from said cells of each subgroup, (iv) identifying one or more subgroups with the highest concentration of the desired nucleic acid sequence, and (v) selecting one or more subgroups of said cells with the highest concentration of the desired nucleic acid sequence, whereby, optionally, and in another embodiment preferably, said cells are plant cells comprising genetically modified regenerative plant cells.

Accordingly, in another embodiment, the method of the invention comprises a step (a) comprising, preferably consisting of, the steps:
a) (i) dividing the population plant cells comprising regenerative plant cells into subgroups, (ii) optionally, genetically modifying each subgroup of plant cells comprising regenerative plant cells, (iii) extracting DNA and/or RNA from a sample of said plant cells comprising regenerative plant cells of each subgroup, (iv) identifying one or more subgroups with the highest concentration of a desired nucleic acid sequence, and (v) selecting one or more subgroups of said cells with the highest concentration of the desired nucleic acid sequence,
whereby, optionally, and in another embodiment preferably, the plant cells comprising regenerative plant cells are plant cells comprising genetically modified regenerative cells.

In one embodiment, the plant cells are of monocotyledonous or dicotyledonous origin. In one embodiment, the regenerative plant cells are of monocotyledonous or dicotyledonous origin.

In one embodiment, the population of cells, e.g., monocotyledonous or dicotyledonous, cells are derived from one or more independent genetic modification events, for example, particle bombardments, e.g., particle gun shots, or protoplast transfection batches. Each modification event, e.g., each transformation or transfection event, can be used as subgroup. For example, the cells of one shot build one subgroup. In one embodiment, the population of regenerative plant cells are monocotyledonous or dicotyledonous and the regenerative plant cells are derived from one or more independent genetic modification events, for example, particle bombardments, e.g., particle gun shots, or protoplast transfection batches.

Accordingly, in one embodiment, the method of the invention comprises a step (a) comprising, preferably consisting of, the steps:
a) (i) providing a population of plant cells comprising regenerative plant cells, (ii) dividing the population of the plant cells comprising regenerative plant cells into subgroups and treating the cells of each subgroup in a genetically modifying method, e.g. particle bombardment, Agrobacterium transformation, or protoplast transfection, (iii) extracting DNA and/or RNA from a sample from said cells of each subgroup, (iv) identifying one or more subgroups with the highest concentration of the desired nucleic acid sequence, and (v) selecting one or more subgroups of said cells with the highest concentration of the desired nucleic acid sequence.

In another embodiment, a population of cells treated in a genetic modification method, e.g., resulting from a particle bombardment, or an Agrobacterium transformation, or a protoplast transfection, are divided into different subgroups. For example, the cells of one shot are divided into several subgroups or cells of several shots are mixed before they are divided into subgroups.

In one embodiment of the present invention, the concentration of the desired nucleic acid sequence is determined by taking a sample of the subgroup's cells, extracting the nucleic acid molecules of these cells and determining the concentration of the desired nucleic acid sequence in the extract. The extraction of the DNA or RNA from the cells results in material that cannot be regenerated, e.g., requires the destruction of the cells.

In one embodiment, the method of the present invention comprises picking a sample from said plant cells comprising regenerative plant cells, e.g., picking 0.1%, 0.5%, 1,0%, 5,0%, 10%, 15%, 20%, 30%, 40%, or 50%, or more, if appropriate. Preferably, the sample picked is as small as possible, but sufficiently large enough to determine in a quantifiable manner the concentration of the nucleic acid sequence. The size can for example be dependent on the efficiency of the genetic modification step, e.g., between 1% to 15% of the cells per subgroup. In one embodiment, the sample that is picked comprises cells comprising regenerative plant cells.

In one embodiment, the sample that is picked comprises only or mainly regenerative plant cells if it is desired to determine the concentration of the desired nucleic acid sequence in samples of cells that represent the cells that are particularly suitable for plant regeneration. In one embodiment, the picked sample comprises only or mainly cells that are non-regenerative plant cells if it desired to select a sample that avoids a reduction of those cells that are reproducible, for example, if the non-regenerative cells represent the concentration of the desired nucleic acid molecule in the non-regenerative cells.

In the method if the present invention, basically all cell types capable of division and proliferation can be used. In general, all regenerative cells can be used in the method of the present invention, no matter if the regeneration takes place via organogenesis (e.g. shoots) or through somatic/gametic embryogenesis. The present invention can be adapted accordingly. The regenerative cells used in the method of the invention are plant cells.

The regenerative plant cells are, for example, selected from the group consisting of
single cells e.g., protoplasts or microspores,
cell aggregates e.g., cell suspensions or callus cultures,
complex multicellular explants from mature or immature seeds e.g., immature embryos, scutella or cotyledons,
complex multicellular explants from seedlings e.g., roots, hypocotyls, cotyledons, leaves, petioles or meristems, and
complex multicellular explants from plants e.g., roots, leaves, leaf-bases, petioles, stems, or meristems.

Thus, preferable, the regenerative plant cells are selected form the group consisting of protoplasts, microspores, cell suspensions, callus cultures, immature embryos, scutella, cotyledons, roots, leaves, leaf-bases, petioles, stems, meristems, roots, leaves, leaf-bases, petioles, stems, and meristems.

The regenerative cells used in the method of the invention is not limited to these examples.

Accordingly, the method of the invention, further, comprises a step (b) culturing the regenerative cells of the subgroup selected in step (a) and generate, for example, calli, or regenerated shoots.

According to the present invention, in one embodiment, plant regeneration is initiated to produce plants. Plant regeneration can be started from genetically modified, e.g., transformed or transfected, regenerative cells. It can be started as well from non-modified, e.g., nontransformed, tissues to select cells with desirable traits already existing in the plants.

The “recovering” or “regeneration” as used herein means generating or generation of a plant by in vitro culturing regenerative plant cells, e.g. from protoplasts or callus tissue. It can for example be achieved via somatic embryogenesis or organogenesis. Methods for regenerating plants are well known in the art, e.g. in Haberlandt, G. (1902), Sitzungsber Akad. Wiss. Wien. Math. Nat. 111, 69-91; Skoog and Miller (1957), Symp. Soc. Exp. Biol. 54, 118-130; Steward et al. (1958), Am. J. Bot. 45, 705-708. Key components to develop a medium for in vitro plant regeneration are basal medium, carbon source, plant growth regulators, and other added supplements to improve the regeneration step. Explants enter the induction phase, during which cells are identified to produce for example callus, shoots, roots, or embryos. Explants or derived callus enter the realization stage, which results in the appearance of shoots, roots and embryos allowing to recover and grow plants. To improve the efficiency of plant cell regeneration after transformation or genome editing, expression of genes encoding proteins involved in plant growth regulation were proven to be useful, in particular so called morphogenic genes (Gordon Kamm et al, Plants 2019, 8, 38; doi: 10.3390/plants8020038). Transcription factors like WUSCHEL (WUS) and related WOX, BABY BOOM or LEC1 are known to promote cell development and somatic embryogenesis when overexpressed in monocotyledonous plants. This property has been used to enhance the efficiency of genome editing in plants.

Typically plant cells are transformed with a gene construct encoding a CRISPR-Cas complex and, optionally, with a gene construct encoding a morphogenic gene (WO 2018/224001, WO 2021/030242, WO 2021/022043). For example, ribonucleoproteins (RNP) are introduced or a nucleic acid encoding a functional CRISPR-Cas system can be expressed (Kanchiswami Plant Cell Rep 2016 Vol. 35 Issue 7 Pages 1469-74; Liang et al. Nat Commun 2017 8:14261). Accordingly, the present invention includes increasing the regeneration efficiency of a genome edited plant cell. For example, the method of the present invention comprises in the step of modifying the nucleic acid sequence of the regenerative cell introducing an endonuclease designed for a targeted genome modification and a transcription factors, e.g., WUSCHEL (WUS), WOX, BABY BOOM, LEC1, or others, in the form of a protein or its encoding mRNA, into a plant cell, further comprising the modification of the genome of said plant cell by the endonuclease, and regenerating said plant cell into a plant, thereby allowing the transcription factors to increase efficiency of regeneration. In a particular embodiment, the increased regeneration efficiency is an increased percentage of modified plant cells that regenerate into mature plants, compared to the percentage of control plants regenerated from genome edited plant cells that had undergone the same procedure except that they were not treated with a transcription factors, e.g., WUSCHEL (WUS), WOX, BABY BOOM, or LEC1.

In one embodiment, before the regenerative plant cells are cultured, e.g., before calli, or shoots are generated from the regenerative cells, the populations of cells of the selected subgroups of cells are divided into secondary subgroups. The concentration of the desired nucleic acid sequence is determined in said secondary subgroups as described before. Preferably, the concentration is determined by a molecular screen, e.g., ddPCR or NGS. One or more secondary subgroups with the highest concentration of the desired nucleic acid sequence is then selected and used to regenerate plants from the regenerative plant cells.

In one embodiment, after the regenerative plant cells are selected from the selected subgroup or subgroups of step (a), i.e. before calli, or shoots or plants are generated from the regenerative cells from the selected subgroup or subgroups of step (a), the regenerated cells are divided into subgroups and the presence of the nucleic acid sequence is determined as described before, preferably by a molecular screen, e.g., ddPCR or NGS. One or more subgroups of cells with the highest concentrations of the desired nucleic acid sequence is then selected to generate plants (step (c)).

A preferred method to determine the quantity of the desired nucleic acid sequence is ddPCR or NGS.

Thus, in one embodiment, the method of the invention comprises a step (b), taking samples from plant cells comprising the cultured cells, e.g. from calli or shoots regenerated from the cells of the selected subgroup of step (a), extracting the DNA of the cells of the sample, and determining the quantity of the desired nucleic acid molecule by ddPCR.

The concentration of the desired nucleic acid sequence in each sample of the subgroup comprising the regenerated cells taken in step (b) can be determined as described, e.g., according to the same principles as for the regenerative cells of step (a). For example, the concentration is determined after extracting the nucleic acid molecules, e.g., the DNA, from the cells of said subgroups. The concentration of the desired nucleic acid sequence is preferably determined in a PCR, in particular a digital PCR, preferably a ddPCR, or by sequencing the nucleic acid sequence, e.g., via NGS.

Accordingly, in one embodiment, the method of the invention comprises a step (b) comprising, preferably consisting of, the steps:

i) culturing the regenerative cells of the one or more selected subgroup(s) in step (a), (ii) extracting nucleic acid molecules, e.g., DNA, from a sample of said cultured cells, e.g. calli or regenerated shoots, of each subgroup, (iii) identifying one or more subgroups with the highest concentration of the desired nucleic acid sequence, and (iv) selecting one or more subgroups of said cultured cells with the highest concentrations of the desired nucleic acid sequence.

Accordingly, in one embodiment, the method of the invention comprises a step (a) and a step (b), comprising the steps:
a) (a1) (i) providing a population of plant cells comprising regenerative plant cells, (ii) dividing the population of the plant cells comprising regenerative plant cells into subgroups, (iii) extracting nucleic acid molecules, e.g., DNA, from cells from one or more samples from said plant cells comprising regenerative cells of each subgroup, (iv) identifying one or more subgroups with the highest concentration of the desired nucleic acid sequence, and (v) selecting one or more subgroups of said plant cells comprising regenerative plant cells with the highest concentration of the desired nucleic acid sequence,
   whereby, optionally, the plant cells and/or the regenerative plant cells are genetically modified plant cells, or
   (a2) (i) Dividing the population plant cells comprising regenerative plant cells into subgroups, (ii) optionally, genetically modifying the cells of the subgroups of plant cells comprising regenerative plant cells, (iii) extracting nucleic acid molecules, e.g., DNA, from one or more samples from said cells of each subgroup, (iv) identifying one or more sub-groups with the highest concentration of a desired nucleic acid sequence, and (v) selecting one or more subgroups of said cells with the highest concentration of the desired nucleic acid sequence, and
b) (i) culturing cells, e.g. recovering calli or shoots from the regenerative cells, from the selected subgroup(s) from step (a), (ii) extracting nucleic acid molecules, e.g. DNA, from cells of one or more samples from each subgroup, (iii) identifying one or more subgroups with the highest concentrations of the desired nucleic acid sequence, and (iv) selecting one or more subgroups of said cells with the highest concentration of the desired nucleic acid sequence,
whereby, optionally, the regenerative plant cells are genetically modified regenerative plant cells, e.g., cells resulting from a particle bombardment or an Agrobacterium transfection, for example, resulting from a gene editing process.

Further, the method of the present invention allows recovering plants from the subgroup(s) selected in step (b). Accordingly, in one embodiment, the method of the present invention comprises a step (c) comprising, preferably consisting of culturing, recovering and/or growing individual plants from one or more selected subgroups of regenerated plant cells, e.g., regenerated calli or shoots.

According to the present invention, nucleic acid molecules, e.g. DNA, are extracted from one or more samples taken from a subgroup. The extracted nucleic acid molecules, e.g., the extracted DNA, from one or more samples derived from one subgroup can be pooled. Also, all cells of samples that are taken from one subgroup can be pooled and the nucleic acid molecules, e.g., the DNA, is then extracted from the pooled cells.

Thus, in one embodiment, one or more subgroups with the highest concentration(s) of the desired nucleic acid sequence are identified and the cells, e.g. regenerated calli or shoots, of the subgroup(s) are used to regenerate plants.

According to the present invention, in one step, after the plants have been recovered in step (c), the presence of the desired nucleic acid sequence in individual plants is determined from the nucleic acid molecules, e.g., DNA, that are extracted from one or more samples from said plants. The presence can be determined according well-known methods. The presence of the desired nucleic acid sequence is determined preferably in a molecular screen, e.g., in a PCR, preferably ddPCR, or by sequencing the nucleic acid sequence, e.g., via NGR.

In one embodiment, the method of the present invention comprises one further step (ba) between steps (b) and (c):
(i) recovering individual plants from the cells, e.g. the calli, or shoots, of the selected subgroup(s) from step (b), (ii) dividing the population of plants into subgroups, (ii) extracting DNA from one or more samples from said plants of each subgroup, (iii) pool the samples taken from individual plants from one subgroup and identifying one or more subgroups with the highest concentration(s) of the desired nucleic acid sequence, and (iv) selecting one or more subgroups of said plants with the highest concentration of the desired nucleic acid sequence.

In one embodiment, in a following step, (v), individual plants are selected from the subgroup with the highest concentration of the desired nucleic acid sequence.

Accordingly, in one embodiment, according to the method of the present invention, step (c) as described follows step (ba).

For the determination of the presence and, if desired, the concentration of the desired nucleic acid sequence in a plant sample, the plant tissue is harvested, and the nucleic acid molecules are extracted from these samples. Preferably, the samples are taken in a manner that allows a quantitative analysis.

Accordingly, in one embodiment, the method of the present invention preferably comprises the steps:
(a1) (i) providing a population of plant cells comprising regenerative plant cells expected to have a desired nucleic acid sequence, (ii) dividing the population of the plant cells comprising regenerative plant cells into subgroups, (iii) extracting DNA from one or more samples from said cells of subgroups, (iv) identifying one or more subgroups with the highest concentration(s) of the desired nucleic acid sequence, and (v) selecting one or more subgroups of said cells with the highest concentration of the desired nucleic acid sequence,
whereby, optionally, cells are genetically modified cells, preferably genetically modified regenerative plant cells,
or
(a2) (i) Dividing the population plant cells comprising regenerative plant cells into subgroups, (ii) optionally, genetically modifying the subgroups of plant cells comprising regenerative plant cells, (iii) extracting DNA from one or more samples from said cells of each subgroup, (iv) identifying one or more subgroups with the highest concentration(s) of a desired nucleic acid sequence, and (v) selecting one or more subgroups of said cells with the highest concentration(s) of the desired nucleic acid sequence,
and
(b) (i) culturing the regenerative plant cells of the selected subgroup(s) of step (a1) or (a2), (ii) extracting DNA from one or more samples from the cultured plant cells, e.g. regenerated calli or shoots, of said subgroups, (iv) identifying one or more subgroups with the highest concentration(s) of the desired nucleic acid sequence, and (v) selecting one or more subgroups of said cells, e.g. regenerated calli, or shoots, with the highest concentration(s) of the desired nucleic acid sequence,
and,
(c1) (i) recovering individual plants or shoots from the regenerated cells, e.g., regenerated calli or shoots, of the selected subgroup(s) from step (b), (ii) dividing the population of plants or shoots into subgroups, (ii) extracting DNA and/or RNA from one or more samples from cells of each subgroup, (iii) identifying one or more subgroups with the highest concentration(s) of the desired nucleic acid sequence, and (iv) selecting one or more subgroups of said plants or shoots with the highest concentration(s) of the desired nucleic acid sequence and grow plants thereof,
or
(c2), (i) recovering individual plants or shoots from the regenerated cells, e.g., calli, or regenerated shoots, of the selected subgroup(s) from step (b), (ii) taking DNA and/or RNA comprising samples from each plant or shoot, (iii) analyzing said DNA and/or RNA for presence of the desired nucleic acid sequence, (iv) selecting the plants with the desired nucleic acid sequence and grow plants thereof,
whereby, optionally, the plant cells are genetically modified cells, preferably regenerative plant cells, e.g., cells resulting from a particle bombardment or an Agrobacterium transformation or a protoplast transfection.

In one embodiment, step (b) is omitted. For example, step (b) is optional, if, the efficiency of the modification of the regenerative cells in step (a) as described herein is high. Step (b) can be omitted, for example, after an efficient bombardment.

As discussed, in one embodiment, the method also includes a step (ba), e.g. a step (a), e.g. (a1) or (a2), a step (ba) and a step (c), e.g. (c1) or (c2).

The culturing on step (c) of the present invention can, for example, take place in the greenhouse or in the field.

Accordingly, in one embodiment of the present invention, the regenerative cells are further development to individual clonal plants.

Accordingly, in one embodiment, the method of the invention comprises a step (a) as described herein, e.g., (a1) or (a2), and a step (c), e.g., (c1) or (c2), and, optionally, a step (b1) or (b2), e.g. a step (b1) and (ba) or a step (b2) and (ba), each in combination with a step (a1) or (a2). Thus, in one embodiment, the method of the invention consists of a step (a), e.g., (a1) or (a2), a step (b), and a step (c), e.g., (c1) or (c2), and, optionally, step (ba).

Thus, in one embodiment, the method of the invention comprises steps:
a1 and c1;
a1 and c2;
a2 and c1; or
a2 and c2,

In a preferred embodiment, the method of the invention comprises the steps
a1 and c1;
a1 and c2;
a2 and c1; or
a2 and c2,
in combination with step
b1 or b2.

For example, the method of the present invention comprises, preferably consists of the following steps:
a1, b1, c1;
a2, b1, c1;
a1, b2, c1;
a1, b1, c2;
a1, b2, c2,
a2, b1, c2;
a2, b2, c1; or
a2, b2, c2;
or any other combination of the steps in the order (a) to (c). In one embodiment, each of the combinations comprises a step (ba).

In one embodiment, the selected events are characterized molecularly, e.g., in a sequence specific analysis. These follow-up analyses will reveal for example allele-specific mutation profiles, and the type of modification on different sub-genomes.

According to the invention, “one or more samples” means, that the person skilled in the art chooses a number of samples as appropriate, considering the number of individual members of the group, and/or the efficiency of the modification step.

Accordingly, in one embodiment of the present invention, the method for the modification of the regenerative cell comprises a protein or DNA delivery as for example, by biolistic delivery. In one embodiment, the genome is edited, but no nucleic acid molecule is introduced into the genome of the target cell. Accordingly, in one embodiment, the present invention comprises a step of genetically modifying the cell using the CRISPR/Cas system, for example in base editing or prime editing.

In one embodiment, the regenerative cells used in the method of the invention are plant cells or cells derived from a plant, in particular monocotyledonous and dicotyledonous plants including fodder or forage legumes, ornamental plants, food crops, trees or shrubs. According to an embodiment of the present invention, the plant is a crop plant.

Accordingly, in one embodiment, the regenerative cells are monocotyledonous regenerative cells. According to another embodiment of the present invention, the plant produced is a cereal or derived thereof. Preferred are monocotyledonous crop plants. According to another embodiment of the present invention, the plant is sugarcane, cereals include rice, maize, wheat, barley, millet, rye, triticale, sorghum, emmer, spelt, einkorn, teff, milo and oats. In a particular embodiment the plant or plants of the invention, or used in the methods of the invention, are selected from the group consisting of maize, wheat, rice, soybean, cotton, oilseed rape including canola, sugarcane, sugar beet and alfalfa.

RNP-mediated genome editing, and plant regeneration is shown successfully for several monocots, e.g., rice (Banakar, et al., 2020, Rice 13:4.), maize (Svitashev et al., 2016, Nat. Commun. 7:13274) and wheat (Liang et al., 2017, Nat. Commun. 8:14261) using particle bombardment.

For example, the monocotyledonous regenerative cells are for example, selected from the group consisting of
single cells e.g., protoplasts or microspores,
cell aggregates e.g., cell suspensions or callus cultures,
complex multicellular explants from mature or immature seeds e.g., immature embryos, scutella or cotyledons,
complex multicellular explants from seedlings e.g., roots, hypocotyls, cotyledons, leaves, petioles or meristems, and
complex multicellular explants from plants e.g., roots, leaves, leaf-bases, petioles, stems, or meristems.

Thus, preferable, the monocotyledonous regenerative plant cells are selected form the group consisting of protoplasts, microspores, cell suspensions, callus cultures, immature embryos, scutella, cotyledons, roots, leaves, leaf-bases, petioles, stems, meristems, roots, leaves, leaf-bases, petioles, stems, and meristems.

In one embodiment, the monocotyledonous regenerative cells are monocotyledonous regenerative cells that are genetically modified, e.g., transformed and/or transfected monocotyledonous regenerative cells. In one embodiment, mutated monocotyledonous regenerative cells are cells that are cells bombarded in a biolistic delivery system.

In one preferred embodiment, the cells used in the method of the present invention are wheat cells or derived from wheat cells.

In another embodiment, the regenerative cells are dicotyledonous regenerative cells.

Efficient methods for the production of genome-edited dicotyledonous plants are well known to those skilled in the art, using CRISPR DNA components delivered with Agrobacterium as, e.g. for oilseed rape (Braatz et al. 2017, Plant Physiol. Jun; 174 (2): 935-942), cotton (Li et al., 2017, Sci Rep. March 3; 7:43902.), or biolistics, as e.g. for soybean (Li et al., 2015, Plant Physiol. Oct; 169 (2): 960-970), or protoplast transfection, as e.g. for potato (Anderson et al., Plant Cell Reports volume 36, 117-128), or as CRISPR ribonucleoproteins for DNA-free genome editing in protoplasts (RNP), as e.g. for lettuce (Woo et al., 2015, Nat. Biotechnol. 33:1162-1164), cabbage (Park et al. 2019, Plant Biotechnol. Rep. 13:483-489) or potato (Gonzalez, et al., 2020, Front. Plant Sci. 10:1649).

Preferred are dicotyledonous crop plants. Examples of crop plants include but are not limited to chicory, carrot, cassava, trefoil, soybean, beet, sugar beet, sunflower, canola, alfalfa, rapeseed, linseed, cotton, tomato, potato, tobacco, and Brassica species, e.g. Brassica oleracea or Brassica napus or Brassica juncea.

Accordingly, the method of the invention, thus, comprises recovering cultured callus or shoots from said regenerative dicotyledonous cells in one or more sub-group with the highest concentration(s) of the desired nucleic acid sequence, and selecting one or more sub-groups with the highest concentration(s) of presence of the desired nucleic acid sequence. Thus, the plant tissue that is cultured in step (b) of the present invention is in one embodiment, calli or shoots.

In one embodiment, the dicotyledonous regenerative cells are dicotyledonous regenerative cells that are mutated, e.g., transformed or transfected, dicotyledonous regenerative cells. The mutated dicotyledonous regenerative cells can be produced for example in a biolistic delivery system, or produced in transfection of protoplasts, microspores etc., or other methods known in the art or described herein below. Accordingly, in one embodiment, mutated dicotyledonous regenerative cells are cells that are bombarded in a biolistic delivery system.

Accordingly, the present invention also relates to a method for the production of dicotyledonous shoots with a desired nucleic acid sequence and growing dicotyledonous plants from the selected shoots.

Regenerative dicotyledonous cells used in the method of the present invention could be single cells e.g., protoplasts, microspores, cell aggregates (cell suspensions and callus cultures), multicellular seedling explants (roots, hypocotyls, cotyledons, leaves and meristems), multicellular plant explants (leaves, stems, petioles), or mature seed explants (meristems, cotyledons).

Accordingly, in one embodiment, the method of the invention, thus, comprises the following steps:

a)
Introducing into a population of plant cells comprising regenerative plant cells, e.g., explants, a composition comprising a system capable of introducing a mutation at a predefined site in the genome,
Picking a sample form said cells, e.g., picking 0.1%, 0.5%, 1,0%, 5,0%, 10%, 15%, 20%, 30%, 405, 50%, 60%, 70% and up to 80% (preferably as little as possible dependent on the efficiency of the genetic modification step, e.g., between 1% to 15%) of the cells per plate,
Extracting DNA from said sample cells, wherein, optionally, the respective cell is completely used for extraction, and pooling the DNA of cells from one or more plates, or pooling the cells for DNA extraction wherein, optionally, a respective cell taken is completely used for extraction, preferably thereby extracting the DNA from said pool of cells, such that a single dish or a single shot performed with a dish of cells can be identified as source of the samples, and
Analyzing said extracted DNA in a molecular screen, e.g. using ddPCR or NGS, for the concentration of a desired sequence, and determine the concentration of the mutation or sequences the predefined position in the genome,
Selecting plates, for example, take best 1%, 5%, 10%, or 25%, for further culturing based on result of said analysis, and

b)
growing calli, shoots, roots and/or plants in tissue culture from the regenerative cells of the selected plates, and
Picking a sample form said regenerated callus, shoots, roots and/or plants in tissue culture, e.g., picking 0.1%, 0.5%, 1,0%, 5,0%, 10%, 15%, 20%, 30%, 405, 50%, 60%, 70% and up to 80% (preferably as little as possible dependent on the efficiency of the genetical modification step, e.g., between 1% to 15%) of the plant material, e.g., regenerated callus, shoots, roots and/or plants in tissue culture, per box,
Extracting DNA from the cells in said sample of regenerated callus, shoots, roots and/or plants in tissue culture, wherein a cell in the sample of regenerated callus, shoots, roots and/or plants in tissue culture is completely used for extraction, and pooling the DNA of the cells from one or more boxes, or pooling the cells for DNA extraction, preferably thereby extracting the DNA from said pool of cells such that a single dish or a single shot performed with a dish of cells can be identified as source of the samples, and
Analyzing said extracted DNA using ddPCR or NGS for the presence of a desired mutation or sequence at the predefined position in the genome, and determine the concentration of the mutation or sequences the predefined position in the genome,
Selecting a box with best or highest concentration of the mutation or desired sequence, for example, take best 1%, 5%, 10%, or 25%, for further culturing based on result of said analysis,
and

c)
Further culturing and regenerating the regenerated callus, shoots, roots and/or plants in tissue culture into plant, e.g., further development of individual clonal plants,
Picking samples from the plants, e.g., from the leaves, preferably, pick leave samples of the same size, e.g., leave disks,
Extracting the DNA from the plant samples,
Analyzing the DNA and determine the concentration of the desired mutation or sequence at the predefined position, and
Selecting a plant that comprises the mutation or desired sequence,
wherein step (b) is optionally. In a preferred embodiment, the method of the present invention comprises the steps (a), (b) and (c).

In one embodiment of the present invention comprises that in step (c) a plant is selected that also shows preferred phenotypic or agronomic features, e.g., increased yield, stress tolerance etc. Accordingly, step (c) can, for example, be used for determining the individual genotype (clonal characterization) of a specific sample, e.g., plant sample. Accordingly, in one embodiment, the samples taken in step (c) are not pooled and each sample is analysed individually, e.g., if the plants are growing in the greenhouse

Accordingly, according to the method of the invention step (a) and (b) are used to reduce the number of plants to be screened in step (c), hence lowering the cost and increasing the through-put.

Further, in one embodiment, in the method of the present invention that comprises the steps (a) to (c) as described herein, the product of step (a) is directly used in step (b) without any additional processing of the product of step (a) before step (b).

In another embodiment, the product of step (b) of the method of the present invention is directly used in step (c) of the method of the present invention without any additional processing of the product before it is used in step (c). In one embodiment, in the method of the present invention, the products of step (a) are used directly in step (b) and the products of step (b) are directly used in the method's step (c). Accordingly, in one embodiment, there is no modification of the products of step (a) and step (b). Accordingly, the method of the present invention in one embodiment consists of steps (a) to (c).

Examples of plants of which the cells can be used in the methods of the present invention include Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g., Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g., Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g., Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g., Glycine max, Soja hispida or Soja max), Gossypium hirsutum, Helianthus spp. (e.g., Helianthus annuus), Hemerocallis fulva, Hibiscus spp., Hordeum spp. (e.g., Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g., Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g., Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g., Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g., Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

The present invention also provides plants produced by the methods of the invention. Such plants can have improved phenotypic or agronomic traits, e.g., as result of the genetic modification of the regenerative cells in step (a) of the method of the present invention. A trait of particular economic interest is increased yield. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance and early vigour may also be important factors in determining yield. Optimizing the abovementioned factors may therefore contribute to increasing crop yield. Another important trait for many crops is early vigour. Improving early vigour is an important objective of modern rice breeding programs in both temperate and tropical rice cultivars. Long roots are important for proper soil anchorage in water-seeded rice. Where rice is sown directly into flooded fields, and where plants must emerge rapidly through water, longer shoots are associated with vigour. Where drill-seeding is practiced, longer mesocotyls and coleoptiles are important for good seedling emergence. The ability to engineer early vigour into plants would be of great importance in agriculture. A further important trait is that of improved abiotic stress tolerance. Abiotic stress is a primary cause of crop loss worldwide, reducing average yields for most major crop plants by more than 50% (Wang et al., Planta 218, 1-14, 2003). Abiotic stresses may be caused by drought, salinity, extremes of temperature, chemical toxicity and oxidative stress. The ability to improve plant tolerance to abiotic stress would be of great economic advantage to farmers worldwide and would allow for the cultivation of crops during adverse conditions and in territories where cultivation of crops may not otherwise be possible. Another trait of importance is resistance to biotic stress, typically caused by pathogens, such as bacteria, viruses, fungi, plants, nematodes and insects, or other animals, which may result in negative effects on plant growth.

Plants produced by the methods of the invention may also have improved nutritional traits such as higher content in protein, minerals, vitamins, micronutrients, essential amino acids levels or other health-promoting compounds.

The present invention extends further to encompass the progeny of the cell, tissue, organ or whole plant that has been produced by any of the aforementioned methods, the only requirement being that the progeny exhibit the same characteristics as the parent plant (i.e. at least the same genome modification and/or the same improved agronomic traits), provided however that the plant is produced by the methods of the invention and not by essentially biological processes, and provided that its progeny is different from, and can be discriminated from naturally occurring plants.

The invention also extends to harvestable parts of a plant produced by any of the aforementioned methods, such as, but not limited to seeds, leaves, fruits, flowers, stems, roots, rhizomes, tubers and bulbs, which harvestable parts comprise the desired edit or modification in the genome.

The invention furthermore relates to products derived or produced, preferably directly derived or directly produced, from a harvestable part of such a plant, such as dry pellets, pressed stems, meal or powders, oil, fat and fatty acids, carbohydrates, sap, juice or proteins. Preferred carbohydrates are starch, cellulose or sugars, preferably sucrose. Also preferred products are residual dry fibres, e.g., of the stem (like bagasse from sugar cane after cane juice removal), molasse, or filter cake for example from sugar cane. In one embodiment the product still comprises the genome or part thereof that comprises the desired edit or modification, which is useful for example as an indicator of the particular quality of the product. In another embodiment, the product derived or produced from a plant according to the invention is different from the product derived or produced from a plant not comprising the genome edit.

The invention also includes methods for manufacturing a product comprising a) growing the plants of the invention and b) producing said product from or by the plants of the invention or parts thereof, such as stem, root, leaves and/or seeds. In a further embodiment the methods comprise the steps of a) growing the plants of the invention, b) removing the harvestable parts as described herein from the plants and c) producing said product from, or with the harvestable parts of plants according to the invention.

In a further embodiment the products produced by the manufacturing methods of the invention are plant products such as, but not limited to, a foodstuff, feedstuff, a food supplement, feed supplement, fibre, cosmetic or pharmaceutical. In another embodiment the methods for production are used to make agricultural products such as, but not limited to, plant extracts, proteins, amino acids, carbohydrates, fats, oils, polymers, vitamins, and the like.

In yet another embodiment the nucleic acid comprising the desired edit or modification produced using the methods of the invention is comprised in an agricultural product. In a particular embodiment the nucleic acid comprising the desired edit or modification of the invention may be used as product marker, for example where an agricultural product was produced by the methods of the invention. Such a marker can be used to identify a product to have been produced by an advantageous process resulting not only in a greater efficiency of the process but also improved quality of the product due to increased quality of the plant material and harvestable parts used in the process. Such markers can be detected by a variety of methods known in the art, for example but not limited to PCR based methods for nucleic acid detection or antibody-based methods for protein detection.

The present invention also encompasses use of the nucleic acid comprising the desired edit or modification, it may find use in breeding programmes in which a DNA marker is identified which may be genetically linked to the nucleic acid or locus on the genome comprising the desired edit or modification. The nucleic acid comprising the desired edit or modification itself may be used to define a molecular marker. This DNA marker may then be used in breeding programmes to select plants having one or more enhanced yield-related traits as defined herein in the methods of the invention. Nucleic acids acid comprising the desired edit or modification may also be used as probes for genetically and physically mapping the genes that they are a part of, and as markers for traits linked to those genes. Such information may be useful in plant breeding in order to develop lines with desired phenotypes.

The present invention also provides a platform for producing genome edited plants, comprising a module for selecting the target gene for editing and designing a suitable gRNA that allows the desired modification of the target gene, a second module for performing the gene edit comprising preparation of the cells to be edited, introduction of the endonuclease designed for a desired genome modification, e.g. together with a TaWOX, in the form of protein or mRNA according to the methods of the present invention, a third module for regenerating the edited cells into plants and a screening system for selecting the plants having the desired genome edit. The genome edited plants produced with this platform can subsequently used in breeding programs.

The present invention also encompasses that seeds form the plants produced by the present invention are sown and progeny populations are grown. For example, the seeds are grown in a greenhouse or in the field. Samples, e.g. from leaves or seeds, from the produced progenies can be analysed, e.g. by NGS. Preferably, plants are selected from the progenies that comprise the predicted mutations and that segregated as expected.

### DEFINITIONS AND FURTHER EMBODIMENTS

Methods of introducing for a desired genome or genetic modification, either as protein or as mRNA, can be achieved by chemical means, non-chemical means or by physical means. Chemical means for introducing proteins or nucleic acids in a cell usually rely on uptake by endocytosis or on merger with the cell membrane. Non limiting examples of chemical means for introduction include lipofection, polyethyleneimine (PEI)-mediated introduction, polyethylene glycol (PEG)-mediated introduction, nucleofection, calcium phosphate precipitation, liposomes, immunoliposomes, fusion, polycation or lipid: nucleic acid conjugates, cell-penetrating peptides, and DEAE-dextran mediated transfection. Non-chemical means encompass methods that create pores in the cell membrane or spots of increased membrane permeability. Well known examples are electroporation, sonoporation (by cavitation of gas bubbles), and use of laser light. Non limiting examples of physical means for introduction include microinjection, nanoparticlemediated delivery, particle gun technology (biolistics) and impalefection (trough use of needle-like nanostructures coated with the compound(s) of interest. For example, a desired genome modification can be introduced by physical means, preferably by biolistics. Biolistics or particle bombardment is a tool for delivering compound(s) into a cell by coating the compound(s) on metal microparticles (usually tungsten or gold particles) and shooting these coated particles with a gene gun into the cell. This technique has been proven to be very useful for transforming plants that are otherwise hard to transform or regenerate, as well as for transformation of cell organelles such as protoplasts (for a review, see Ozyigit and Kurtoglu, Mol Biol Rep. 47 (12): 9831-9847, 2020). Another useful method is aerosol beam microinjection (U.S. Pat. No. 5,240,842).

Abbreviations: GFP-green fluorescence protein, GUS-beta-Glucuronidase, BAP-6-benzylaminopurine; 2,4-D-2,4-dichlorophenoxyacetic acid; MS-Murashige and Skoog medium; NAA-1-naphtaleneacetic acid; MES, 2-(N-morpholino-ethanesulfonic acid, IAA indole acetic acid; Kan: Kanamycin sulfate; GA3-Gibberellic acid; Timentin™: ticarcillin disodium/clavulanate potassium, microl: Microliter.

It is to be understood that this invention is not limited to the particular methodology or protocols. It is also to be understood that the terminology used herein is for the purpose of describing particular embodiments only and is not intended to limit the scope of the present invention which will be limited only by the appended claims. It must be noted that as used herein and in the appended claims, the singular forms “a,” “and”, and “the” include plural reference unless the context clearly dictates otherwise. Thus, for example, reference to “a vector” is a reference to one or more vectors and includes equivalents thereof known to those skilled in the art, and so forth. The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. For clarity, certain terms used in the specification are defined and used as follows:

Donor DNA molecule: As used herein the terms “donor DNA molecule”, “repair DNA molecule” or “template DNA molecule” all used interchangeably herein mean a DNA molecule having a sequence that is to be introduced into the genome of a cell. It may be flanked at the 5′ and/or 3′ end by sequences homologous or identical to sequences in the target region of the genome of said cell. It may comprise sequences not naturally occurring in the respective cell such as ORFs, non-coding RNAs or regulatory elements that shall be introduced into the target region or it may comprise sequences that are homologous to the target region except for at least one mutation, a gene edit: The sequence of the donor DNA molecule may be added to the genome or it may replace a sequence in the genome of the length of the donor DNA sequence.

Double-stranded RNA: A “double-stranded RNA” molecule or “dsRNA” molecule comprises a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule.

Endogenous: An “endogenous” nucleotide sequence refers to a nucleotide sequence, which is present in the genome of the untransformed plant cell.

Enhanced expression: “enhance” or “increase” the expression of a nucleic acid molecule in a plant cell are used equivalently herein and mean that the level of expression of the nucleic acid molecule in a plant, part of a plant or plant cell after applying a method of the present invention is higher than its expression in the plant, part of the plant or plant cell before applying the method, or compared to a reference plant lacking a recombinant nucleic acid molecule of the invention. For example, the reference plant is comprising the same construct which is only lacking the respective NEENA. The term “enhanced” or “increased” as used herein are synonymous and means herein higher, preferably significantly higher expression of the nucleic acid molecule to be expressed. As used herein, an “enhancement” or “increase” of the level of an agent such as a protein, mRNA or RNA means that the level is increased relative to a substantially identical plant, part of a plant or plant cell grown under substantially identical conditions, lacking a recombinant nucleic acid molecule of the invention, for example lacking the NEENA molecule, the recombinant construct or recombinant vector of the invention. As used herein, “enhancement” or “increase” of the level of an agent, such as for example a preRNA, mRNA, rRNA, tRNA, snoRNA, snRNA expressed by the target gene and/or of the protein product encoded by it, means that the level is increased 50% or more, for example 100% or more, preferably 200% or more, more preferably 5 fold or more, even more preferably 10 fold or more, most preferably 20 fold or more for example 50 fold relative to a cell or organism lacking a recombinant nucleic acid molecule of the invention. The enhancement or increase can be determined by methods with which the skilled worker is familiar. Thus, the enhancement or increase of the nucleic acid or protein quantity can be determined for example by an immunological detection of the protein. Moreover, techniques such as protein assay, fluorescence, Northern hybridization, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) can be employed to measure a specific protein or RNA in a plant or plant cell. Depending on the type of the induced protein product, its activity or the effect on the phenotype of the organism or the cell may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry O H et al. (1951) J Biol Chem 193:265-275) or measuring the absorption of CBB G-250 (Bradford M M (1976) Analyt Biochem 72:248-254). As one example for quantifying the activity of a protein, the detection of luciferase activity is described in the Examples below.

Expression: “Expression” refers to the biosynthesis of a gene product, preferably to the transcription and/or translation of a nucleotide sequence, for example an endogenous gene or a heterologous gene, in a cell. For example, in the case of a structural gene, expression involves transcription of the structural gene into mRNA and—optionally—the subsequent translation of mRNA into one or more polypeptides. In other cases, expression may refer only to the transcription of the DNA harbouring an RNA molecule.

Expression construct: “Expression construct” as used herein mean a DNA sequence capable of directing expression of a particular nucleotide sequence in an appropriate part of a plant or plant cell, comprising a promoter functional in said part of a plant or plant cell into which it will be introduced, operatively linked to the nucleotide sequence of interest which is-optionally-operatively linked to termination signals. If translation is required, it also typically comprises sequences required for proper translation of the nucleotide sequence. The coding region may code for a protein of interest but may also code for a functional RNA of interest, for example RNAa, siRNA, snoRNA, snRNA, microRNA, ta-siRNA or any other noncoding regulatory RNA, in the sense or antisense direction. The expression construct comprising the nucleotide sequence of interest may be chimeric, meaning that one or more of its components is heterologous with respect to one or more of its other components. The expression construct may also be one, which is naturally occurring but has been obtained in a recombinant form useful for heterologous expression. Typically, however, the expression construct is heterologous with respect to the host, i.e., the particular DNA sequence of the expression construct does not occur naturally in the host cell and must have been introduced into the host cell or an ancestor of the host cell by a transformation event. The expression of the nucleotide sequence in the expression construct may be under the control of a constitutive promoter or of an inducible promoter, which initiates transcription only when the host cell is exposed to some particular external stimulus. In the case of a plant, the promoter can also be specific to a particular tissue or organ or stage of development.

Foreign: The term “foreign” refers to any nucleic acid molecule (e.g., gene sequence) which is introduced into the genome of a cell by experimental manipulations and may include sequences found in that cell so long as the introduced sequence contains some modification (e.g., a point mutation, the presence of a selectable marker gene, etc.) and is therefore distinct relative to the naturally occurring sequence.

Functional linkage: The term “functional linkage” or “functionally linked” is to be understood as meaning, for example, the sequential arrangement of a regulatory element (e.g., a promoter) with a nucleic acid sequence to be expressed and, if appropriate, further regulatory elements (such as e.g., a terminator or a NEENA) in such a way that each of the regulatory elements can fulfill its intended function to allow, modify, facilitate or otherwise influence expression of said nucleic acid sequence. As a synonym the wording “operable linkage” or “operably linked” may be used. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs. In a preferred embodiment, the nucleic acid sequence to be transcribed is located behind the promoter in such a way that the transcription start is identical with the desired beginning of the chimeric RNA of the invention. Functional linkage, and an expression construct, can be generated by means of customary recombination and cloning techniques as described (e.g., in Maniatis T, Fritsch E F and Sambrook J (1989) Molecular Cloning: A Laboratory Manual, 2nd Ed., Cold Spring Harbor Laboratory, Cold Spring Harbor (NY); Silhavy et al. (1984) Experiments with Gene Fusions, Cold Spring Harbor Laboratory, Cold Spring Harbor (NY); Ausubel et al. (1987) Current Protocols in Molecular Biology, Greene Publishing Assoc. and Wiley Interscience; Gelvin et al. (Eds) (1990) Plant Molecular Biology Manual; Kluwer Academic Publisher, Dordrecht, The Netherlands). However, further sequences, which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of a regulatory region for example a promoter and nucleic acid sequence to be expressed, can exist in a vector-integrated form and be inserted into a plant genome, for example by transformation.

Gene: The term “gene” refers to a region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

“Gene edit” when used herein means the introduction of a specific mutation at a specific position of the genome of a cell. The gene edit may be introduced by precise editing applying more advanced technologies e.g., using a CRISPR Cas system and a donor DNA, or a CRISPR Cas system linked to mutagenic activity such as a deaminase (WO15133554, WO17070632).

Genome and genomic DNA: The terms “genome” or “genomic DNA” is referring to the heritable genetic information of a host organism. Said genomic DNA comprises the DNA of the nucleus (also referred to as chromosomal DNA) but also the DNA of the plastids (e.g., chloroplasts) and other cellular organelles (e.g., mitochondria). Preferably the terms genome or genomic DNA is referring to the chromosomal DNA of the nucleus.

Heterologous: The term “heterologous” with respect to a nucleic acid molecule or DNA refers to a nucleic acid molecule which is operably linked to, or is manipulated to become operably linked to, a second nucleic acid molecule, e.g., a promoter to which it is not operably linked in nature, e.g., in the genome of a WT plant, or to which it is operably linked at a different location or position in nature, e.g., in the genome of a WT plant.

Preferably the term “heterologous” with respect to a nucleic acid molecule or DNA, e.g., a NEENA refers to a nucleic acid molecule which is operably linked to, or is manipulated to become operably linked to, a second nucleic acid molecule, e.g., a promoter to which it is not operably linked in nature.

A heterologous expression construct comprising a nucleic acid molecule and one or more regulatory nucleic acid molecule (such as a promoter or a transcription termination signal) linked thereto for example is a constructs originating by experimental manipulations in which either a) said nucleic acid molecule, or b) said regulatory nucleic acid molecule or c) both (i.e. (a) and (b)) is not located in its natural (native) genetic environment or has been modified by experimental manipulations, an example of a modification being a substitution, addition, deletion, inversion or insertion of one or more nucleotide residues. Natural genetic environment refers to the natural chromosomal locus in the organism of origin, or to the presence in a genomic library. In the case of a genomic library, the natural genetic environment of the sequence of the nucleic acid molecule is preferably retained, at least in part. The environment flanks the nucleic acid sequence at least at one side and has a sequence of at least 50 bp, preferably at least 500 bp, especially preferably at least 1,000 bp, very especially preferably at least 5,000 bp, in length. A naturally occurring expression construct—for example the naturally occurring combination of a promoter with the corresponding gene-becomes a transgenic expression construct when it is modified by non-natural, synthetic “artificial” methods such as, for example, mutagenization. Such methods have been described (U.S. Pat. No. 5,565,350; WO 00/15815). For example, a protein encoding nucleic acid molecule operably linked to a promoter, which is not the native promoter of this molecule, is considered to be heterologous with respect to the promoter. Preferably, heterologous DNA is not endogenous to or not naturally associated with the cell into which it is introduced but has been obtained from another cell or has been synthesized. Heterologous DNA also includes an endogenous DNA sequence, which contains some modification, non-naturally occurring, multiple copies of an endogenous DNA sequence, or a DNA sequence which is not naturally associated with another DNA sequence physically linked thereto. Generally, although not necessarily, heterologous DNA encodes RNA or proteins that are not normally produced by the cell into which it is expressed.

Hybridization: The term “hybridization” as defined herein is a process wherein substantially complementary nucleotide sequences anneal to each other. The hybridisation process can occur entirely in solution, i.e., both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g., photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tm) for the specific sequence at a defined ionic strength and pH. Medium stringency conditions are when the temperature is 20° C. below Tm, and high stringency conditions are when the temperature is 10° C. below Tm. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore, medium stringency hybridisation conditions may sometimes be needed to identify such nucleic acid molecules.

The “Tm” is the temperature under defined ionic strength and pH, at which 50% of the target sequence hybridises to a perfectly matched probe. The Tm is dependent upon the solution conditions and the base composition and length of the probe. For example, longer sequences hybridise specifically at higher temperatures. The maximum rate of hybridisation is obtained from about 16° C. up to 32° C. below Tm. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:
DNA-DNA hybrids (Meinkoth and Wahl, Anal. Biochem., 138:267-284, 1984):
Tm=81.5° C.+16.6×log [Na+]a+0.41×% [G/Cb]−500×[Lc]-1-0.61× % formamide DNA-RNA or RNA-RNA hybrids:
Tm=79.8+18.5 (log 10 [Na+]a)+0.58 (% G/Cb)+11.8 (% G/Cb) 2-820/Lc oligo-DNA or oligo-RNAd hybrids:
For <20 nucleotides: Tm=2(In)
For 20-35 nucleotides: Tm=22+1.46(In)
a or for other monovalent cation, but only accurate in the 0.01-0.4 M range.
b only accurate for % GC in the 30% to 75% range.
c L=length of duplex in base pairs.
d Oligo, oligonucleotide; In, effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For nonrelated probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1× SSC or at 42° C. in 1× SSC and 50% formamide, followed by washing at 65° C. in 0.3× SSC. Examples of medium stringency hybridisation conditions for DNA hybrids longer than 50 nucleotides encompass hybridisation at 50° C. in 4× SSC or at 40° C. in 6× SSC and 50% formamide, followed by washing at 50° C. in 2× SSC. The length of the hybrid is the anticipated length for the hybridising nucleic acid. When nucleic acids of known sequence are hybridised, the hybrid length may be determined by aligning the sequences and identifying the conserved regions described herein. 1× SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate. Another example of high stringency conditions is hybridisation at 65° C. in 0.1× SSC comprising 0.1 SDS and optionally 5×Denhardt's reagent, 100 g/ml denatured, fragmented salmon sperm DNA, 0.5% sodium pyrophosphate, followed by the washing at 65° C. in 0.3× SSC.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

InDel is a term for the random insertion or deletion of bases in the genome of an organism associated with the repair of a DSB by NHEJ. It is classified among small genetic variations, measuring from 1 to 10 000 base pairs in length. As used herein it refers to random insertion or deletion of bases in or in the close vicinity (e.g., less than 1000 bp, 900 bp, 800 bp, 700 bp, 600 bp, 500 bp, 400 bp, 300 bp, 250 bp, 200 bp, 150 bp, 100 bp, 50 bp, 40 bp, 30 bp, 25 bp, 20 bp, 15 bp, 10 bp or 5 bp up and/or downstream) of the target site.

The term “Introducing”, “introduction” and the like with respect to the introduction of a donor DNA molecule in the target site of a target DNA means any introduction of the sequence of the donor DNA molecule into the target region for example by the physical integration of the donor DNA molecule or a part thereof into the target region or the introduction of the sequence of the donor DNA molecule or a part thereof into the target region wherein the donor DNA is used as template for a polymerase.

Intron: refers to sections of DNA (intervening sequences) within a gene that do not encode part of the protein that the gene produces, and that is spliced out of the mRNA that is transcribed from the gene before it is exported from the cell nucleus. Intron sequence refers to the nucleic acid sequence of an intron. Thus, introns are those regions of DNA sequences that are transcribed along with the coding sequence (exons) but are removed during the formation of mature mRNA. Introns can be positioned within the actual coding region or in either the 5′ or 3′ untranslated leaders of the pre-mRNA (unspliced mRNA). Introns in the primary transcript are excised and the coding sequences are simultaneously and precisely ligated to form the mature mRNA. The junctions of introns and exons form the splice site. The sequence of an intron begins with GU and ends with AG. Furthermore, in plants, two examples of AU-AC introns have been described: the fourteenth intron of the RecA-like protein gene and the seventh intron of the G5 gene from Arabidopsis thaliana are AT-AC introns. Pre-mRNAs containing introns have three short sequences that are—beside other sequences-essential for the intron to be accurately spliced. These sequences are the 5′ splice-site, the 3′ splice-site, and the branchpoint. mRNA splicing is the removal of intervening sequences (introns) present in primary mRNA transcripts and joining or ligation of exon sequences. This is also known as cis-splicing which joins two exons on the same RNA with the removal of the intervening sequence (intron). The functional elements of an intron is comprising sequences that are recognized and bound by the specific protein components of the spliceosome (e.g., splicing consensus sequences at the ends of introns). The interaction of the functional elements with the spliceosome results in the removal of the intron sequence from the premature mRNA and the rejoining of the exon sequences. Introns have three short sequences that are essential—although not sufficient—for the intron to be accurately spliced. These sequences are the 5′ splice site, the 3′ splice site and the branch point. The branchpoint sequence is important in splicing and splice-site selection in plants. The branchpoint sequence is usually located 10-60 nucleotides upstream of the 3′ splice site.

Isolated: The term “isolated” as used herein means that a material has been removed by the hand of man and exists apart from its original, native environment and is therefore not a product of nature. An isolated material or molecule (such as a DNA molecule or enzyme) may exist in a purified form or may exist in a non-native environment such as, for example, in a transgenic host cell. For example, a naturally occurring polynucleotide or polypeptide present in a living plant is not isolated, but the same polynucleotide or polypeptide, separated from some or all of the coexisting materials in the natural system, is isolated. Such polynucleotides can be part of a vector and/or such polynucleotides or polypeptides could be part of a composition and would be isolated in that such a vector or composition is not part of its original environment. Preferably, the term “isolated” when used in relation to a nucleic acid molecule, as in “an isolated nucleic acid sequence” refers to a nucleic acid sequence that is identified and separated from at least one contaminant nucleic acid molecule with which it is ordinarily associated in its natural source. Isolated nucleic acid molecule is nucleic acid molecule present in a form or setting that is different from that in which it is found in nature. In contrast, non-isolated nucleic acid molecules are nucleic acid molecules such as DNA and RNA, which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs, which encode a multitude of proteins. However, an isolated nucleic acid sequence comprising for example SEQ ID NO: 1 includes, by way of example, such nucleic acid sequences in cells which ordinarily contain SEQ ID NO:1 where the nucleic acid sequence is in a chromosomal or extrachromosomal location different from that of natural cells or is otherwise flanked by a different nucleic acid sequence than that found in nature. The isolated nucleic acid sequence may be present in single-stranded or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

Non-coding: The term “non-coding” refers to sequences of nucleic acid molecules that do not encode part or all of an expressed protein. Non-coding sequences include but are not limited to introns, enhancers, promoter regions, 3′ untranslated regions, and 5′ untranslated regions.

Nucleic acid expression enhancing nucleic acid (NEENA): The term “nucleic acid expression enhancing nucleic acid” refers to a sequence and/or a nucleic acid molecule of a specific sequence having the intrinsic property to enhance expression of a nucleic acid under the control of a promoter to which the NEENA is functionally linked. Unlike promoter sequences, the NEENA as such is not able to drive expression. In order to fulfil the function of enhancing expression of a nucleic acid molecule functionally linked to the NEENA, the NEENA itself has to be functionally linked to a promoter. In distinction to enhancer sequences known in the art, the NEENA is acting in cis but not in trans and has to be located close to the transcription start site of the nucleic acid to be expressed.

Nucleic acids and nucleotides: The terms “nucleic Acids” and “nucleotides” refer to naturally occurring or synthetic or artificial nucleic acid or nucleotides. The terms “nucleic acids” and “nucleotides” comprise deoxyribonucleotides or ribonucleotides or any nucleotide analogue and polymers or hybrids thereof in either single- or double-stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used interchangeably herein with “gene”, “cDNA, “mRNA”, “oligonucleotide,” and “polynucleotide”. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH2, NHR, NR2, or CN. Short hairpin RNAs (shRNAs) also can comprise nonnatural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

Nucleic acid sequence: The phrase “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid, which is transcribed and translated in a sequence-specific manner to produce into a particular polypeptide or protein when placed under the control of appropriate regulatory sequences. The coding region is said to encode such a polypeptide or protein.

Nucleic acid molecule: The term “nucleic acid molecule” means a molecule comprising “nucleic acids” and “nucleotides”. The term “nucleic acid molecule” refers preferably to small nucleic acid molecules, an oligomer or polymer of ribonucleic acid (RNA) or deoxyribonucleic acid (DNA) or mimetics thereof, as well as small nucleic acid molecules, polynucleotides or oligonucleotides having non-naturally occurring portions which function similarly. Such modified or substituted small nucleic acid molecules, poly- or oligonucleotides are often preferred over native forms because of desirable properties such as, for example, enhanced cellular uptake, enhanced affinity for nucleic acid target and increased stability in the presence of nucleases. A small nucleic acid molecule, poly- or oligonucleotide preferably includes two or more nucleomonomers covalently coupled to each other by linkages (e.g., phosphodiesters) or substitute linkages. For example, the term “nucleic acid molecule” refers to DNA or RNA molecules, without being limited to a particular “nucleic acid sequence”. For example, the term “nucleic acid molecule” relates to molecules comprising sequences of naturally occurring nucleic acid molecules or nucleotides as well as modifications thereof. The term comprises molecules comprising natural occurring sequences as well modified sequences, e.g. by a method described herein. In one embodiment, the nucleic acid molecule is an oligonucleotide, or a polynucleotide.

Oligonucleotide: The term “oligonucleotide” refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages.

Overhang: An “overhang” is a relatively short single-stranded nucleotide sequence on the 5′- or 3′-hydroxyl end of a double-stranded oligonucleotide molecule (also referred to as an “extension,” “protruding end,” or “sticky end”).

Plant: is generally understood as meaning any eukaryotic single- or multi-celled organism or a cell, tissue, organ, part or propagation material (such as seeds or fruit) of same which is capable of photosynthesis. Included for the purpose of the invention are all genera and species of higher and lower plants of the Plant Kingdom. Annual, perennial, monocotyledonous and dicotyledonous plants are preferred. The term includes the mature plants, seed, shoots and seedlings and their derived parts, propagation material (such as seeds or microspores), plant organs, tissue, protoplasts, callus and other cultures, for example cell cultures, and any other type of plant cell grouping to give functional or structural units. Mature plants refer to plants at any desired developmental stage beyond that of the seedling. Seedling refers to a young immature plant at an early developmental stage. Annual, biennial, monocotyledonous and dicotyledonous plants are preferred host organisms for the generation of transgenic plants. The expression of genes is furthermore advantageous in all ornamental plants, useful or ornamental trees, flowers, cut flowers, shrubs or lawns. Plants which may be mentioned by way of example but not by limitation are angiosperms, bryophytes such as, for example, hepaticae (liverworts) and musci (mosses); Pteridophytes such as ferns, horsetail and club mosses; gymnosperms such as conifers, cycads, ginkgo and Gnetatae; algae such as Chlorophyceae, Phaeophpyceae, Rhodophyceae, Myxophyceae, Xanthophyceae, Bacillariophyceae (diatoms), and Euglenophyceae. Preferred are plants which are used for food or feed purpose such as the families of the Leguminosae such as pea, alfalfa and soya; Gramineae such as rice, maize, wheat, barley, sorghum, millet, rye, triticale, or oats; the family of the Umbelliferae, especially the genus Daucus, very especially the species carota (carrot) and Apium, very especially the species Graveolens dulce (celery) and many others; the family of the Solanaceae, especially the genus Lycopersicon, very especially the species esculentum (tomato) and the genus Solanum, very especially the species tuberosum (potato) and melongena (egg plant), and many others (such as tobacco); and the genus Capsicum, very especially the species annuum (peppers) and many others; the family of the leguminosae, especially the genus Glycine, very especially the species max (soybean), alfalfa, pea, lucerne, beans or peanut and many others; and the family of the Cruciferae (Brassicaceae), especially the genus Brassica, very especially the species napus (oil seed rape), campestris (beet), oleracea cv Tastie (cabbage), oleracea cv Snowball Y (cauliflower) and oleracea cv Emperor (broccoli); and of the genus Arabidopsis, very especially the species thaliana and many others; the family of the Compositae, especially the genus Lactuca, very especially the species sativa (lettuce) and many others; the family of the Asteraceae such as sunflower, Tagetes, lettuce or Calendula and many other; the family of the Cucurbitaceae such as melon, pumpkin/squash or zucchini, and linseed. Further preferred are cotton, sugar cane, hemp, flax, chillies, and the various tree, nut and wine species.

Polypeptide: The terms “polypeptide”, “peptide”, “oligopeptide”, “polypeptide”, “gene product”, “expression product” and “protein” are used interchangeably herein to refer to a polymer or oligomer of consecutive amino acid residues.

Pre-protein: Protein, which is normally targeted to a cellular organelle, such as a chloroplast, and still comprising its transit peptide.

“Precise” with respect to the introduction of a donor DNA molecule in target region means that the sequence of the donor DNA molecule is introduced into the target region without any InDels, duplications or other mutations as compared to the unaltered DNA sequence of the target region that are not comprised in the donor DNA molecule sequence.

Primary transcript: The term “primary transcript” as used herein refers to a premature RNA transcript of a gene. A “primary transcript” for example still comprises introns and/or is not yet comprising a polyA tail or a cap structure and/or is missing other modifications necessary for its correct function as transcript such as for example trimming or editing.

Promoter: The terms “promoter”, or “promoter sequence” are equivalents and as used herein, refer to a DNA sequence which when ligated to a nucleotide sequence of interest is capable of controlling the transcription of the nucleotide sequence of interest into RNA. Such promoters can for example be found in the following public databases http://www.grassius.org/grasspromdb.html, http://mendel.cs.rhul.ac.uk/mendel.php?topic=plantprom, http://ppdb.gene.nagoya-u.ac.jp/cgi-bin/index.cgi. Promoters listed there may be addressed with the methods of the invention and are herewith included by reference. A promoter is located 5′ (i.e., upstream), proximal to the transcriptional start site of a nucleotide sequence of interest whose transcription into mRNA it controls, and provides a site for specific binding by RNA polymerase and other transcription factors for initiation of transcription. Said promoter comprises for example the at least 10 kb, for example 5 kb or 2 kb proximal to the transcription start site. It may also comprise the at least 1500 bp proximal to the transcriptional start site, preferably the at least 1000 bp, more preferably the at least 500 bp, even more preferably the at least 400 bp, the at least 300 bp, the at least 200 bp or the at least 100 bp. In a further preferred embodiment, the promoter comprises the at least 50 bp proximal to the transcription start site, for example, at least 25 bp. The promoter does not comprise exon and/or intron regions or 5′ untranslated regions. The promoter may for example be heterologous or homologous to the respective plant. A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g., a genetically engineered coding sequence or an allele from a different ecotype or variety). Suitable promoters can be derived from genes of the host cells where expression should occur or from pathogens for this host cells (e.g., plants or plant pathogens like plant viruses). A plant specific promoter is a promoter suitable for regulating expression in a plant. It may be derived from a plant but also from plant pathogens or it might be a synthetic promoter designed by man. If a promoter is an inducible promoter, then the rate of transcription increases in response to an inducing agent. Also, the promoter may be regulated in a tissue-specific or tissue preferred manner such that it is only or predominantly active in transcribing the associated coding region in a specific tissue type(s) such as leaves, roots or meristem. The term “tissue specific” as it applies to a promoter refers to a promoter that is capable of directing selective expression of a nucleotide sequence of interest to a specific type of tissue (e.g., petals) in the relative absence of expression of the same nucleotide sequence of interest in a different type of tissue (e.g., roots). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter, which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., GUS activity staining, GFP protein or immunohistochemical staining. The term “constitutive” when made in reference to a promoter or the expression derived from a promoter means that the promoter is capable of directing transcription of an operably linked nucleic acid molecule in the absence of a stimulus (e.g., heat shock, chemicals, light, etc.) in the majority of plant tissues and cells throughout substantially the entire lifespan of a plant or part of a plant. Typically, constitutive promoters are capable of directing expression of a transgene in substantially any cell and any tissue.

Promoter specificity: The term “specificity” when referring to a promoter means the pattern of expression conferred by the respective promoter. The specificity describes the tissues and/or developmental status of a plant or part thereof, in which the promoter is conferring expression of the nucleic acid molecule under the control of the respective promoter. Specificity of a promoter may also comprise the environmental conditions, under which the promoter may be activated or down-regulated such as induction or repression by biological or environmental stresses such as cold, drought, wounding or infection.

Purified: As used herein, the term “purified” refers to molecules, either nucleic or amino acid sequences that are removed from their natural environment, isolated or separated. “Substantially purified” molecules are at least 60% free, preferably at least 75% free, and more preferably at least 90% free from other components with which they are naturally associated. A purified nucleic acid sequence may be an isolated nucleic acid sequence.

Recombinant: The term “recombinant” with respect to nucleic acid molecules refers to nucleic acid molecules produced by recombinant DNA techniques. Recombinant nucleic acid molecules may also comprise molecules, which as such does not exist in nature but are modified, changed, mutated or otherwise manipulated by man. Preferably, a “recombinant nucleic acid molecule” is a non-naturally occurring nucleic acid molecule that differs in sequence from a naturally occurring nucleic acid molecule by at least one nucleic acid. A “recombinant nucleic acid molecule” may also comprise a “recombinant construct” which comprises, preferably operably linked, a sequence of nucleic acid molecules not naturally occurring in that order. Preferred methods for producing said recombinant nucleic acid molecule may comprise cloning techniques, directed or non-directed mutagenesis, synthesis or recombination techniques.

Significant increase or decrease: An increase or decrease, for example in enzymatic activity or in gene expression, that is larger than the margin of error inherent in the measurement technique, preferably an increase or decrease by about 2-fold or greater of the activity of the control enzyme or expression in the control cell, more preferably an increase or decrease by about 5-fold or greater, and most preferably an increase or decrease by about 10-fold or greater.

Small nucleic acid molecules: “small nucleic acid molecules” are understood as molecules consisting of nucleic acids or derivatives thereof such as RNA or DNA. They may be double-stranded or single-stranded and are between about 15 and about 30 bp, for example between 15 and 30 bp, more preferred between about 19 and about 26 bp, for example between 19 and 26 bp, even more preferred between about 20 and about 25 bp for example between 20 and 25 bp. In an especially preferred embodiment, the oligonucleotides are between about 21 and about 24 bp, for example between 21 and 24 bp. In a most preferred embodiment, the small nucleic acid molecules are about 21 bp and about 24 bp, for example 21 bp and 24 bp.

Substantially complementary: In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, more desirably at least 70%, more desirably at least 80% or 85%, preferably at least 90%, more preferably at least 93%, still more preferably at least 95% or 96%, yet still more preferably at least 97% or 98%, yet still more preferably at least 99% or most preferably 100% (the latter being equivalent to the term “identical”in this context). Preferably identity is assessed over a length of at least 19 nucleotides, preferably at least 50 nucleotides, more preferably the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J Mol. Biol. 48:443-453; as defined above). A nucleotide sequence “substantially complementary” to a reference nucleotide sequence hybridizes to the reference nucleotide sequence under low stringency conditions, preferably medium stringency conditions, most preferably high stringency conditions (as defined above).

“Target region” as used herein means the region close to, for example 10 bases, 20 bases, 30 bases, 40 bases, 50 bases, 60 bases, 70 bases, 80 bases, 90 bases, 100 bases, 125 bases, 150 bases, 200 bases or 500 bases or more away from the target site, or including the target site in which the sequence of the donor DNA molecule is introduced into the genome of a cell.

“Target site” as used herein means the position in the genome at which a double strand break or one or a pair of single strand breaks (nicks) are induced using recombinant technologies such as Zn-finger, TALEN, restriction enzymes, homing endonucleases, RNA-guided nucleases, RNA-guided nickases such as CRISPR/Cas nucleases or nickases and the like.

Transgene: The term “transgene” as used herein refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

Transgenic: The term transgenic when referring to an organism means transformed, preferably stably transformed, with a recombinant DNA molecule that preferably comprises a suitable promoter operatively linked to a DNA sequence of interest.

Vector: As used herein, the term “vector” refers to a nucleic acid molecule capable of transporting another nucleic acid molecule to which it has been linked. One type of vector is a genomic integrated vector, or “integrated vector”, which can become integrated into the chromosomal DNA of the host cell. Another type of vector is an episomal vector, i.e., a nucleic acid molecule capable of extra-chromosomal replication. Vectors capable of directing the expression of genes to which they are operatively linked are referred to herein as “expression vectors”. In the present specification, “plasmid” and “vector” are used interchangeably unless otherwise clear from the context. Expression vectors designed to produce RNAs as described herein in vitro or in vivo may contain sequences recognized by any RNA polymerase, including mitochondrial RNA polymerase, RNA pol I, RNA pol II, and RNA pol III. These vectors can be used to transcribe the desired RNA molecule in the cell according to this invention. A plant transformation vector is to be understood as a vector suitable in the process of plant transformation.

Wild-type: The term “wild-type”, “natural” or “natural origin” means with respect to an organism, polypeptide, or nucleic acid sequence, that said organism is naturally occurring or available in at least one naturally occurring organism which is not changed, mutated, or otherwise manipulated by man.

According to one embodiment of the present invention, the desired nucleic acid sequence is an RNA sequence. Consequently, the method of the invention comprises steps that allow the analysis of an RNA sequence. For example, RNA molecules are extracted, and the frequency of the desired nucleic acid sequence is determined in the RNA molecules.

The method of the invention can also comprise extracting RNA or RNA and DNA molecules and producing DNA molecules from the extracted RNA molecules, for example with a reverse transcription method. The produced DNA molecules, or the produced DNA molecules and extracted DNA molecules, are analysed to determine the quantity of the desired nucleic acid sequence. For example, the RNA is extracted from the regenerative cells or cultured callus or regenerated shoots or any other material that is to be analysed according to the method of the invention.

### Tables:

TABLE 1
ddPCR mlo drop-off percentages for
immature embryos from TMTA0233

| | | | | ddPCR |
|---|---|---|---|---|
| Shot | Rupture Disk | Shooting | | drop-off |
| Number | Pressure (psi) | Distance | Sample Number | (%) |
| 1 | 650 | L2 (2 cm) | TMTA0233-B01$001 | 5.6 |
| 2 | 650 | L2 (2 cm) | TMTA0233-B02$001 | 4.0 |
| 3 | 650 | L2 (2 cm) | TMTA0233-B03$001 | 3.4 |
| 4 | 1350 | L2 (2 cm) | TMTA0233-B04$001 | 3.4 |
| 5 | 1350 | L2 (2 cm) | TMTA0233-B05$001 | 5.0 |
| 6 | 1350 | L3 (5 cm) | TMTA0233-B06$001 | 2.0 |

TABLE 2
Summary of mlo ddPCR results obtained
for pooled leaf samples from TMTA0233

| ddPCR drop−off % | Shot 1 | | Shot 2 | | Shot 5 | |
|---|---|---|---|---|---|---|
| category | # pools | % pools | # pools | % pools | # pools | % pools |
| <10% | 4 | 6.3 | 29 | 37.2 | 6 | 18.8 |
| 10−25% | 19 | 30.2 | 24 | 30.8 | 11 | 34.4 |
| 25−50% | 27 | 42.9 | 16 | 20.5 | 13 | 40.6 |
| >50% | 13 | 20.6 | 9 | 11.5 | 2 | 6.2 |
| Total | 63 | 100 | 78 | 100 | 32 | 100 |

TABLE 3
Summary of mlo ddPCR results obtained
for individual shoots from TMTA0233

| | Pool % | # of individual | % drop-off | | |
|---|---|---|---|---|---|
| Pool reference | drop-off | plants sampled | 0% | 50% | 100% |
| TMTA0233-038-B01 | 2 | 23 | 20 | 3 | 0 |
| TMTA0233-045-B03 | 7 | 7 | 5 | 2 | 0 |
| TMTA0233-051-B04 | 11 | 21 | 17 | 3 | 1 |
| TMTA0233-051-B01 | 22 | 12 | 9 | 1 | 2 |
| TMTA0233-041-B01 | 23 | 11 | 6 | 5 | 0 |
| TMTA0233-033-B01 | 24 | 12 | 9 | 0 | 3 |
| TMTA0233-049-B01 | 21 | 10 | 4 | 4 | 2 |
| TMTA0233-038-B04 | 25 | 11 | 7 | 1 | 3 |
| TMTA0233-084-B04 | 24 | 24 | 13 | 8 | 3 |
| TMTA0233-036-B01 | 40 | 5 | 1 | 0 | 4 |
| TMTA0233-050-B01 | 41 | 5 | 2 | 1 | 2 |
| TMTA0233-044-B04 | 43 | 5 | 4 | 0 | 1 |
| TMTA0233-035-B01 | 46 | 6 | 2 | 1 | 3 |
| TMTA0233-047-B04 | 48 | 5 | 1 | 2 | 2 |
| TMTA0233-032-B02 | 50 | 14 | 3 | 6 | 5 |
| TMTA0233-050-B04 | 50 | 12 | 5 | 1 | 6 |
| TMTA0233-047-B03 | 51 | 17 | 11 | 1 | 5 |
| TMTA0233-035-B02 | 52 | 13 | 6 | 4 | 3 |
| TMTA0233-044-B03 | 52 | 16 | 7 | 4 | 5 |
| TMTA0233-047-B05 | 53 | 15 | 8 | 3 | 4 |
| TMTA0233-046-B02 | 54 | 29 | 10 | 8 | 11 |
| TMTA0233-048-B03 | 54 | 18 | 11 | 2 | 5 |
| TMTA0233-047-B01 | 56 | 22 | 9 | 3 | 10 |
| TMTA0233-035-B04 | 57 | 17 | 8 | 1 | 8 |
| TMTA0233-032-B01 | 58 | 32 | 10 | 13 | 9 |
| TMTA0233-035-B03 | 58 | 17 | 5 | 4 | 8 |
| TMTA0233-037-B01 | 58 | 25 | 2 | 14 | 9 |
| TMTA0233-037-B02 | 62 | 11 | 7 | 0 | 4 |
| TMTA0233-052-B01 | 81 | 11 | 0 | 0 | 11 |

TABLE 4
Summary of mlo NGS results obtained for individual analyzed shoots from TMTA0233.

| | % drop- | | | | | | | | | | | | |
|---|---|---|---|---|---|---|---|---|---|---|---|---|---|
| | off | | | | | | | | | | | | |
| | Single | A-Genome | | | | B-Genome | | | | D-genome | | | |
| Single Plant Sample | Plant | allele 1 | | allele 2 | | allele 1 | | allele 2 | | allele 1 | | allele 2 | |
| TMTA0233-032-B01-01$001 | 52 | +1 | bp | −5 | bp | +1 | bp | +1 | bp/indel | −1 | bp | −3 | bp |
| TMTA0233-032-B01-06$001 | 100 | −4 | bp | −5 | bp (1SNP) | +1 | bp | −4 | bp | −1 | bp | −2 | bp |
| TMTA0233-032-B01-08$001 | 0 | WT | | WT | | WT | | WT | | | | WT | |
| TMTA0233-033-B01-08$001 | 100 | −5 | bp | −6 | bp | +1 | bp | −4 | bp | −4 | bp | −5 | bp |
| TMTA0233-035-B01-03$001 | 50 | +1 | bp | −62 | bp | WT | | WT | | −2 | bp | −5 | bp |
| TMTA0233-035-B02-05$001 | 0 | +1 | bp | WT | | +1 | bp | +1 | bp/indel | −3 | bp | WT | |
| TMTA0233-035-B02-06$001 | 100 | −1 | bp | −3 | bp | +1 | bp | WT | | ND | | ND | |
| TMTA0233-035-B03-07$001 | 52 | −5 | bp | WT | | −6 | bp | WT | | −1 | bp | −4 | bp |
| TMTA0233-035-B04-01$001 | 100 | −5 | bp | −5 | bp/indel | −4 | bp | −4 | bp/indel | +1 | bp | +1 | bp/indel |
| TMTA0233-035-B04-02$001 | 0 | +1 | bp | WT | | +1 | bp | +1 | bp/indel | −3 | bp | WT | |
| TMTA0233-035-B04-04$001 | 0 | +1 | bp | +1 | bp/indel | −5 | bp | WT | | +1 | bp | −2 | bp |
| TMTA0233-035-B04-12$001 | 100 | +1 | bp | +1 | bp/indel | −1 | bp (1SNP) | −5 | bp | −1 | bp | −3 | bp |
| TMTA0233-035-B04-16$001 | 52 | −68 | bp | WT | | WT | | WT | | WT | | −1 | bp |
| TMTA0233-036-B01-01$001 | 0 | WT | | WT | | WT | | WT | | WT | | WT | |
| TMTA0233-038-B04-01$001 | 100 | −3 | bp | −29 | bp | WT | | WT | | WT | | WT | |
| TMTA0233-038-B04-05$001 | 51 | −5 | bp | WT | | −2 | bp | WT | | −1 | bp | −2 | bp |
| TMTA0233-044-B03-13$001 | 100 | −6 | bp | −9 | bp | −1 | bp | −4 | bp | −2 | bp | −5 | bp |
| TMTA0233-044-B04-03$001 | 1 | WT | | WT | | WT | | WT | | WT | | WT | |
| TMTA0233-044-B04-06$001 | 12 | WT | | WT/indel | | WT | | −1 | bp/indel | WT | | WT | |
| TMTA0233-046-B02-04$001 | 100 | (1SNP) | | −5 | bp (1SNP) | −4 | bp | −4 | bp/indel | −1 | bp | WT | |
| TMTA0233-046-B02-24$001 | 1 | WT | | WT | | WT | | WT | | WT | | WT | |
| TMTA0233-047-B01-01$001 | 100 | −2 | bp | −10 | bp | +1 | bp | WT | | +1 | bp | −2 | bp |
| TMTA0233-047-B04-01$001 | 52 | −5 | bp | WT | | WT | | WT | | −1 | bp | −3 | bp (1SNP) |
| TMTA0233-047-B04-04$001 | 99 | ND | | ND | | +1 | bp | +1 | bp/indel | −1 | bp | −2 | bp |
| TMTA0233-048-B03-17$001 | 100 | −1 | bp (6 SNP) | −35 | bp | WT | | WT | | −3 | bp | WT | |
| TMTA0233-048-B03-18$001 | 100 | ND | | ND | | −5 | bp (1SNP) | −5 | bp (1SNP)/indel | −3 | bp | −3 | bp/indel |
| TMTA0233-050-B01-01$001 | 100 | −2 | bp | −38 | bp | −3 | bp | −9 | bp (4 SNP) | −2 | bp | −4 | bp |

| TMTA0233-050-B01-05$001 | 51 | +1 | bp | −3 | bp | WT | | WT | +1 | bp | −2 | bp |
|---|---|---|---|---|---|---|---|---|---|---|---|---|
| TMTA0233-052-B01-01$001 | 100 | −1 | bp | −11 | bp | −1 | bp | WT | −3 | bp | −7 | bp |

Note:
Some of the individual alleles for the A, B and D mlo suB-genomes could not be fully profiled e.g. +1 bp/indel. Such plants are either biallelic mutants or alternatively, one of the alleles carries a large indel sequence, which interferes with the NGS analysis. Shaded cells indicate plants with unique mutation profiles.

TABLE 5
Summary of mlo NGS results obtained for individual shoots
originating from one individual embryo explant of TMTA0233

| | % drop−off | A-Genome | | | | B-Genome | | | | D-genome | | | |
|---|---|---|---|---|---|---|---|---|---|---|---|---|---|
| Single Plant Sample | Single Plant | allele 1 | | allele 2 | | allele 1 | | allele 2 | | allele 1 | | allele 2 | |
| TMTA0233-037-B02-03$001 | 0 | WT | | WT | | WT | | WT | | WT | | WT | |
| TMTA0233-037-B02-11$001 | 0 | WT | | WT | | WT | | WT | | WT | | WT | |
| TMTA0233-037-B02-12$001 | 0 | WT | | WT | | WT | | WT | | WT | | WT | |
| TMTA0233-037-B02-13$001 | 0 | WT | | WT | | WT | | WT | | WT | | WT | |
| TMTA0233-037-B01-11$001 | 1 | WT | | WT | | WT | | WT | | −1 | bp | WT | |
| TMTA0233-037-B01-01$001 | 51 | +1 | bp | −4 | bp | +1 | bp | WT | | −3 | bp | −5 | bp |
| TMTA0233-037-B01-09$001 | 50 | +1 | bp | −4 | bp | +1 | bp | WT | | −3 | bp | −5 | bp |
| TMTA0233-037-B01-10$001 | 50 | +1 | bp | −4 | bp | +1 | bp | WT | | −3 | bp | −5 | bp |
| TMTA0233-037-B01-03$001 | 52 | +1 | bp | −2 | bp | −4 | bp | −4 | bp/indel | −2 | bp | −3 | bp |
| TMTA0233-037-B01-08$001 | 60 | +1 | bp | −2 | bp | −4 | bp | −4 | bp/indel | −2 | bp | −3 | bp |
| TMTA0233-037-B01-13$001 | 50 | +1 | bp | −2 | bp | −4 | bp | WT | | −2 | bp | −3 | bp |
| TMTA0233-037-B01-17$001 | 51 | +1 | bp | −2 | bp | −4 | bp | +1 | bp | −2 | bp | −3 | bp |
| TMTA0233-037-B01-20$001 | 51 | +1 | bp | −9 | bp | −1 | bp | −7 | bp | −14 | bp | −14 | bp/indel |
| TMTA0233-037-B02-05$001 | 25 | +1 | bp | −3 | bp | +1 | bp | +1 | bp/indel | +1 | bp | −10 | bp |
| TMTA0233-037-B02-06$001 | 64 | +1 | bp | −3 | bp | +1 | bp | +1 | bp/indel | −10 | bp | WT | |
| TMTA0233-037-B02-10$001 | 30 | +1 | bp | −3 | bp | +1 | bp | +1 | bp/indel | −10 | bp | WT | |
| TMTA0233-037-B01-06$001 | 100 | −3 | bp | −10 | bp | +1 | bp | WT | | −4 | bp | WT | |
| TMTA0233-037-B01-14$001 | 100 | −3 | bp | −10 | bp | +1 | bp | WT | | −4 | bp | WT | |
| TMTA0233-037-B01-15$001 | 100 | −5 | bp | −5 | bp/indel | −1 | bp | WT | | −4 | bp | −5 | bp |
| TMTA0233-037-B01-18$001 | 100 | −2 | bp | −5 | bp | +1 | bp | −4 | bp | −4 | bp | −3 | bp |
| TMTA0233-037-B01-22$001 | 100 | −6 | bp | −6 | bp/indel | +1 | bp | WT | | +1 | bp | −1 | bp |
| TMTA0233-037-B01-25$001 | 100 | −2 | bp | −5 | bp | +1 | bp | −29 | bp | +1 | bp | −2 | bp |

| | | | | | | | | | | |
|---|---|---|---|---|---|---|---|---|---|---|
| TMTA0233-037-B02-07$001 | 99 | −1 | bp | −3 | bp | WT | WT | −3 | bp | WT |

Note:
Some of the individual alleles for the A, B and D mlo suB-genomes could not be fully profiled e.g. +1 bp/indel. Such plants are either biallelic mutants or alternatively, one of the alleles carries a large indel sequence, which interferes with the NGS analysis. Shaded cells indicate plants with unique mutation pro-files.

TABLE 6
ddPCR mlo drop-off percentages for
immature embryos from TMTA0527

| | | | | ddPCR |
|---|---|---|---|---|
| Shot | Rupture Disk | Shooting | | drop-off |
| Number | Pressure (psi) | Distance | Sample Number | (%) |
| 1 | 650 | L2 (2 cm) | TMTA0527-B01$001 | 1.9 |
| 2 | 650 | L2 (2 cm) | TMTA0527-B02$001 | 2.7 |
| 3 | 650 | L2 (2 cm) | TMTA0527-B03$001 | 0.6 |
| 4 | 650 | L2 (2 cm) | TMTA0527-B04$001 | 0.5 |
| 5 | 650 | L2 (2 cm) | TMTA0527-B05$001 | 1.6 |
| 6 | 650 | L2 (2 cm) | TMTA0527-B06$001 | 0.6 |

TABLE 7
Summary of mlo ddPCR results obtained
for pooled leaf samples from TMTA0527

| ddPCR drop−off % | Shot 1 | | Shot 2 | | Shot 5 | |
|---|---|---|---|---|---|---|
| category | # pools | % pools | # pools | % pools | # pools | % pools |
| <10% | 23 | 39.7 | 7 | 22.6 | 35 | 70 |
| 10−25% | 19 | 32.8 | 6 | 19.4 | 11 | 22 |
| 25−50% | 11 | 19 | 13 | 41.9 | 4 | 8 |
| >50% | 5 | 8.6 | 5 | 16.1 | 0 | 0 |
| Total | 58 | 100 | 31 | 100 | 50 | 100 |

TABLE 8
Summary of mlo ddPCR results obtained
for individual shoots from TMTA0527

| | Pool % | # of individual | % drop-off | | |
|---|---|---|---|---|---|
| Pool reference | drop-off | plants sampled | 0% | 50% | 100% |
| TMTA0527-033-B02 | 16.0 | 21 | 10 | 11 | 0 |
| TMTA0527-036-B03 | 24.1 | 21 | 13 | 8 | 0 |
| TMTA0527-037-B02 | 16.1 | 25 | 20 | 5 | 0 |
| TMTA0527-038-B01 | 14.6 | 24 | 19 | 4 | 1 |
| TMTA0527-039-B04 | 30.8 | 15 | 8 | 7 | 0 |
| TMTA0527-040-B02 | 17.3 | 19 | 18 | 0 | 1 |
| TMTA0527-041-B03 | 37.4 | 20 | 8 | 10 | 2 |
| TMTA0527-043-B05 | 45.2 | 17 | 4 | 11 | 2 |
| TMTA0527-044-B06 | 68.0 | 17 | 3 | 7 | 7 |
| TMTA0527-045-B02 | 52.9 | 18 | 8 | 5 | 5 |
| TMTA0527-045-B03 | 41.2 | 13 | 6 | 4 | 3 |
| TMTA0527-045-B04 | 47.0 | 16 | 6 | 4 | 6 |
| TMTA0527-045-B05 | 41.2 | 15 | 5 | 7 | 3 |
| TMTA0527-049-B06 | 49.5 | 17 | 8 | 5 | 4 |
| TMTA0527-050-B01 | 77.6 | 4 | 0 | 0 | 4 |
| TMTA0527-051-B01 | 49.0 | 17 | 8 | 7 | 2 |
| TMTA0527-052-B02 | 34.0 | 16 | 8 | 7 | 1 |
| TMTA0527-053-B04 | 25.8 | 4 | 1 | 3 | 0 |
| TMTA0527-054-B01 | 44.2 | 14 | 7 | 6 | 1 |
| TMTA0527-057-B02 | 51.0 | 22 | 6 | 9 | 7 |
| TMTA0527-058-B01 | 60.0 | 15 | 3 | 6 | 6 |
| TMTA0527-064-B01 | 36.8 | 8 | 4 | 0 | 4 |
| TMTA0527-067-B02 | 17.5 | 19 | 11 | 4 | 4 |
| TMTA0527-069-B02 | 10.9 | 17 | 11 | 5 | 1 |
| TMTA0527-071-B04 | 34.9 | 18 | 5 | 13 | 0 |
| TMTA0527-074-B01 | 28.0 | 9 | 2 | 7 | 0 |
| TMTA0527-075-B02 | 31.4 | 15 | 10 | 0 | 5 |

TABLE 9
Summary of mlo NGS results obtained for individual analyzed shoots from

| | % drop−off | A-Genome | | | | B-Genome | | | D-genome | | | |
|---|---|---|---|---|---|---|---|---|---|---|---|---|
| Single Plant Sampe | Single Plant | allele 1 | | allele 2 | | allele 1 | | alele 2 | allele 1 | | allele 2 | |
| [symbol] | 100 | −3 | bp | −8 | bp | −4 | bp | WT | −3 | bp | WT | |
| [symbol] | 51 | −10 | bp | WT | | WT | | WT | −9 | bp | WT | |
| [symbol] | 100 | 6 | bp | 6 | bp/ndel | WT | | WT | WT | | WT | |
| [symbol] | 0 | WT | | WT | | WT | | WT | −9 | bp | −9 | bp |
| [symbol] | 0 | WT | | WT | | WT | | WT | −9 | bp | −9 | bp |
| [symbol] | 50 | −11 | bp | WT | | WT | | WT | −3 | bp | WT | |
| [symbol] | 100 | −13 | bp | −32 | bp | WT | bp | WT | −9 | bp | WT | |
| [symbol] | | −3 | bp | WT | | WT | | WT | WT | | WT | |
| [symbol] | 51 | −3 | bp | WT | | WT | | WT | −9 | bp | −10 | bp |
| [symbol] | 100 | −3 | bp | −6 | bp | WT | | WT | WT | | WT | |
| [symbol] | 50 | −17 | bp | WT | | WT | | WT | −11 | bp | [symbol] | bp |
| [symbol] | 50 | −9 | bp | WT | | WT | | WT | −8 | bp | WT | |
| [symbol] | 100 | −8 | bp | −14 | (1 SNP) | −7 | (1 SNP) | WT | −7 | bp | −10 | bp |
| [symbol] | 51 | −9 | bp | WT | | −7 | bp | WT | −3 | bp | WT | |
| [symbol] | 100 | −8 | bp | −14 | (1 SNP) | WT | | WT | [symbol] | | [symbol] | bp |
| [symbol] | 100 | −3 | bp | −5 | bp | −1 | bp | WT | [symbol] | bp | −13 | bp |
| [symbol] | 100 | −6 | bp | −8 | bp | −8 | bp | WT | −9 | bp | −12 | bp |
| [symbol] | 100 | −11 | bp | −14 | bp | −48 | bp | WT | −1.1 | bp | [symbol] | bp |

| | | | | | | | | | | |
|---|---|---|---|---|---|---|---|---|---|---|
| [symbol] | 0 | WT | WT | −3 | bp | −4 | bp | −12 | bp | WT |

| [symbol] | 49 | −1/bp | | WT | WT | WT | −10 | bp | WT |
|---|---|---|---|---|---|---|---|---|---|
| [symbol] | 0.4 | +1 | bp | WT | WT | WT | −9 | bp | WT |

Note:
Some of the individual alleles for the A, B and D mlo suB-genomes could not be fully profiled e.g. −6 bp/indel. Such plants are either biallelic mutants or alternatively, one of the alleles carries a large indel sequence, which interferes with the NGS analysis Shaded cells indicate plants with unique mutation profiles. [symbol]
indicates data missing or illegible when filed

TABLE 10
Summary of mlo NGS results obtained for individual shoots originating from one individual embryo explant of TMTA0527

| | % drop-off | A-Genome | | | | B-Genome | | | D-genome | | | |
|---|---|---|---|---|---|---|---|---|---|---|---|---|
| Single Plant Sample | Single Plant | allele 1 | | allele 2 | | allele 1 | | allele 2 | allele 1 | | allele 2 | |
| TMTA0527-045-B02-01$001 | 0 | WT | | WT | | WT | | WT | WT | | WT | |
| TMTA0527-045-B02-03$001 | 100 | −6 | bp | −7 | bp | −1 | bp | WT | −8 | bp | −8 | bp |
| TMTA0527-045-B02-04$001 | 50 | −8 | bp | WT | | WT | | WT | −3 | bp | −9 | bp |
| TMTA0527-045-B02-06$001 | 49 | −3 | bp | WT | | WT | | WT | −14 | bp | WT | |
| TMTA0527-045-B02-08$001 | 100 | −6 | bp | −7 | bp | −1 | bp | WT | −8 | bp | −8 | bp |

| TMTA0527-045-B02-09$001 | 51 | −9 | bp | WT | | WT | WT | WT | | WT | |
|---|---|---|---|---|---|---|---|---|---|---|---|
| TMTA0527-045-B02-10$001 | 100 | −8 | bp | −8 | bp | WT | WT | −9 | bp | −10 | bp |
| TMTA0527-045-B02-13$001 | 52 | −9 | bp | WT | | WT | WT | −4 | bp | −13 | bp |
| TMTA0527-045-B02-14$001 | 100 | −9 | bp | −9 | bp/indel | WT | WT | −15 | bp | −15 | bp/indel |
| TMTA0527-045-B02-18$001 | 100 | −8 | bp | −11 | bp | WT | WT | +1 | bp | −4 | bp (8 SNP) |
| TMTA0527-045-B03-01$001 | 52 | −15 | bp | WT | | WT | WT | WT | | WT | |
| TMTA0527-045-B03-03$001 | 99 | −7 | bp | −7 | bp/indel | WT | WT | −9 | bp | WT | |
| TMTA0527-045-B03-04$001 | 100 | −10 | bp | −18 | bp | WT | WT | −8 | bp | −22 | bp |

| | | | | | | | | | | |
|---|---|---|---|---|---|---|---|---|---|---|
| TMTA0527-045-B03-06$001 | 50 | −9 | bp | WT | WT | WT | −3 | bp | −9 | bp |

| | | | | | | | | | |
|---|---|---|---|---|---|---|---|---|---|
| TMTA0527-045-B03-07$001 | 0 | WT | WT | −7 | bp | WT | −15 | bp | WT |

| | | | | | | | | | | | |
|---|---|---|---|---|---|---|---|---|---|---|---|
| TMTA0527-045-B03-09$001 | 100 | −7 | bp | −9 | bp | WT | WT | −7 | bp | −15 | bp |

| TMTA0527-045-B03-10$001 | 50 | −8 | bp | WT | −8 | bp | WT | WT | | WT |
|---|---|---|---|---|---|---|---|---|---|---|
| TMTA0527-045-B03-13$001 | 0 | WT | | WT | −7 | bp | WT | −15 | bp | WT |
| TMTA0527-045-B04-01$001 | 49 | −11 | bp | WT | −15 | bp | WT | −9 | bp | WT |

| TMTA0527-045-B04-02$01 | 100 | −10 | bp | −12 | bp | WT | WT | −7 | bp | −9 | bp |
|---|---|---|---|---|---|---|---|---|---|---|---|
| TMTA0527-045-B04-03$001 | 50 | −8 | bp | WT | | WT | WT | WT | | WT | |
| TMTA0527-045-B04-05$001 | 100 | −10 | bp | −18 | bp | WT | WT | −8 | bp | WT | |
| TMTA0527-045-B04-06$001 | 100 | −9 | bp | −9 | bp/indel | WT | WT | −3 | bp | WT | |
| TMTA0527-045-B04-08$001 | 100 | −10 | bp | −12 | bp | WT | WT | −7 | bp | −9 | bp |
| TMTA0527-045-B04-11$001 | 100 | −9 | bp | −13 | bp | WT | WT | −10 | bp | −10 | bp/indel |

| TMTA0527-045-B04-15$001 | 50 | −6 | bp | WT | | WT | WT | −9 | bp | WT |
|---|---|---|---|---|---|---|---|---|---|---|
| TMTA0527-045-B04-16$001 | 0 | WT | | WT | | WT | WT | −9 | bp | WT |
| TMTA0527-045-B05-01$001 | 51 | −10 | bp | WT | | WT | WT | WT | | WT |
| TMTA0527-045-B05-02$001 | 49 | −8 | bp | WT | | WT | WT | −8 | bp | WT |
| TMTA0527-045-B05-03$001 | 100 | −8 | bp | −8 | bp | WT | WT | −9 | bp | WT |

| TMTA0527-045-B05-05$001 | 0.4 | WT | | WT | WT | WT | WT | | WT | |
|---|---|---|---|---|---|---|---|---|---|---|
| TMTA0527-045-B05-09$001 | 50 | −5 | bp | WT | WT | WT | −3 | bp | −5 | bp |
| TMTA0527-045-B05-10$001 | 50 | −15 | bp | WT | WT | WT | −3 | bp | −9 | bp |

| TMTA0527-045-B05-11$001 | 51 | −9 | bp | WT | | WT | | WT | −10 | bp | WT |
|---|---|---|---|---|---|---|---|---|---|---|---|
| TMTA0527-045-B05-14$001 | 100 | −10 | bp | −10 | bp/indel | −14 | bp | WT | −9 | bp | WT |

Note:
Some of the individual alleles for the A, B and D mlo suB-genomes could not be fully profiled e.g. −9 bp/indel. Such plants are either biallelic mutants or alternatively, one of the alleles carries a large indel sequence, which interferes with the NGS analysis. Shaded cells indicate plants with unique mutation
profiles.

TABLE 11
Summary of mlo NGS results obtained for progeny
derived from plant TMTA0233-032-B0-06.

| | A-Genome | | | | B-Genome | | | | D-genome | | | |
|---|---|---|---|---|---|---|---|---|---|---|---|---|
| Seedling | allele 1 | | allele 2 | | allele 1 | | allele 2 | | allele 1 | | allele 2 | |
| 1 | −5 (1 SNP) | | −5 (1 SNP) | | +1 | bp | −4 | bp | −2 | bp | −2 | bp |
| 2 | −4 | bp | −4 | bp | +1 | bp | +1 | bp | 1 | bp | −2 | bp |
| 3 | −4 | bp | 4 | bp | +1 | bp | +1 | bp | −2 | bp | −2 | bp |
| 4 | −5 (1 SNP) | | −4 | bp | +1 | bp | −4 | bp | −1 | bp | −2 | bp |
| 5 | −5 (1 SNP) | | −4 | bp | +1 | bp | +1 | bp | −1 | bp | −1 | bp |
| 6 | −4 | bp | −4 | bp | −4 | bp | −4 | bp | −2 | bp | −2 | bp |
| 7 | −5 (1 SNP) | | −4 | bp | +1 | bp | −4 | bp | −1 | bp | −2 | bp |
| | −5 (1 SNP) | | −4 | bp | +1 | bp | −4 | bp | −2 | bp | −2 | bp |
| 9 | −5 (1 SNP) | | −4 | bp | +1 | bp | +1 | bp | −1 | bp | −1 | bp |
| 10 | −5 (1 SNP) | | −5 (1 SNP) | | −4 | bp | −4 | bp | −1 | bp | −2 | bp |
| 11 | −5 (1 SNP) | | −4 | bp | +1 | bp | −4 | bp | −2 | bp | −2 | bp |
| 12 | −4 | bp | −4 | bp | −4 | bp | −4 | bp | −1 | bp | −2 | bp |
| 13 | −5 (1 SNP) | | −5 (1 SNP) | | −4 | bp | −4 | bp | −1 | bp | −2 | bp |
| 14 | −5 (1 SNP) | | −4 | bp | +1 | bp | −4 | bp | −1 | bp | −1 | bp |
| 15 | −5 (1 SNP) | | −5 (1 SNP) | | +1 | bp | +1 | bp | −2 | bp | −2 | bp |
| 16 | −5 (1 SNP) | | −5 (1 SNP) | | +1 | bp | +1 | bp | −1 | bp | −1 | bp |
| 17 | 4 | bp | −4 | bp | +1 | bp | +1 | bp | −1 | bp | 2 | bp |
| 18 | −5 (1 SNP) | | −5 (1 SNP) | | +1 | bp | −4 | bp | −1 | bp | −2 | bp |
| 19 | −5 (1 SNP) | | −4 | bp | +1 | bp | −4 | bp | −1 | bp | −2 | bp |
| 20 | −5 (1 SNP) | | −5 (1 SNP) | | +1 | bp | −4 | bp | −1 | bp | −2 | bp |
| 21 | −5 (1 SNP) | | −4 | bp | −4 | bp | −4 | bp | −1 | bp | −2 | bp |
| 22 | −5 (1 SNP) | | −4 | bp | +1 | bp | −4 | bp | −2 | bp | −2 | bp |
| 23 | −4 | bp | −4 | bp | +1 | bp | −4 | bp | −2 | bp | −2 | bp |
| 24 | −5 (1 SNP) | | −5 (1 SNP) | | +1 | bp | −4 | bp | −2 | bp | −2 | bp |
| 25 | −4 | bp | −4 | bp | +1 | bp | −4 | bp | −1 | bp | −2 | bp |

TABLE 12
Summary of mlo NGS results obtained for progeny
derived from plant TMTA0233-035-B04-01.

| | A-Genome | | B-Genome | | D-genome | |
|---|---|---|---|---|---|---|
| Seedling | allele 1 | allele 2 | allele 1 | allele 2 | allele 1 | allele 2 |
| 1 | −5 bp | −5 bp | −4 bp | large insert | large insert | large insert |
| 2 | −5 bp | −5 bp | −4 bp | large insert | large insert | large insert |
| 3 | −5 bp | −5 bp | large insert | large insert | +1 bp | large insert |
| 4 | −5 bp | −5 bp | large insert | large insert | +1 bp | +1 bp |
| 5 | −5 bp | −5 bp | −4 bp | −4 bp | +1 bp | large insert |
| 6 | −5 bp | −5 bp | large insert | large insert | +1 bp | large insert |
| 7 | −5 bp | −5 bp | −4 bp | large insert | large insert | large insert |
| 8 | −5 bp | −5 bp | −4 bp | large insert | large insert | large insert |
| 9 | −5 bp | −5 bp | large insert | large insert | +1 bp | large insert |
| 10 | −5 bp | −5 bp | −4 bp | large insert | large insert | large insert |
| 11 | −5 bp | −5 bp | −4 bp | large insert | +1 bp | large insert |
| 12 | −5 bp | −5 bp | −4 bp | large insert | +1 bp | large insert |
| 13 | −5 bp | −5 bp | −4 bp | −4 bp | +1 bp | large insert |
| 14 | −5 bp | −5 bp | −4 bp | large insert | +1 bp | +1 bp |
| 15 | −5 bp | −5 bp | −4 bp | large insert | large insert | large insert |
| 16 | −5 bp | −5 bp | −4 bp | large insert | +1 bp | +1 bp |
| 17 | −5 bp | −5 bp | large insert | large insert | +1 bp | +1 bp |
| 18 | −5 bp | −5 bp | −4 bp | large insert | +1 bp | +1 bp |
| 19 | −5 bp | −5 bp | −4 bp | large insert | +1 bp | +1 bp |
| 20 | −5 bp | −5 bp | −4 bp | −4 bp | +1 bp | large insert |
| 21 | −5 bp | −5 bp | −4 bp | −4 bp | +1 bp | +1 bp |
| 22 | −5 bp | −5 bp | −4 bp | large insert | +1 bp | +1 bp |
| 23 | −5 bp | −5 bp | −4 bp | large insert | +1 bp | large insert |

TABLE 13
Summary of mlo NGS results obtained for progeny
derived from plant TMTA0233-044-B03-13.

| | A-Genome | | B-Genome | | D-genome | |
|---|---|---|---|---|---|---|
| Seedling | allele 1 | allele 2 | allele 1 | allele 2 | allele 1 | allele 2 |
| 1 | −9 bp | −9 bp | −1 bp | −1 bp | −5 bp | −5 bp |
| 2 | −6 bp | −6 bp | −4 bp | −4 bp | −2 bp | −5 bp |
| 3 | −9 bp | −9 bp | −1 bp | −4 bp | −2 bp | −5 bp |
| 4 | −9 bp | −9 bp | 4 bp | −4 bp | −5 bp | −5 bp |
| 5 | −9 bp | −9 bp | −1 bp | −4 bp | −2 bp | −5 bp |
| 6 | −6 bp | −9 bp | −4 bp | −4 bp | −5 bp | −5 bp |
| 7 | −6 bp | −6 bp | −4 bp | −4 bp | −2 bp | −5 bp |
| 8 | −6 bp | −9 bp | −4 bp | −4 bp | −5 bp | −5 bp |
| 9 | −9 bp | −9 bp | −1 bp | −4 bp | −2 bp | −5 bp |
| 10 | −6 bp | −9 bp | −1 bp | −4 bp | −5 bp | −5 bp |
| 11 | −6 bp | −6 bp | −1 bp | −4 bp | −2 bp | −5 bp |
| 12 | −6 bp | −6 bp | −1 bp | −4 bp | −2 bp | −2 bp |
| 13 | −6 bp | −6 bp | −4 bp | −4 bp | −2 bp | −5 bp |
| 14 | −6 bp | −6 bp | −1 bp | −4 bp | −5 bp | −5 bp |
| 15 | −6 bp | −9 bp | −4 bp | −4 bp | −5 bp | −5 bp |
| 16 | −6 bp | −9 bp | −4 bp | −4 bp | −2 bp | −5 bp |
| 17 | −6 bp | −9 bp | −1 bp | −4 bp | −5 bp | −5 bp |
| 18 | −6 bp | −9 bp | −1 bp | −4 bp | −2 bp | −5 bp |
| 19 | −6 bp | −9 bp | −1 bp | −4 bp | −2 bp | −5 bp |
| 20 | −6 bp | −9 bp | −1 bp | −4 bp | −5 bp | −5 bp |
| 21 | −9 bp | −9 bp | −1 bp | −4 bp | −2 bp | −2 bp |
| 22 | −9 bp | −9 bp | −1 bp | −4 bp | −5 bp | −5 bp |
| 23 | −6 bp | −6 bp | −1 bp | −4 bp | −2 bp | −5 bp |
| 24 | −6 bp | −9 bp | −4 bp | −4 bp | −2 bp | −2 bp |
| 25 | −6 bp | −9 bp | −1 bp | −4 bp | −2 bp | −5 bp |
| 26 | −6 bp | −6 bp | −1 bp | −4 bp | −5 bp | −5 bp |
| 27 | −6 bp | −6 bp | −1 bp | −4 bp | −5 bp | −5 bp |
| 28 | −6 bp | −9 bp | −1 bp | −4 bp | −2 bp | −5 bp |

TABLE 14
ddPCR FLC drop-off percentages for
immature embryos from TMTA0609.

| Shot | Rupture Disk | Shooting | ddPCR |
|---|---|---|---|
| Number | Pressure (psi) | Distance | drop-off (%) |
| 1 | 650 | L2 (2 cm) | 0.91 |
| 2 | 650 | L2 (2 cm) | 3.63 |
| 3 | 650 | L2 (2 cm) | 6.87 |
| 4 | 650 | L2 (2 cm) | 4.10 |

TABLE 15
Summary of FLC ddPCR results obtained
for pooled leaf samples from TMTA0609.

| ddPCR drop−off % | Shot 2 | | Shot 3 | | Shot 4 | |
|---|---|---|---|---|---|---|
| category | # pools | % pools | # pools | % pools | # pools | % pools |
| ≤10% | 45 | 76.3 | 16 | 43.3 | 44 | 73.3 |
| 10-≤25% | 13 | 22.0 | 10 | 27.0 | 6 | 10.0 |
| 25-≤50% | 1 | 1.7 | 5 | 13.5 | 9 | 15.0 |
| >50% | 0 | 0 | 6 | 16.2 | 1 | 1.7 |
| Total | 59 | 100 | 37 | 100 | 60 | 100 |

TABLE 16
Summary of FLC ddPCR results obtained for individual shoots from TMTA0609.

| | | | | % Drop−off | | | | | | | |
|---|---|---|---|---|---|---|---|---|---|---|---|
| | | | # of individual | 0% | 17% | 33% | 50% | 67% | 83% | 100% | Non |
| Pool reference | Shot | Pool % drop−off | plants sampled | (WT) | (1 allele) | (2 alleles) | (3 alleles) | (4 alleles} | (5 alleles) | (6 alleles) | assigned |
| [symbol] | 2 | 11 | 24 | 18 | 5 | 0 | 3 | 0 | 0 | 0 | 0 |
| [symbol] | 2 | 19 | 21 | 11 | 5 | 0 | 5 | 0 | 0 | 0 | 0 |
| [symbol] | 2 | 23 | 19 | 6 | 1 | 1 | 9 | 2 | 0 | 0 | 0 |
| [symbol] | 2 | 14 | 21 | 11 | 9 | 1 | 0 | 0 | 0 | 0 | 0 |
| [symbol] | 2 | 20 | 18 | 10 | 1 | 0 | 3 | 0 | 0 | 0 | 4 |
| [symbol] | 2 | 21 | 20 | 12 | 3 | 1 | 0 | 3 | 0 | 0 | 1 |
| [symbol] | 2 | 21 | 18 | 7 | 2 | 4 | 2 | 3 | 0 | 0 | 0 |
| [symbol] | 2 | 27 | 16 | 4 | 5 | 4 | 2 | 0 | 1 | 0 | 0 |
| [symbol] | 2 | 23 | 20 | 11 | 1 | 1 | 4 | 0 | 1 | 2 | 0 |
| [symbol] | 2 | 10 | 17 | 11 | 0 | 6 | 0 | 0 | 0 | 0 | 0 |
| [symbol] | 3 | 30 | 14 | 1 | 0 | 13 | 0 | 0 | 0 | 0 | 0 |
| [symbol] | 3 | 13 | 24 | [symbol] | 4 | 0 | 0 | 0 | 3 | 0 | [symbol] |
| [symbol] | 3 | 15 | 22 | 18 | 0 | 0 | 0 | 0 | 1 | 2 | 1 |
| [symbol] | 3 | 49 | 16 | 5 | [symbol] | 0 | 0 | 6 | 1 | 2 | 2 |
| [symbol] | 3 | 58 | 15 | 4 | 0 | 0 | 2 | 3 | 1 | 4 | 1 |
| [symbol] | 3 | 71 | 15 | 0 | [symbol] | 1 | 3 | 0 | 9 | 2 | 0 |
| [symbol] | 3 | 21 | 19 | 9 | 0 | 0 | 10 | 0 | [symbol] | [symbol] | [symbol] |
| [symbol] | 3 | 57 | 13 | 8 | 2 | 0 | 0 | 1 | 1 | 8 | 0 |
| [symbol] | 3 | 54 | 15 | 0 | 0 | 1 | [symbol] | 2 | 0 | 5 | 0 |
| [symbol] | 3 | 58 | 15 | 0 | $ | 1 | 1 | 3 | 1 | 5 | 0 |
| [symbol] | 3 | 24 | 19 | 9 | 2 | 3 | 3 | 1 | 0 | 0 | 1 |
| [symbol] | 3 | 48 | 15 | 2 | 1 | 1 | 3 | 2 | 3 | 3 | 0 |
| [symbol] | 3 | 37 | 18 | 5 | 0 | 8 | 2 | 0 | 4 | 0 | [symbol] |
| [symbol] | 3 | 80 | 15 | 0 | 1 | 4 | 1 | [symbol] | 2 | 2 | 0 |
| [symbol] | 3 | 24 | 20 | 10 | 0 | 4 | [symbol] | 2 | 2 | 0 | 1 |
| [symbol] | 3 | 45 | 15 | 4 | 3 | 1 | 0 | 0 | 7 | 0 | 0 |
| [symbol] | 4 | 12 | 20 | 9 | 8 | 2 | 1 | 0 | 0 | 0 | 0 |
| [symbol] | 4 | 30 | 17 | 6 | 5 | 3 | 2 | [symbol] | [symbol] | 0 | 0 |
| [symbol] | 4 | 13 | 24 | 19 | 1 | 2 | 2 | 0 | 0 | 0 | [symbol] |
| [symbol] | 4 | 24 | 20 | 7 | 2 | 2 | 4 | 5 | [symbol] | 0 | 0 |
| [symbol] | 4 | 45 | 15 | 3 | 4 | 0 | 2 | 6 | 0 | 0 | [symbol] |
| [symbol] | 4 | 48 | 15 | [symbol] | 1 | 0 | 1 | 2 | 1 | 4 | 2 |
| [symbol] | 4 | 67 | 15 | 4 | 0 | 1 | 3 | 0 | 3 | 3 | 1 |
| [symbol] | 4 | 48 | 15 | 6 | [symbol] | 2 | [symbol] | 2 | [symbol] | 4 | 0 |

[symbol]

indicates data missing or illegible when filed

### FIGURES

FIG. 1: Multiple sequence alignment depicting the position of the Cas9 gRNA in exon 4 of the three wheat mlo homoeologues. The PAM site is shown boxed, the gRNA recognition site is underlined. Single nucleotide polymorphisms are shown in bold.

FIG. 2: Schematic drawing of the ddPCR design indicating the location, orientation and sequences of the different probes and primers. The position of the predicted cut site by Cas9 and the R158Q edit are also indicated. The edit probe only binds to alleles modified by HDR, whereas the drop-off probe loses its binding site when insertions or deletions are created by NHEJ.

FIG. 3: Validation of the ddPCR assay by NGS analysis

FIG. 4: ddPCR assay sensitivity established by serial dilution of HDR and NHEJ synthetic templates in a constant background (200 ng) of WT genomic DNA. The limit of detection was ˜0.1% for NHEJ and ˜0.04% for HDR, as determined by comparison with WT genomic DNA controls. Data are means±standard deviation of two merged wells per dilution and three merged wells for the WT control.

FIG. 5: Plate of RNP bombarded immature wheat embryos Zone 1 (center target area), Zone 2 (inner circle target area) and Zone 3 (outer circle target area).

FIG. 6: Process for leaf sampling of pools from PlantCon™ containers: Shoots regenerated from immature embryo derived callus (A), open-container showing shoots with long leaves (B), container with shoots pre-trimmed prior to sampling (C), container with shoots from which uniform leaf samples have been sampled (D), dish with sampled pooled leaf explants (E) and container ready for shoot regrowth and 6 ml sample tube (F).

FIG. 7: Overview of the complete 3-step selection system used to identify RNP-induced indels in wheat plants.

FIG. 8: A detailed scheme of the selection/analysis process used to identify Cas9 RNP-induced indels in wheat plants, indicating timelines and processes from embryo RNP bombardment to transfer of structured embryogenic callus tissue to PlantCon™ containers. Time point for sampling 1 is indicated in bold.

FIG. 9: A detailed scheme of the selection/analysis process used to identify Cas9 RNP-induced indels in wheat plants, indicating timelines and processes from transfer of structured embryogenic callus tissue to PlantCon™ containers to transfer of mutant plants to the greenhouse. Time points for sampling 2 and sampling 3 are indicated in bold.

FIG. 10: Two-dimensional plots generated from Quantasoft Software for mlo ddPCR analysis preformed on immature embryo samples TMTA0233-B01$001 ddPCR drop-off 5.6% (A), TMTA0233-B02$001 ddPCR drop-off 4.0% (B) and TMTA0233-B05$001 ddPCR drop-off 5.0% (C). NHEJ (FAM+, HEX−)=upper left quadrant, WT alleles (FAM+, HEX+)=upper right quadrant and negative droplets (FAM−, HEX−)=lower left quadrant.

FIG. 11: Bar chart showing the ddPCR mlo drop-off percentage categories obtained from pools of analyzed shoots from the 3 shots of TMTA0233 (categories <10% drop-off, 10-25% drop-off, 25-50% drop-off and >50% drop-off).

FIG. 12: Two-dimensional plots generated from Quantasoft Software for representative pools showing ddPCR mlo drop-off percentages, <10% TMTA0233-045-B03$001 (A), 10-25% TMTA0233-058-B02$001 (B), 25-50% TMTA0233-040-B02$001 (C) and >50% TMTA0233-035-B03$001 (D). NHEJ (FAM+, HEX−)=upper left quadrant, WT alleles (FAM+, HEX+)=upper right quadrant and negative droplets (FAM−, HEX−)=lower left quadrant.

FIG. 13: Two-dimensional plots generated from Quantasoft Software for representative individual shoots showing ddPCR mlo drop-off percentages of 0% (A), 50% (B) and 100% (C). NHEJ (FAM+, HEX−)=upper left quadrant, WT alleles (FAM+, HEX+)=upper right quadrant and negative droplets (FAM−, HEX−)=lower left quadrant.

FIG. 14: PCR size-fragment analysis of 86 individual plants (primer combination Seq ID NO: 1/Seq ID NO: 2). 6 plants from pool TMTA0233-035-B04, 25 plants from pool TMTA0233-037-B01, 14 plants from pool TMTA0233-037-B02, 16 plants from pool TMTA0233-044-B03 and 25 plants from pool TMTA0233-046-B02 were analyzed. Plants marked with arrows showed 0% drop-off during ddPCR analysis.

FIG. 15: Multiple sequence alignment depicting the position of the Cas12a gRNA in exon 4 of the three wheat mlo homoeologues. The PAM site is shown boxed, the gRNA recognition site is underlined. Single nucleotide polymorphisms are shown in bold.

FIG. 16: Schematic drawing of the ddPCR design indicating the location, orientation and sequences of the different probes and primers. The position of the predicted cut site by Cas 12a and the R158Q edit are also indicated. The edit probe only binds to alleles modified by HDR, whereas the drop-off probe loses its binding site when insertions or deletions are created by NHEJ.

FIG. 17: A detailed scheme of the selection/analysis process used to identify Cas 12a RNP-induced indels in wheat plants, indicating timelines and processes from embryo RNP bombardment to transfer of structured embryogenic callus tissue to PlantCon™ containers. Time point for sampling 1 is indicated in bold.

FIG. 18: A detailed scheme of the selection/analysis process used to identify Cas 12a RNP-induced indels in wheat plants, indicating timelines and processes from transfer of structured embryogenic callus tissue to PlantCon™ containers to transfer of mutant plants to the greenhouse. Time points for sampling 2 and sampling 3 are indicated in bold.

FIG. 19: Two-dimensional plots generated from Quantasoft Software for mlo ddPCR analysis preformed on immature embryo samples TMTA0527-B01$001 ddPCR drop-off 1.9% (A), TMTA0527-B02$001 ddPCR drop-off 2.7% (B) and TMTA0527-B05$001 ddPCR drop-off 1.6% (C). NHEJ (FAM+, HEX−)=upper left quadrant, WT alleles (FAM+, HEX+)=upper right quadrant and negative droplets (FAM−, HEX−)=lower left quadrant.

FIG. 20: Two-dimensional plots generated from Quantasoft Software for representative pools showing ddPCR mlo drop-off percentages, <10% TMTA0527-031-B01$001 (A), 10-25% TMTA0527-033-B02$001 (B), 25-50% TMTA0527-039-B04$001 (C) and >50% TMTA0527-044-B06$001 (D). NHEJ (FAM+, HEX−)=upper left quadrant, WT alleles (FAM+, HEX+)=upper right quadrant and negative droplets (FAM−, HEX−)=lower left quadrant.

FIG. 21: Two-dimensional plots generated from Quantasoft Software for representative individual shoots showing ddPCR mlo drop-off percentages of 0% (A), 50% (B) and 100% (C). NHEJ (FAM+, HEX−)=upper left quadrant, WT alleles (FAM+, HEX+)=upper right quadrant and negative droplets (FAM−, HEX−)=lower left quadrant.

FIG. 22: PCR size-fragment analysis of individual plants recovered from different pools of TMTA0527 (primer combination Seq ID NO: 1/Seq ID NO: 2).

FIG. 23: PCR size-fragment analysis of 62 individual plants (primer combination Seq ID NO: 1/Seq ID NO: 2) regenerated from a single embryo explant (TMTA0527-045). 18 plants from pool TMTA0527-045-B02, 13 plants from pool TMTA0527-045-B03, 16 plants from pool TMTA0527-045-B04 and TMTA0527-045-B05 were analyzed. Plants selected for NGS are indicated by arrows (previously characterized by as 0%, 50% or 100% ddPCR drop-off for the A sub-genomic allele of mlo).

FIG. 24: Sequence alignment depicting the position of the Cas12a gRNA in the 3 subgenomic copies of TaAGL33 (FLC). The PAM site is shown boxed, and the gRNA recognition site is underlined. Single nucleotide polymorphisms are shown in bold.

FIG. 25: Schematic drawing of the FLC ddPCR design indicating the location, orientation and sequences of the different probes and primers to detect indels generated at the Cas 12a (cr-FLC-G1) cut site.

FIG. 26: Distribution of RNP-targeted FLC alleles in individual plants recovered from 3 independent shots from experiment TMTA0609.

FIG. 27: Sequences

### EXAMPLES

### Chemicals and Common Methods

Unless indicated otherwise, cloning procedures carried out for the purposes of the present invention including restriction digest, agarose gel electrophoresis, purification of nucleic acids, Ligation of nucleic acids, transformation, selection and cultivation of bacterial cells were performed as described (Sambrook et al., 1989). Sequence analyses of recombinant DNA were performed with a laser fluorescence DNA sequencer (Applied Biosystems, Foster City, CA, USA) using the Sanger technology (Sanger et al., 1977). Unless described otherwise, chemicals and reagents were obtained from Sigma Aldrich (Sigma Aldrich, St. Louis, USA), from Promega (Madison, WI, USA), Duchefa (Haarlem, The Netherlands) or Invitrogen (Carlsbad, CA, USA). Restriction endonucleases were from New England Biolabs (Ipswich, MA, USA) or Roche Diagnostics GmbH (Penzberg, Germany). Oligonucleotides were synthesized by Eurofins Eurofins Genomics (Ebersberg, Germany) or Integrated DNA Technologies (Coralville, IA, USA).

### Example 1-Cas9 RNP Targeting of mlo

gRNA Design for Cas9 Targeting of mlo

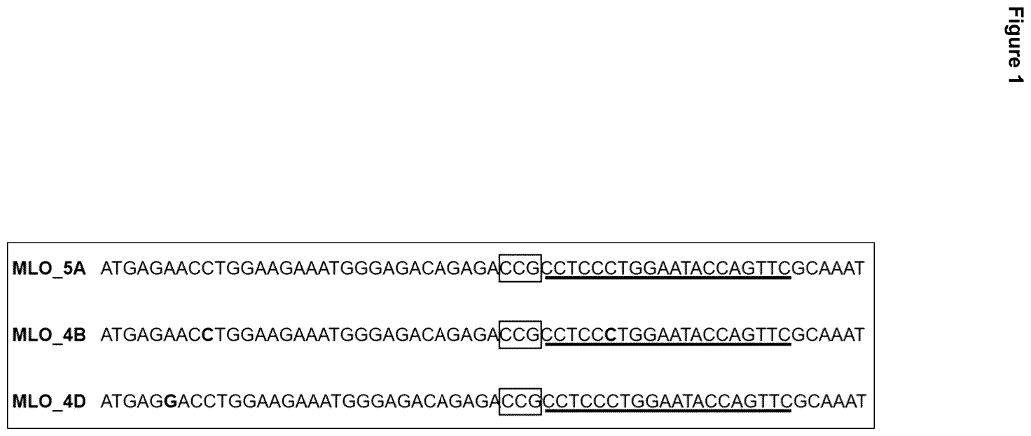

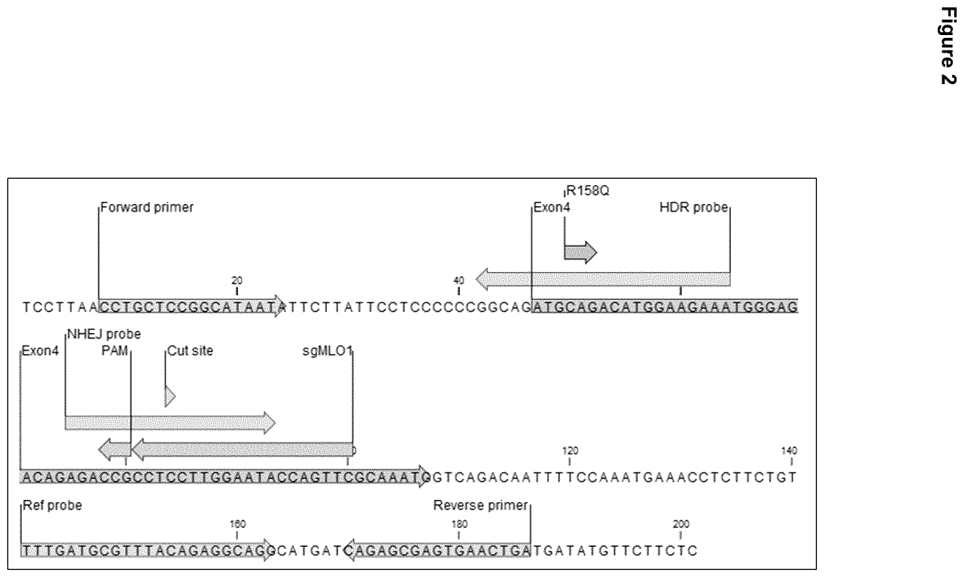

Building upon previous work (Gil-Humanes et al. 2017) we designed a synthetic crRNA composed of a 16 bp direct repeat sequence and a 20 bp protospacer targeting the wheat mildew resistance locus O (mlo). The mlo gene encodes a seven-transmembrane domain protein involved in resistance to the fungal pathogen Blumeria graminis. The recognition site of the crR-NA is located on the antisense strand within exon 4 of mlo [5′-GAACTGGTATTCCAAGGAGG (CGG)-3]′, with PAM site between brackets. The designed guide is specific for the 5A and 4D alleles of mlo and shows one mismatch with the 4B allele at position 6 from the PAM sequence (FIG. 1).

Design of a Droplet Digital PCR (ddPCR) Assay to Simultaneously Identify Cas9 RNP-Induced mlo Indel Mutations and Precise Edits

Current methods to detect genome editing events include gel-based systems, artificial reporter assays, high resolution melting curve analysis and next-generation sequencing. Droplet digital PCR is a rapid alternative to these methods enabling rapid and systematic quantification of genome editing outcomes at endogenous loci. In a droplet digital PCR system, each PCR sample is partitioned into many droplets. PCR amplification occurs simultaneously in each droplet. At the end of the run, each droplet is individually assessed for the presence (positive) or absence (negative) of a fluorescent signal. Using a Poisson statistical analysis, the ratio of positive to negative droplets yields absolute quantification of the initial number of copies of the target sequence.

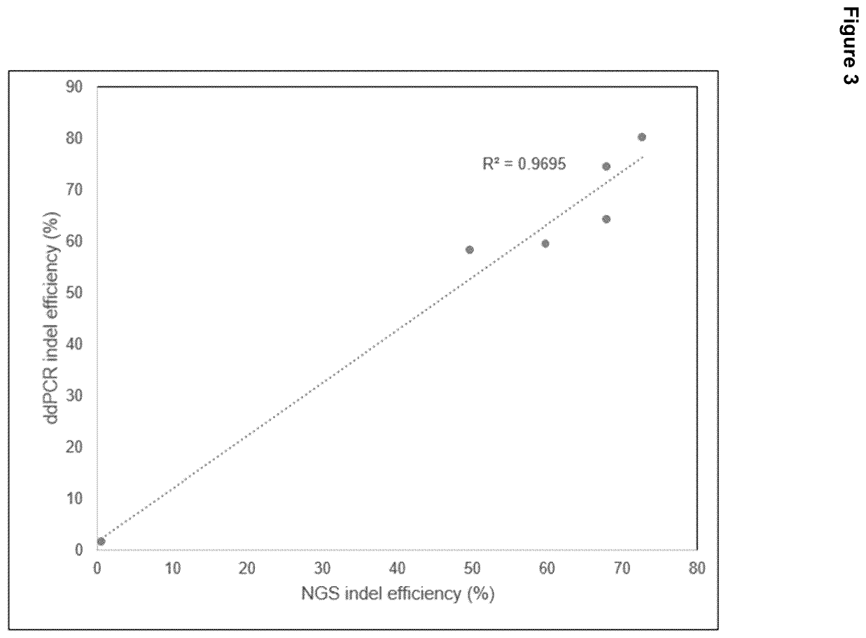

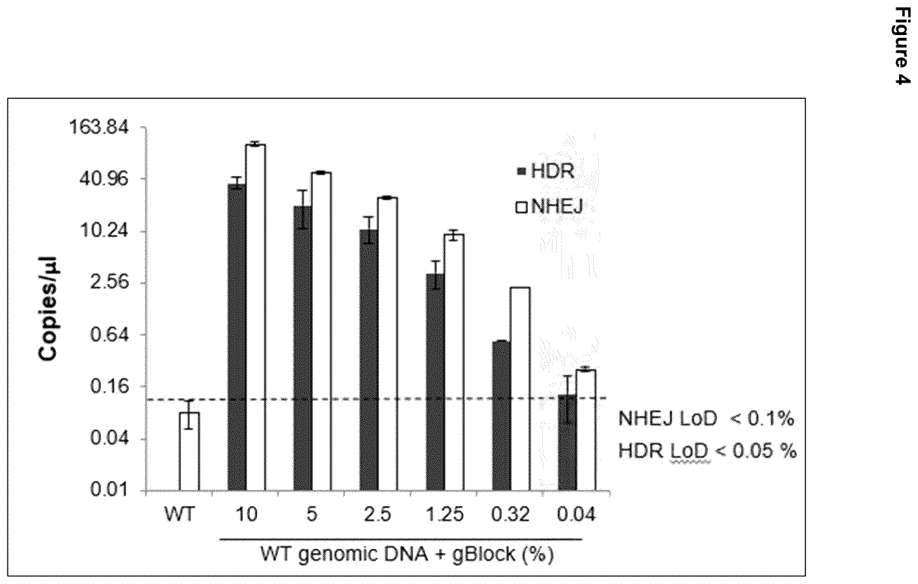

Although the current application is focused towards detecting insertions and deletions, a ddPCR assay was designed capable of simultaneously measuring NHEJ and HDR at endogenous loci. To this end, we designed three kinds of probes, all located within one amplicon (FIG. 2). The first, a reference probe, is labeled with FAM and located away from the mutagenesis site. This probe counts all genomic copies of the target. The second, a so-called drop-off probe, is labeled with HEX and is located where the Cas9 nuclease cuts the mlo target. If Cas9 induces NHEJ, the drop-off probe loses its binding site, resulting in loss of HEX and leaving only the FAM signal of the reference probe. The third probe, also FAM-labeled, binds to the desired DNA edit, causing a gain of additional FAM signal when precise edits are introduced. With this assay, indel mutations, WT alleles and precise edits can be detected as distinct, clearly separated droplets with high sensitivity and low background signal.

ddPCR assays were designed against the mlo 5A allele using Primer3Plus software with modified settings compatible with the master mix: that is, 50 mM monovalent cations, 3.0 mM divalent cations, and 0 mM dNTPs with SantaLucia 1998 thermodynamic and salt correction parameters. The predicted nuclease cut site (3 bp from the PAM) was positioned mid-amplicon, with 70-100 bp flanking sequence either side up to the primer binding sites. To avoid loss of binding sites, primers and reference probe were designed away from the cut site. In addition, a dark, 3′-phosphorylated non-extendible oligonucleotide was designed to prevent the edit probe from binding to the WT sequence.

PCR primers were designed according to the following guidelines: primer length of 17-24 bases, primer melting temperature of 55 to 60° C. with an ideal temperature of 58° C., melting temperatures of the two primers differ by no more than 2° C., primer GC content of 35-65%, amplicon size of 100-250 bases.

Considerations for probe design were as follows: probes can bind to either strand of the target, probe GC content of 35-65%, no G at the 5′ end to prevent quenching of the 5′ fluorophore, melting temperature of the drop-off probe ranges from 61° C. to 64° C. with an ideal temperature of 62° C., length of the drop-off probe is less than 20 bases, melting temperatures of the reference and edit probe range from 63° C. to 67° C. with an ideal temperature of 65° C., length of the reference and edit probe of 20-24 bases. Preferably, probes should have a Tm 4-8° C. higher than the primers. Primer and probe designs were also screened for complementarity and secondary structure with the maximum ΔG value of any self-dimers, hairpins, and heterodimers set to −9.0 kcal/mole. All primers and probes were designed against the 5A allele of the wheat mlo gene.

The optimal annealing temperature was empirically determined using a temperature gradient PCR.

Synthetic dsDNA fragments (gBlocks, Integrated DNA Technologies) were used as positive controls for assay validation, HDR-positive controls contain the R158Q substitution at the desired edit site, whereas NHEJ-specific controls have a 1-bp insert at the predicted nuclease cut site. Lyophilized gBlocks were resuspended in 300 μl of TE and stored at min 20° C. Three additional dilutions in TE resulted in a master stock of approximately 600 copies/μl that was confirmed by ddPCR quantification. High-copy gBlock stocks were kept in a post-PCR environment to avoid contamination.

20× ddPCR mixes were composed of 18 μM forward (Seq ID NO: 1) and 18 μM reverse (Seq ID NO: 2) primers, 5 μM reference probe (Seq ID NO: 3), 5 μM edit probe (Seq ID NO: 4), 5 μM drop-off probe (Seq ID NO: 5), and 10 μM dark probe (Seq ID NO: 6). The following reagents were mixed in a 96-well plate to make a 25-μl reaction: 11 μl of ddPCR Supermix for Probes (no dUTP), 1.1 μl of 10× assay mix (BioRad Laboratories), 10U of HindIII-HF, 100-250 ng of genomic DNA in water, and water up to 22 μl.

Droplets were generated using a QX100 Droplet Generator according to the manufacturer's instructions (Bio-Rad Laboratories) and transferred to a 96-well plate for standard PCR on a C1000 Thermal cycler with a deep well block (BioRad Laboratories).

Thermal cycling consisted of a 10 min activation period at 95° C. followed by 40 cycles of a two-step thermal profile of 30 s at 95° C. denaturation and 3 min at 60° C. for combined annealing-extension and 1 cycle of 98° C. for 10 min.

After PCR, the droplets were analyzed using a QX100 Droplet Reader (BioRad Laboratories) in ‘absolute quantification’ mode. To enable proper gating for precise edits and indel events, experiments were performed using both negative and positive controls (non-modified genomic DNA and gBlocks containing the R158Q mutation, respectively). In two-dimensional plots, droplets without templates were gated as negative population. Droplets containing only NHEJ (FAM+, HEX−), only HDR alleles (FAM++, HEX−) or only WT alleles (FAM+, HEX+) were manually gated as separate populations. Allelic frequencies were quantified using the QuantaSoft v. 1.2.10.0 software (BioRad Laboratories).

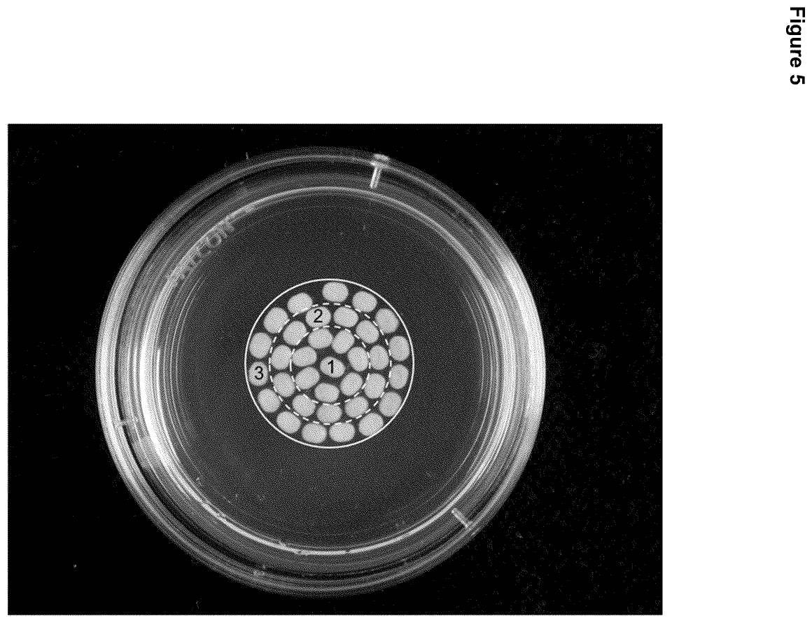

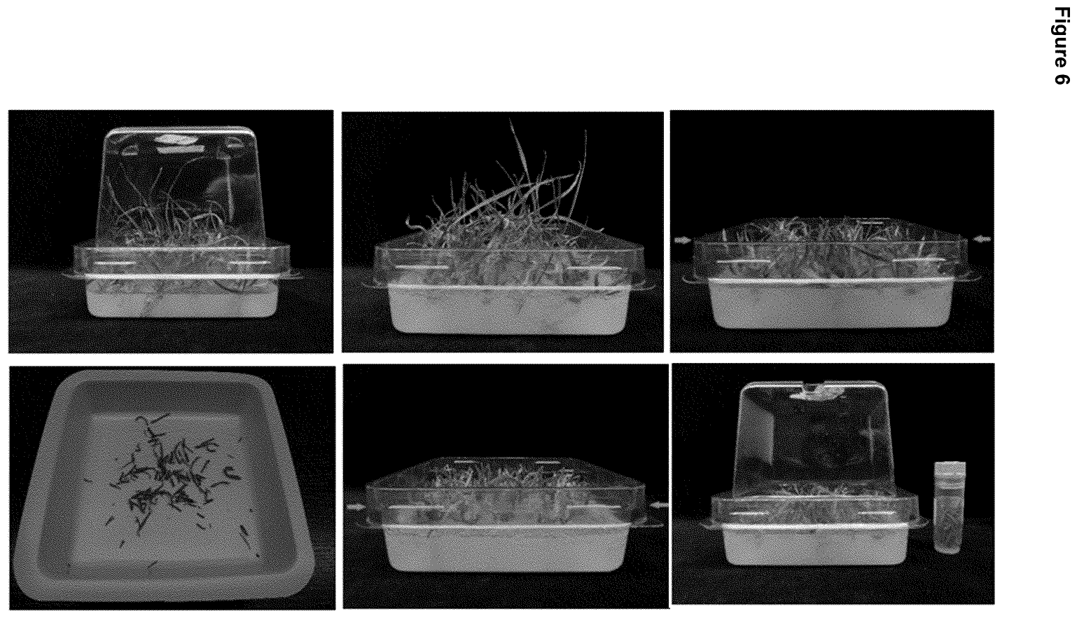

The designed ddPCR assay was verified by next-generation sequencing (NGS) of the target region using a pair of primers specific for the A sub-genome allele of the wheat mlo gene (Seq ID NO: 7/Seq ID NO: 8). The amplicons were purified and subjected to deep-sequencing (2×250 bp paired ends) (GENEWIZ, Germany, GmbH) using an Illumina MiSeq System. A very good correlation (R2=0.96) was observed between the indel allele frequencies detected by ddPCR and NGS across different samples, demonstrating the sensitivity and reliability of the ddPCR assay (FIG. 3).

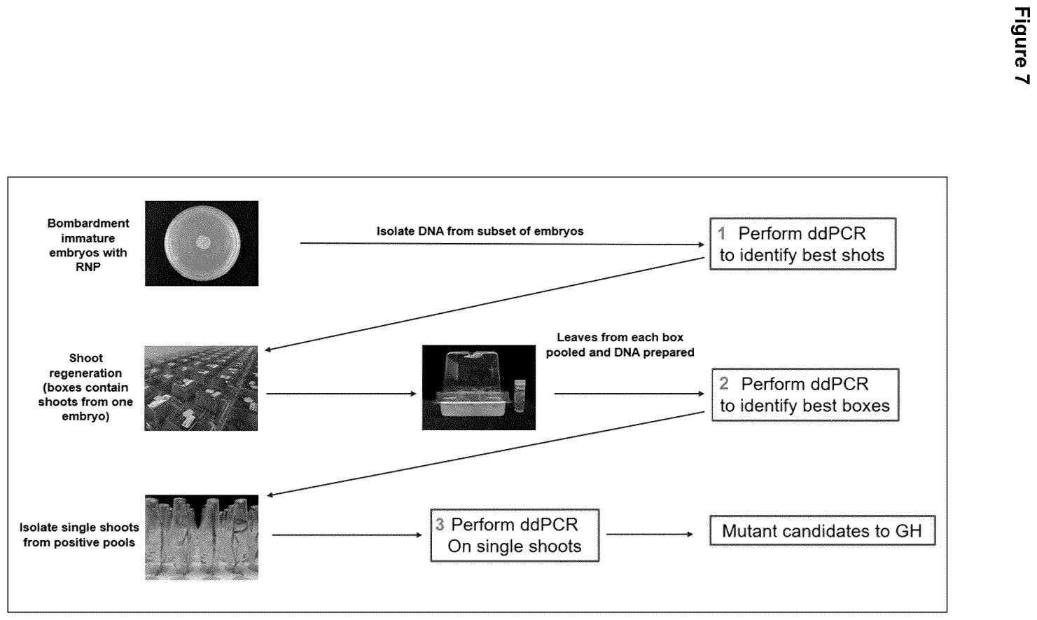

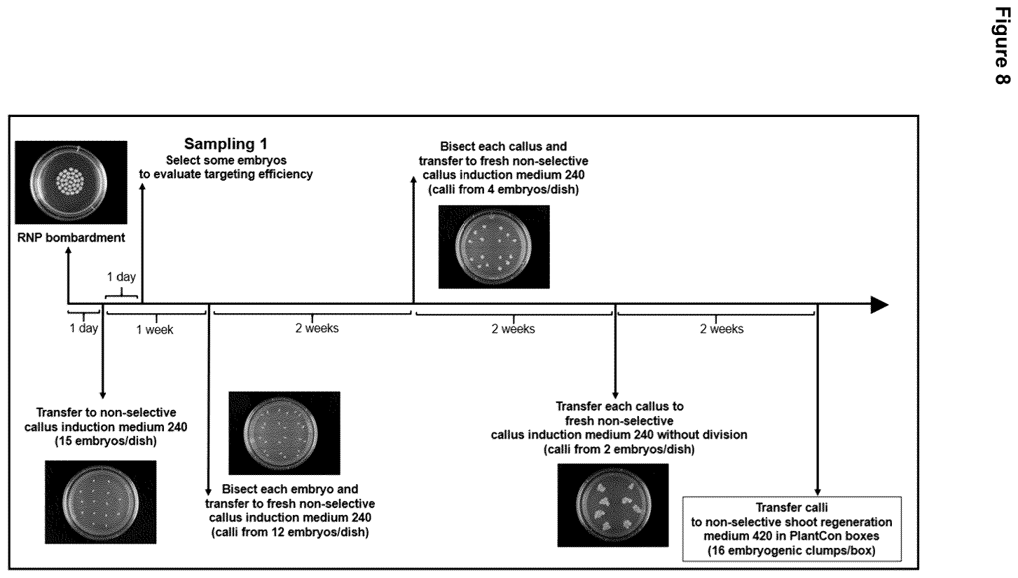

To calculate the ddPCR assay's limit of detection, we spiked wild-type genomic wheat DNA with different amounts of the HDR- and NHEJ-specific gBlocks (Seq ID NO: 9/Seq ID NO: 10) and found that the assay was reproducible and linear over a wide range of input DNA (FIG. 4). The limit of detection was approximately 0.1% for NHEJ. and well below 0.04 for % HDR alleles. This indicates that at least one indel or precise edit event from 1,000 copies of the genome can be captured by the assay.

### Wheat Donor Plant Growth for Immature Embryo Isolation

Donor plants of cv. Fielder were grown under controlled-environment conditions to ensure optimal immature embryo quality. Typical growth conditions were day/night temperatures of 20° C.+/−1° C. and 18° C.+/−1° C., 65% relative humidity with a 16 h photoperiod (400 μmol/m²/s illumination at table level) provided by a mixture of 600W high pressure sodium lamps and 400W metal halide lamps.

### Preparation of Wheat Immature Embryos for Bombardment

Immature seeds were harvested from donor plants containing embryos around 2 mm in length, peeled and sterilized in 70% v/v ethanol for 1 min and followed by 10 min in 10% v/v sodium hypochlorite (ACROS bleach containing 10-15% active chlorine). Finally, the immature seeds were washed several times with demineralized water.

Embryos were aseptically excised from the immature seeds using a binocular microscope (Model MZ6, Leica) taking care to slice-off the embryo axis through the green seed coat during preparation. Subsequent steps for embryo preparation were performed using a modified procedure essentially as described by Ishida et al. (2015). Immature embryo explants were transferred to 3.5 cm Petri dishes (Falcon 351008) containing 4.5 ml of non-selective callus induction medium WLS, herewith designated callus induction medium 240. Embryos were arranged in a central circle of 1.5 cm (25-50 embryos/dish with the scutellum side facing upwards).

### Preparation of Cas9 RNP Complexes

Purified Cas9 nuclease, universal 67mer tracerRNA and mlo-specific crRNA were ordered from IDT (Integrated DNA Technologies) for RNP assembly.
crRNA: custom and user-defined crRNA that binds to 20 bases on the DNA strand that is opposite to the NGG, PAM sequence
tracrRNA: universal 67mer transactivating crRNA (tracrRNA) that contains proprietary chemical modifications conferring increased nuclease resistance. It hybridizes to crRNA to activate the Cas9 enzyme
Cas9 nuclease (Alt-R® S.p. Cas9 Nuclease 3NLS): recombinant S. pyogenes Cas9 nuclease, purified from E. coli strain expressing codon optimized Cas9. Contains 1 N-terminal nuclear localization sequence (NLS), 2 C-terminal NLS and a C-terminal 6-His tag.

For RNP complex assembly, mlo-specific crRNA and tracrRNA were mixed in equimolar amounts to create a final duplex concentration of approximately 4 μM. The RNA complex was heated to 95° C. for 5 min in a heating-block, cooled to room temperature and mixed in equimolar concentration with Cas9 nuclease in Cas9 Reaction Buffer (10× stock=200 mM HEPES, 100 mM MgCl26H2O, 5 mM DTT, 1500 mM KCl prepared in RNase-free water, pH 7.5). The mixture was incubated at room temperature for 10 min and then transferred to ice.

Biolistic Delivery of Cas9 RNP Complexes into Wheat

RNP delivery into immature embryos was conducted using the Biolistic®PDS-1000/He Particle Delivery System (Bio-Rad) essentially as described by Liang et al. (2018). RNP complexes were mixed with 0.6 μm gold particles (BioRad) and 15 μl aliquots were spread over the central region of each macrocarrier and airdried for 30 min in a laminar flow bench prior to delivery. For each shot 200 μg of gold particles and 2 μg of Cas9 protein complexed with crRNA were delivered.

### Culture of Bombarded Wheat Immature Embryos and Plant Regeneration

Following bombardment, the immature embryos were incubated on the same plates for 24 h in the dark (25° C.+/−1° C., 55% relative humidity in an MLR-352H-PE Panasonic incubator) and then transferred to fresh non-selective callus induction medium 240 (9 cm dishes containing 35-40 ml of medium with up to 15 embryos/dish).

Eight days after bombardment immature embryos were longitudinal bisected into 2 pieces under a binocular microscope and transferred to fresh non-selective callus induction medium 240 (9 cm dishes containing 35-40 ml of medium, 12 embryos/dish) and cultured in the dark under the same conditions.

After 2 weeks, the bisected immature embryos were once more bisected under a binocular microscope and transferred to fresh non-selective callus induction medium 240 (9 cm dishes containing 35-40 ml of medium, 4-6 embryos/dish) and cultured in the dark under the same conditions. After a further 2 weeks, the calli from each embryo were transferred intact to fresh nonselective callus induction medium 240 (9 cm dishes containing 35-40 ml of medium, 2 embryos/dish) and cultured in the dark under the same conditions.

Two weeks later, small pieces of structured embryogenic callus tissue were transferred to a non-selective regeneration medium, LSZ (Ishida et al., 2015), herewith designated regeneration medium 420. Regeneration medium 420 was prepared in PlantCon™™ containers (MP Biomedicals, Catalogue Nr. 26-722-06), 100 ml medium/container. The embryogenic calli arising from one embryo were transferred to one PlantCon™ container (max. 16 pieces/container). Where more than 16 embryogenic calli were recovered from one embryo, additional regeneration containers were utilized. PlantCon™ containers were incubated in the light for approximately 6 weeks (23° C.+/−1° C., 16 h photoperiod).

Shoots from PlantCon™ containers were transferred individually to De Wit tubes (Duchefa Biochemie) containing 10 ml of non-selective rooting medium WRM, essentially a modification of medium R (Sparks and Jones, 2009), solidified with 0.15% w/v Gelrite (Duchefa Biochemie) and cultured in the light (23° C.+/−1° C., 16 h photoperiod). Rooted plants were transferred to the greenhouse.

### Tissue Sampling and DNA Isolation

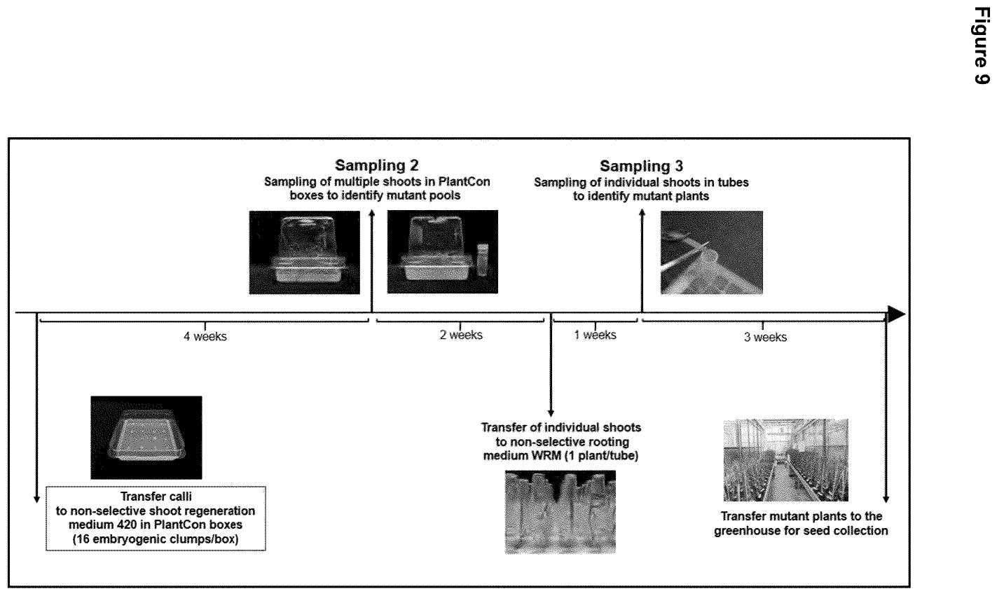

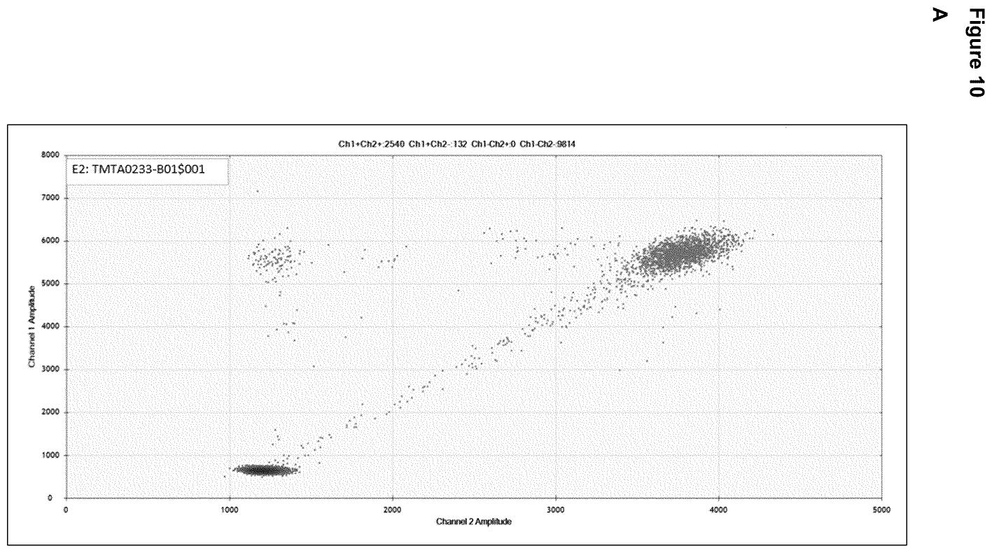

Sampling 1-Two days after bombardment, 5 immature embryos were randomly selected from Zone 2 (FIG. 5) of each bombarded plate. The embryos were bulked as one sample, collected in 2 ml tubes (Eppendorf® Safe-Lock) and stored at −80°. Samples were ground in a Retsch Mixer Mill MM300/400 for 60 sec. Genomic DNA extraction was performed using the Qiagen Dneasy Plant Mini Kit (Catalogue Nr. 69106) according to the manufacturer's instructions. Final DNA concentrations were measured, and plates stored at 4° C. until use for downstream analysis.

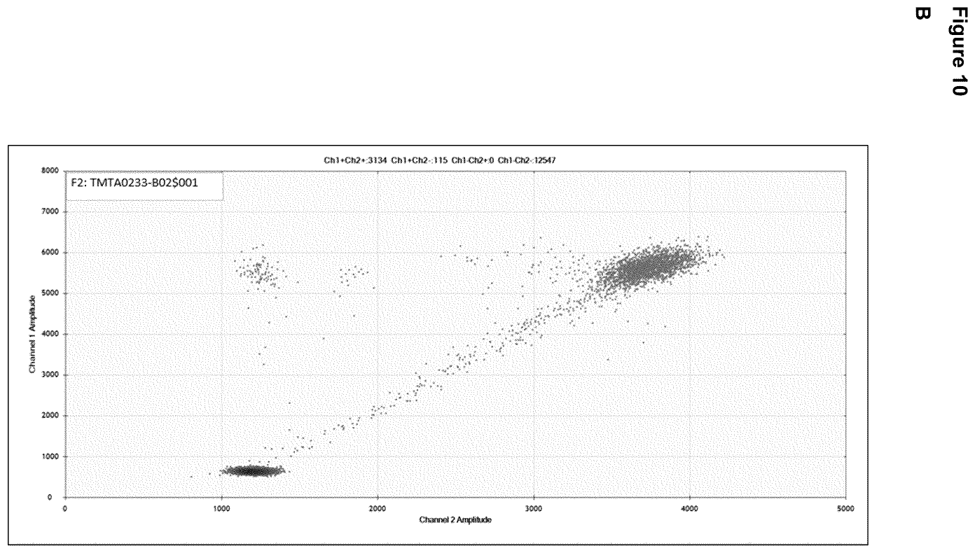

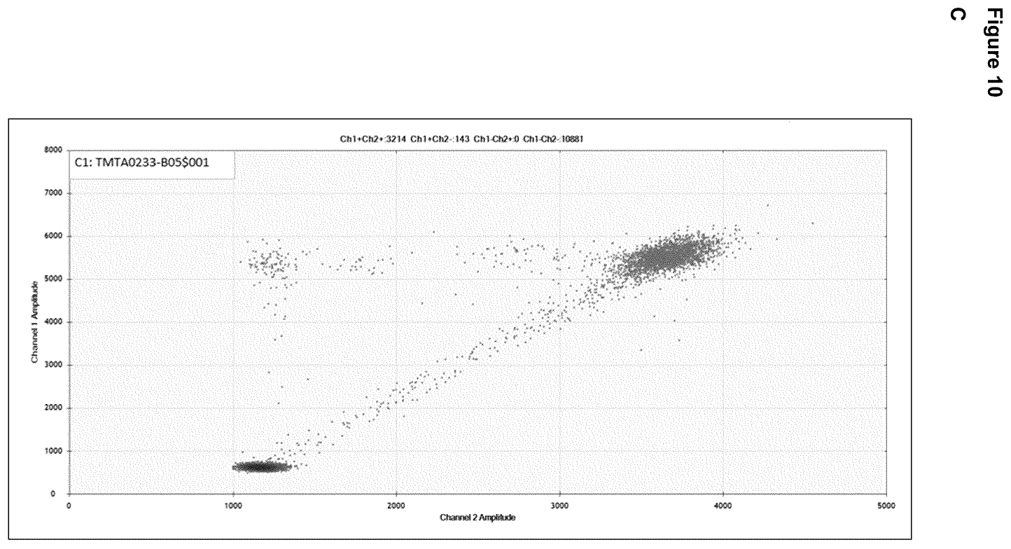

Sampling 2-Following 4 weeks of culture of structured embryogenic callus on non-selective regeneration medium 420 in PlantCon™ containers, leaf pieces were harvested from regenerating shoots. Care was taken to ensure that similar sized leaf pieces were taken from all regenerating shoots, this was best achieved by first cutting off all longer leaf tips with sterile scissors and then cutting leaf pieces of approximately 5 mm in length from the remaining tissues (FIG. 6). Leaf pieces originating from each PlantCon™ container were pooled and transferred to 6 ml screw-capped tubes (Micronic MP 32301) and stored at −80°. Samples were freeze dried overnight and genomic DNA extracted using a procedure based on the LGC GENOMICS sbeadex™ Maxi Plant Kit with KingFisher automation. Final DNA concentrations were measured, and plates stored at 4° C. until use for downstream analysis.

Sampling 3-Following 1-2 weeks of culture of individual shoots in De Wit tubes containing 10 ml of WRM medium, leaf pieces were harvested from shoots. Samples were collected in 1.4 ml push-cap tubes in a 96-sample carrier rack (Micronic MP 226RP) and stored at −80°. Genomic DNA was extracted using a procedure based on the LGC GENOMICS sbeadex™ Maxi Plant Kit with KingFisher automation. Final DNA concentrations were measured, and plates stored at 4° C. until use for downstream analysis.

### Selection/Analysis Process to Identify Cas9 RNP Indels in mlo

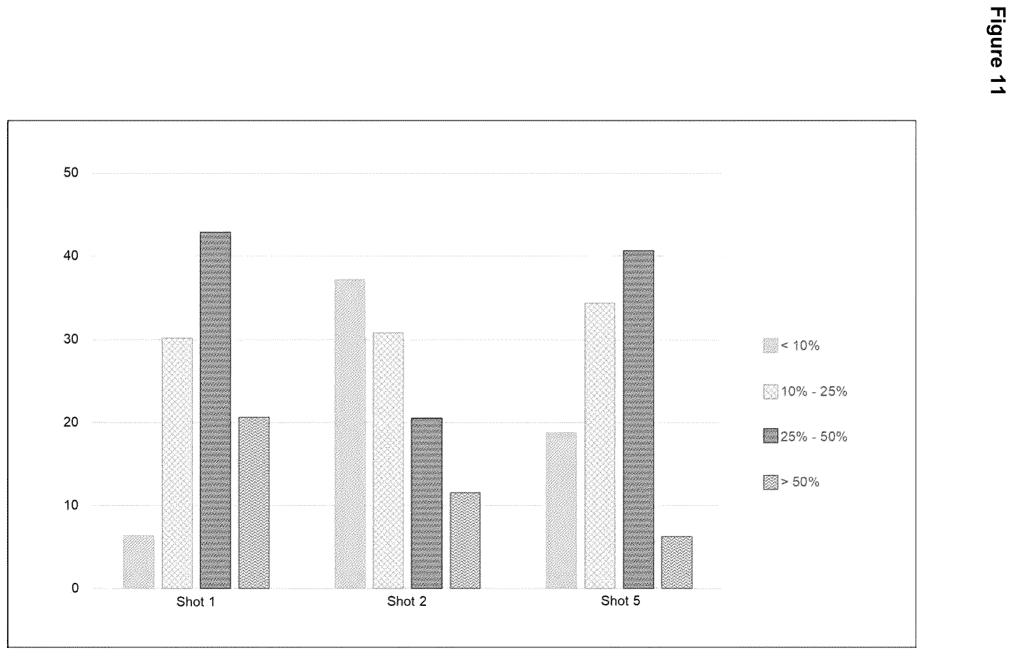

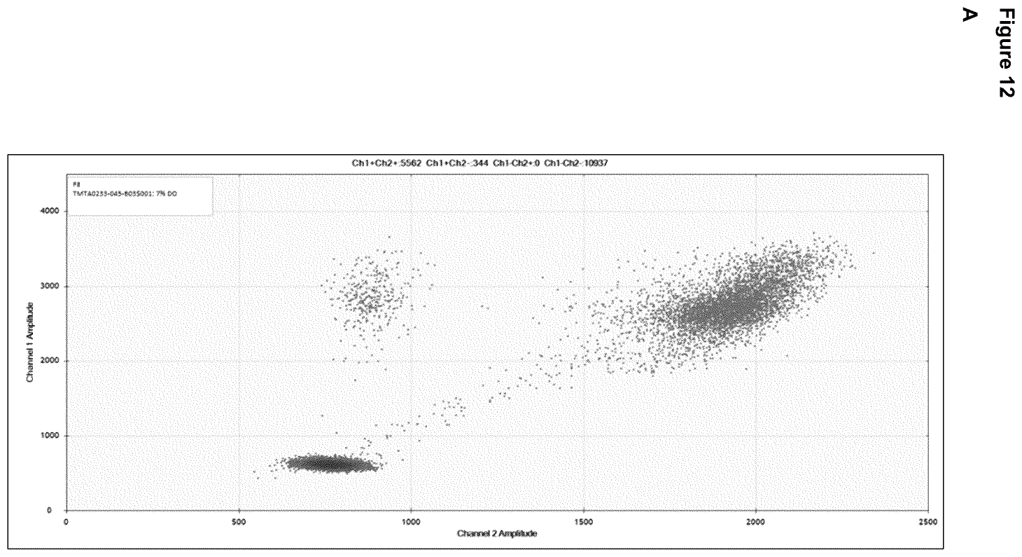

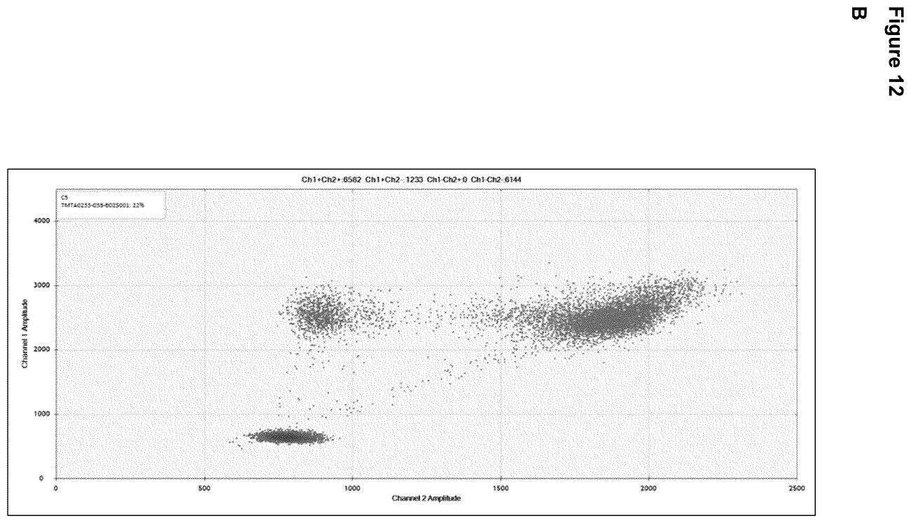

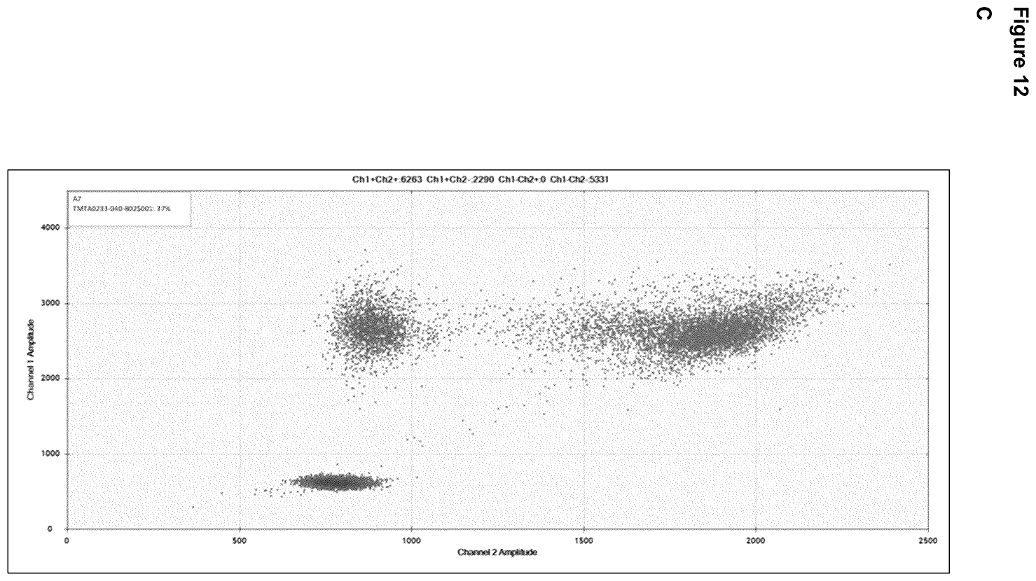

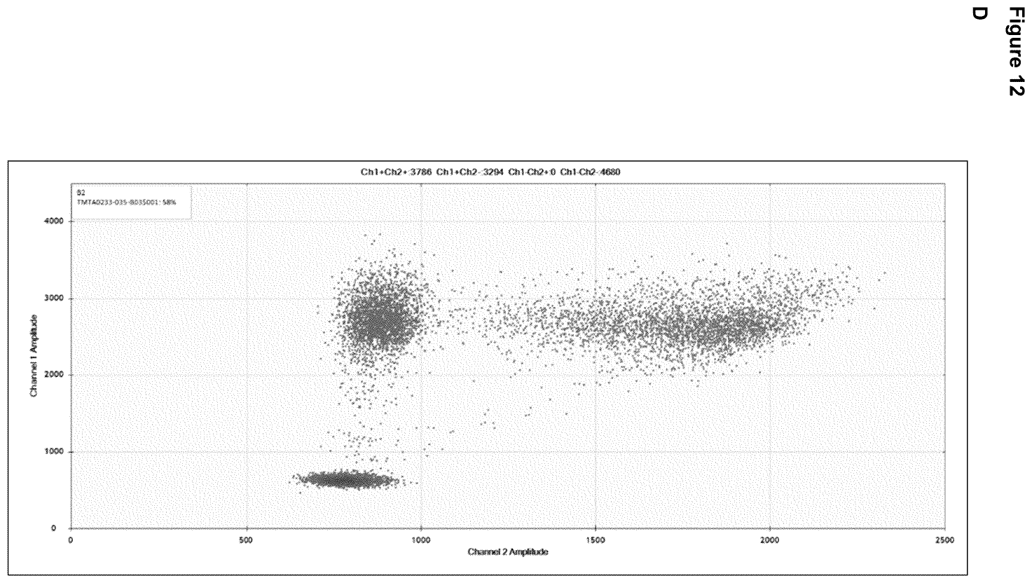

FIG. 7 shows an overview of the 3-step selection system used to identify RNP-induced indels in mlo in wheat plants. The process involves sequential enrichment for tissues carrying RNP-induced mlo INDELS through repeated sampling and molecular screening to specifically detect targeted mutations. In the example shown this was performed at 3 steps, in immature embryo explants, in regenerated populations of shoots and finally at the single plant level. A detailed overview of the selection/analysis process for Cas9 RNP is shown in FIG. 8 and FIG. 9.

Two days after biolistic RNP delivery, 5 immature embryos from each shot were selected from the inner circle area (Zone 2) and sampled for DNA isolation and ddPCR analysis to determine mlo NHEJ drop-off percentages. 20× ddPCR mixes were composed of 18 μM forward (Seq ID NO: 1) and 18 μM reverse (Seq ID NO: 2) primers, 5 μM reference probe (Seq ID NO: 3) and 5 μM drop-off probe (Seq ID NO: 5). The following reagents were mixed in a 96-well plate to make a 25-μl reaction: 11 μl of ddPCR Supermix for Probes (no dUTP), 1.1 μl of 10× assay mix (Bio-Rad Laboratories), 100-250 ng of genomic DNA in water, and water up to 22 μl. The results are summarized in Table 1, ddPCR drop-off percentages ranged from 2.0% to 5.6%.

FIG. 10 shows two-dimensional plots generated from Quantasoft Software for immature embryo samples TMTA0233-B01$001, TMTA0233-B02$001 and TMTA0233-B05$001 analyzed by ddPCR. The cluster visible in the upper-left quadrant of each plot is produced by the signal from amplicons representing NHEJ indels (RNP-targeted mlo alleles). Whereas, the cluster visible in the upper-right quadrant of each plot is produced by the signal from amplicons representing WT (non RNP-targeted mlo alleles). Samples TMTA0233-B01$001, TMTA0233-B02$001 and TMTA0233-B05$001 showed the highest drop-off percentages from all bombarded plates and as such the remaining embryos from only these shots were cultured further. The ddPCR results imply that these immature embryos are the ones most likely to carry cells with NHEJ indels in mlo alleles.

Immature embryos were passed-through 3 cycles of culture on non-selective callus induction medium 240 to obtain structured embryogenic callus from which plants could be regenerated on non-selective regeneration medium 420 in PlantCon™ containers. Leaf pieces originating from multiple shoots in each PlantCon™ container were pooled and transferred to 6 ml screw-capped tubes (Micronic MP 32301) for ddPCR analysis to determine mlo drop-off percentages. From the 3 cultured shots of TMTA0233 a total 173 pools were analyzed, originating from 62 individual embryos (63 pools from shot 1, 78 pools from shot 2 and 32 pools from shot 5). For ease of presentation the mlo drop-off percentages for each pool were classified into distinct categories (<10%, 10-25%, 25-50% and >50%). Results are shown in Table 2 and FIG. 11.

While highly variable, each of the shots contained pools showing high drop-off percentages (e.g., Shot 1:63.5% of the pools showed drop-off percentages of >25%, Shot 2: 32% of the pools showed drop-off percentages of >25% and Shot 5:46.8% of the pools showed drop-off percentages of >25%).

FIG. 12 shows two-dimensional plots generated from Quantasoft Software for representative pools showing ddPCR drop-off percentages <10% (TMTA0233-045-B03$001), 10-25% (TMTA0233-058-B02$001), 25-50% (TMTA0233-040-B02$001) and >50% (TMTA0233-035-B03$001). The cluster visible in the upper-left quadrant of each plot is produced by the signal from amplicons representing NHEJ indels (RNP-targeted mlo alleles). Whereas, the cluster visible in the upper-right quadrant of each plot is produced by the signal from amplicons representing WT (non-targeted mlo alleles).

Shoots from pools showing drop-off percentages of >50% were primarily selected for further culture (13 PlantCon™ containers from Shot 1). The ddPCR results imply that these pools are the ones most likely to carry shoots with NHEJ indels in mlo alleles. In addition, shoots from several other pools from Shot 1, showing lower drop-off percentages, were also chosen for comparison (5 PlantCon™ containers with a 25-50% drop-off percentage, 6 containers with a 10-25% drop-off percentage and 2 containers with <10% drop-off percentage).

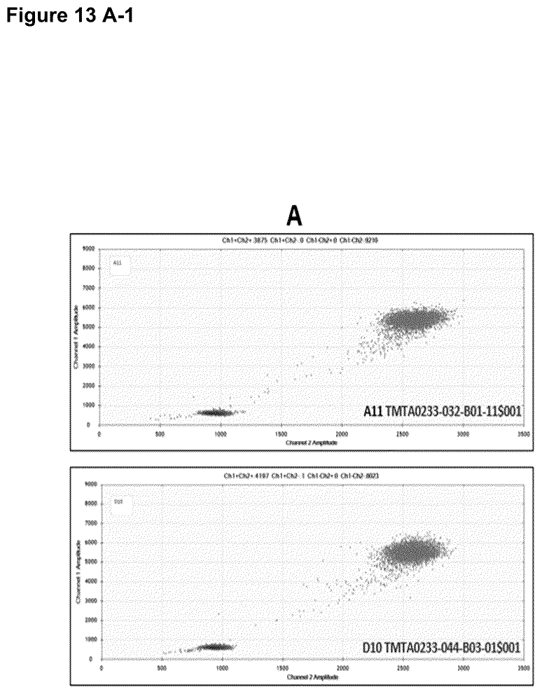

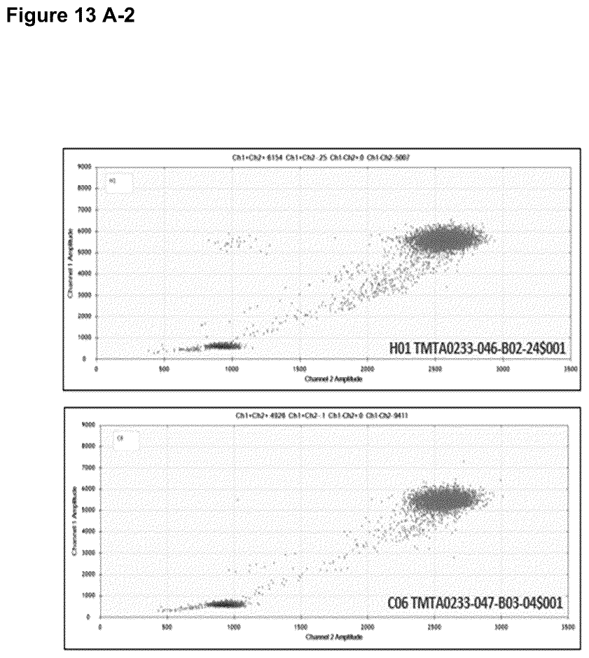

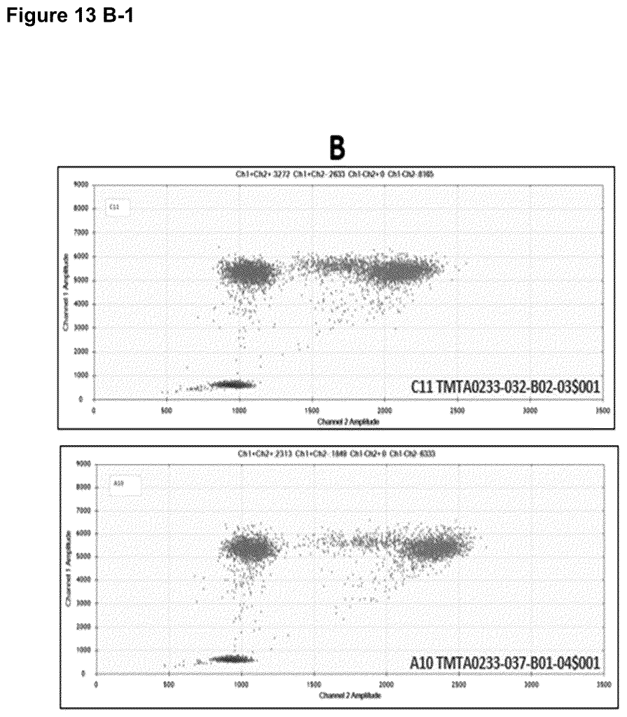

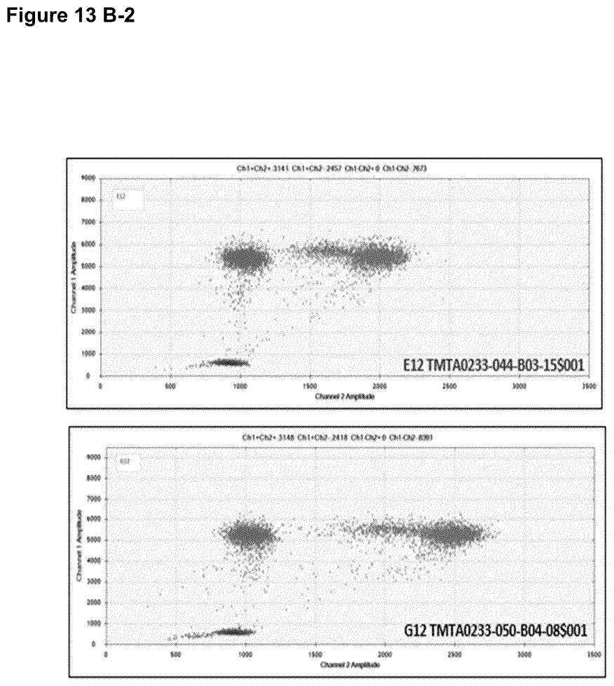

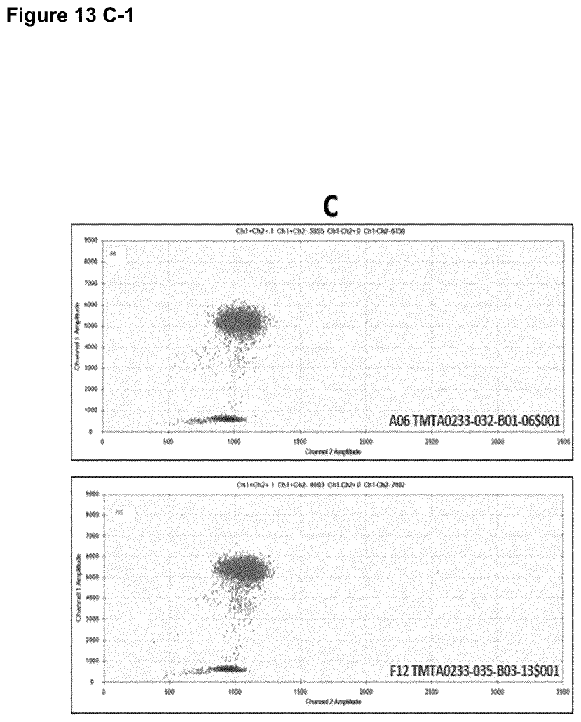

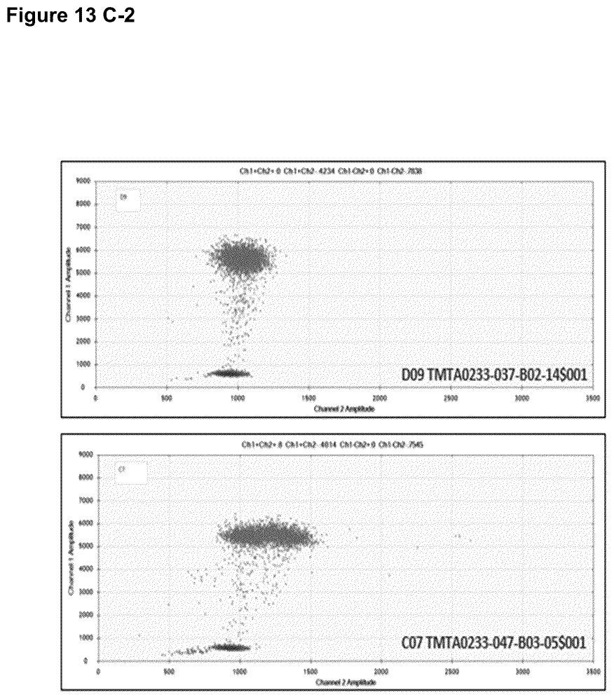

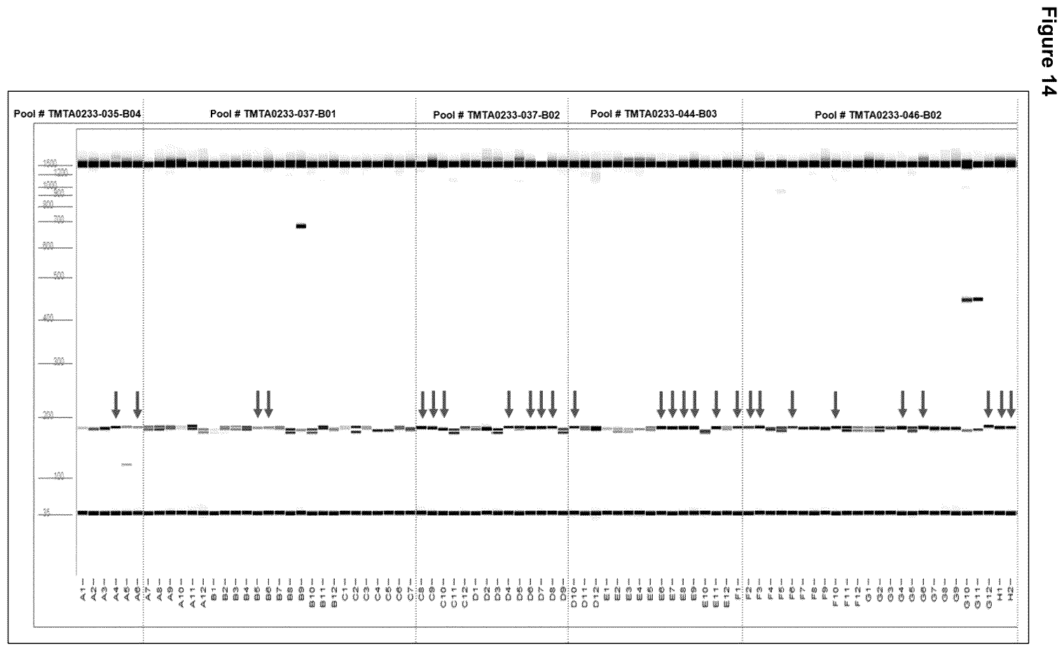

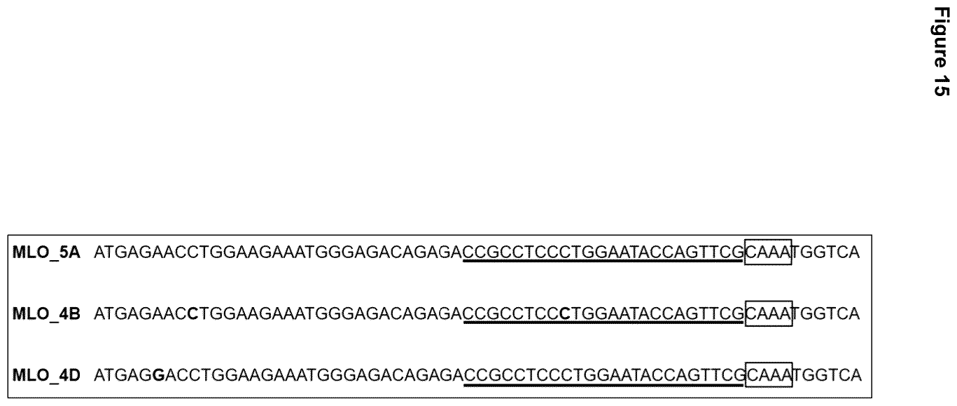

Shoots from selected containers were transferred to individual De Wit tubes containing 10 ml of non-selective rooting medium WRM for further development. Approximately 2 weeks after transfer, leaf samples from individual plants were taken for ddPCR analysis to determine mlo drop-off percentages. In total, samples were taken from 433 individual shoots originating from 17 immature embryo explants. Shoots were assigned to specific classes based on the drop-off percentages obtained with a ddPCR assay designed specifically for the A sub-genome (i.e. values close to 0%=WT, values close to 50%=monoallelic mutation and values close to 100%=biallelic mutation). The results are shown for 426 of the shoots that could be clearly assigned to specific classes (Table 3). For seven other plants mid-value drop-off percentages were observed and could not be assigned accurately to any class. Of the 426 plants classified into distinct classes, 224 plants (53%) showed drop-off percentages consistent with mlo RNP-targeting: 95 plants with drop-off percentages of 50% (=monoallelic) and 129 plants with drop-off percentages of 100% (=biallelic). FIG. 13 shows two-dimensional plots generated from Quantasoft Software for representative plants for each ddPCR drop-off percentage class (0% drop-off: TMTA0233-032-B01-11, TMTA0233-044-B03-01, TMTA0233-046-B02-24, TMTA0233-047-B03-04; 50% drop-off: TMTA0233-032-B02-03, TMTA0233-037-B01-04, TMTA0233-044-B03-15, TMTA0233-050-B04-08, and 100% drop-off: TMTA0233-032-B01-06, TMTA0233-035-B03-13, TMTA0233-037-B02-14, TMTA0233-047-B03-05). The cluster visible in the upper-left quadrant of each plot is produced by the signal from amplicons representing NHEJ indels (RNP-targeted mlo alleles). Whereas, the cluster visible in the upper-right quadrant of each plot is produced by the signal from amplicons representing WT (non-targeted mlo alleles).

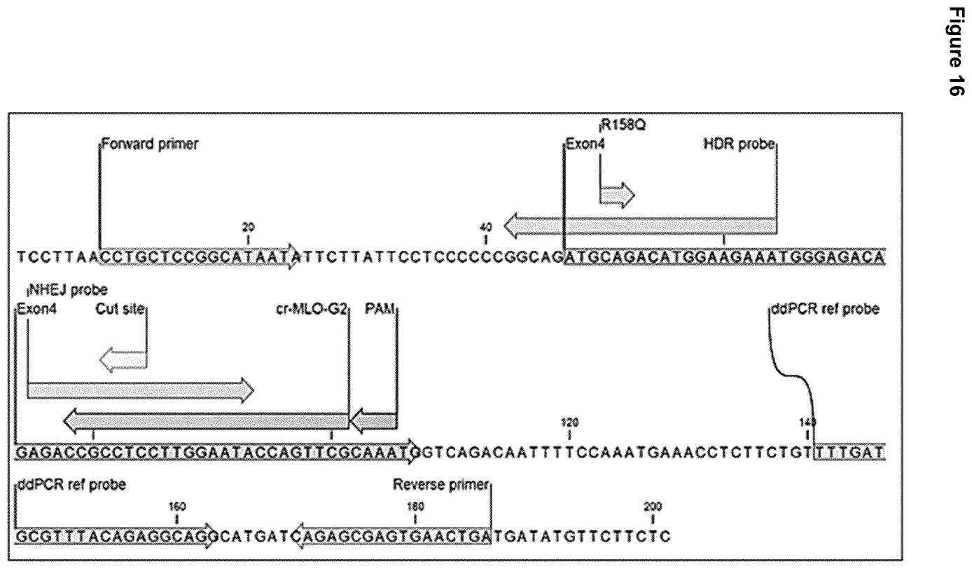

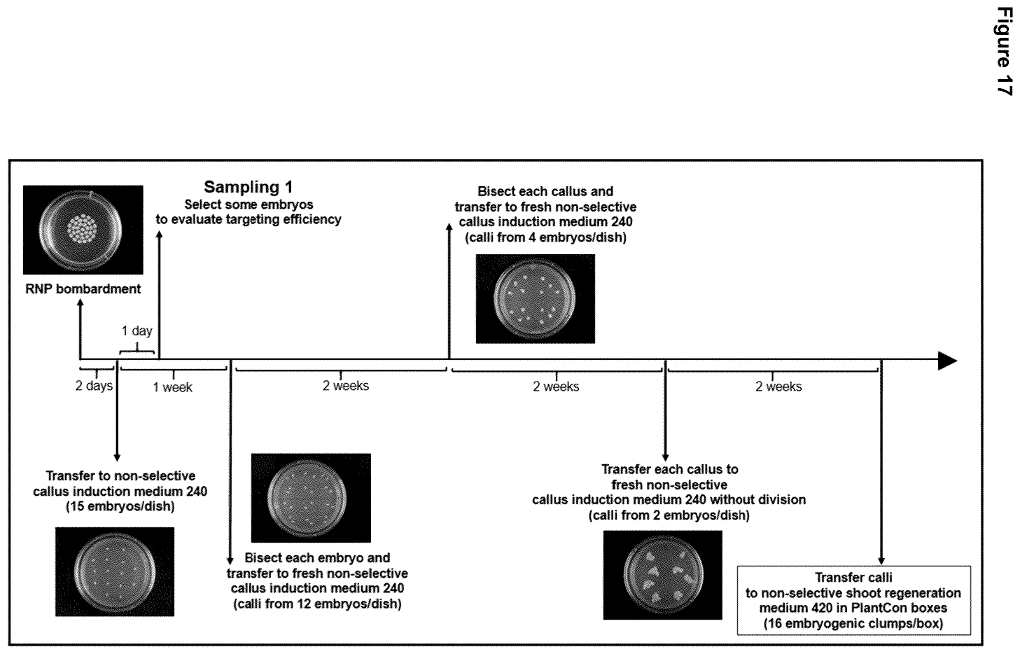

Eighty-six of the individual plants analyzed by ddPCR were further analyzed by PCR size-fragment analysis to obtain additional information about RNP-targeting of the A sub-genomic allele of mlo (primer combination Seq ID NO: 1/Seq ID NO: 2). Products (2 μl from a 50 μl total PCR volume) were separated using a Fragment Analyzer™ System (Advanced Analytical Technologies Inc, AATI). The Fragment Analyzer is a silica based capillary electrophoresis (CE) instrument with a gel matrix (DNF-920) and an intercalating dye with LED light source to easily qualify and quantify DNA fragments. Raw data analysis from the Fragment Analyzer™ was performed using PROSize® 3.0 software (AATI) to provide information on the size and concentration of the separated DNA fragments. In total, 6 plants derived from pool TMTA0233-035-B04, 25 plants from pool TMTA0233-037-B01, 14 plants from pool TMTA0233-037-B02, 16 plants from pool TMTA0233-044-B03 and 25 plants from pool TMTA0233-046-B02 (FIG. 14) were analyzed. In the individual plants derived from each analyzed pool, a considerable number of plants predicted by the ddPCR drop-off analysis to carry mlo mutations, showed variation in fragment length (often with multiple fragments). In contrast, multiple fragments were not observed in plants that showed 0% drop-off during ddPCR analysis (27 plants marked with arrows). Interestingly, different fragment patterns were observed within the individual plants analyzed from a single pool, suggesting that not all plants analyzed were clonal.

Twenty-nine of the individual plants analyzed by PCR size-fragment analysis were further analyzed by NGS to obtain precise sequence information for the A and additionally B and D mlo sub-genomic copies (Table 4). Plants selected for NGS included 7 plants, predicted by ddPCR to be WT for both A mlo sub-genome alleles, to check if additional mutations were present on the B and D sub-genomic copies. 500 ng of purified PCR products (primer combination Seq ID NO: 11/Seq ID NO: 12) were sent for NGS (GENEWIZ® Germany GmbH). The Amplicon-EZ Illumina-based service provides full sequence coverage of PCR products up to 500 bp in length (up to 50.000 reads/sample are delivered). The raw Illumina data was analyzed and visualized using the software application CLC Genomics Workbench 12.0.3 (Qiagen) and proprietary CRISPRMapper plugin. Overall the results generally showed a close correlation between the assigned class for A sub-genome drop-off percentage and NGS e.g., TMTA0233-032-B01-08 showed a 0% drop-off for the ddPCR assay and both A sub-genome mlo alleles were confirmed by NGS to be WT, TMTA0233-035-B03-07 showed a 52% drop-off for the ddPCR assay and one of the mlo alleles was confirmed by NGS to carry a deletion of 5 bp, while the other allele was WT (monoallelic), TMTA0233-047-B01-01 showed a 100% drop-off for the ddPCR assay and here one mlo allele was confirmed by NGS to carry a deletion of 2 bp and the other a 10 bp deletion (biallelic). For plants where there was no correlation between the assigned class for A sub-genome drop-off percentage and the NGS results, it was observed that at least one of the A sub-genome alleles carried an insertion of +1 bp that may be insufficient to prevent binding of the NHEJ drop-off probe. NGS results also showed that the B and D sub-genomic copies of mlo were also targeted efficiently with some plants carrying INDELS in all 6 mlo copies (e.g., TMTA0233-032-B01-06, TMTA0233-035-B04-01 and TMTA0233-044-B03-13). Targeting of the B sub-genomic allele of mlo was not expected to be so efficient given that the gRNA used has a 1 bp mismatch to the target sequence.

To provide data on the number of unique mutant plants that could be recovered from one bombarded immature embryo, 23 plants from explant TMTA0233-037 were analyzed by NGS: 7 previously characterized as 50% ddPCR drop-off for the A sub-genomic allele of mlo, 7 previously characterized as 100% ddPCR drop-off, 5 plants previously characterized as 0% ddPCR drop-off and 4 plants with mid-value ddPCR drop-off percentages. NGS results showed that 14 of the 23 plants selected from the single embryo explant carried unique mutation profiles (Table 5).

### Analysis of Progeny Populations

To provide data on inheritance of Cas9-induced mutations, 30 seeds from 3 self-pollinated plants (TMTA0233-032-B01-06, TMTA0233-035-B04-01 and TMTA0233-044-B03-13), previously characterized to carry 6 targeted mlo alleles were sown in the greenhouse, sampled and analyzed by NGS to determine indel transmission. All predicted mutations were recovered in the 3 progeny populations and segregated as expected (Table 11, Table 12 and Table 13).

### Example 2-Cas12a RNP Targeting of mlo

gRNA Design for Cas 12a Targeting of mlo

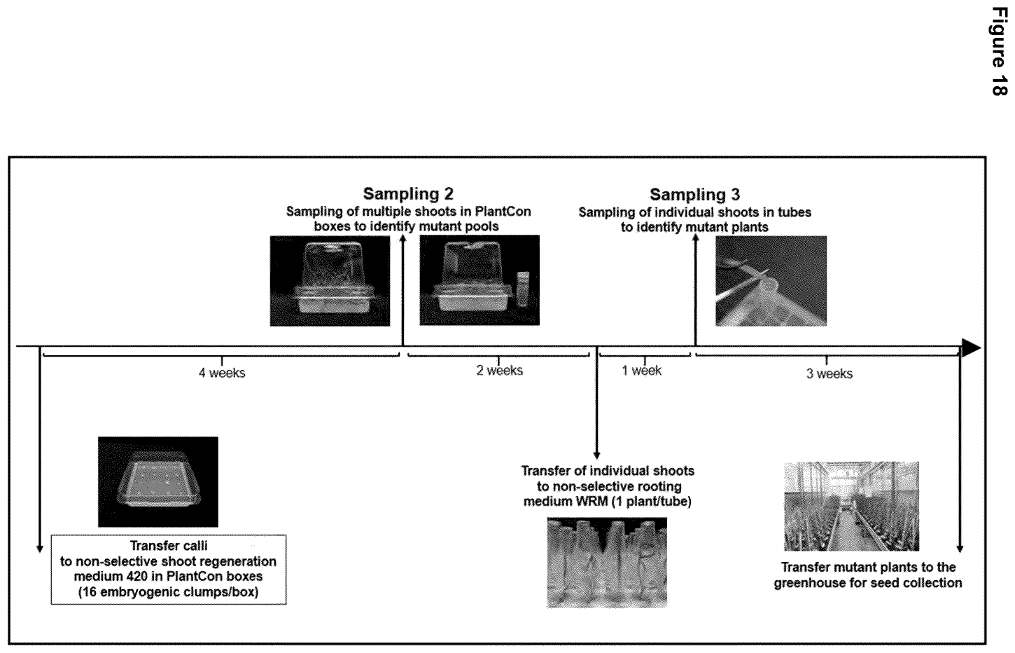

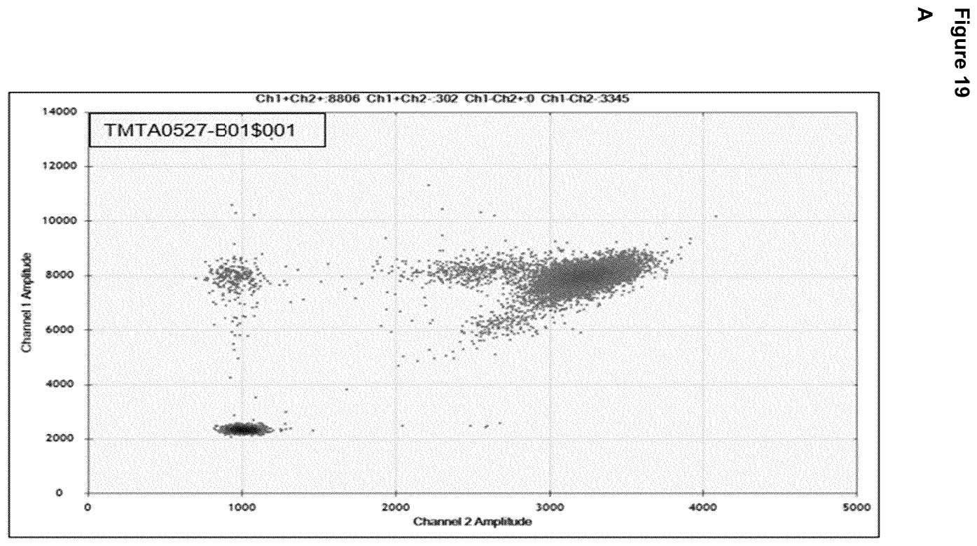

Building upon previous work (Gil-Humanes et al. 2017) we designed a synthetic crRNA composed of a 21 bp direct repeat sequence and a 24 bp protospacer targeting the wheat mildew resistance locus O (mlo). The mlo gene encodes a seven-transmembrane domain protein involved in resistance to the fungal pathogen Blumeria graminis. The recognition site of the crR-NA is located on the antisense strand within exon 4 of mlo [5′-[(TTTG)CGAACTGGTATTCCAAGGAGGCGG-3′], with PAM site between brackets. The designed guide is specific for the 5A and 4D alleles of mlo and shows one mismatch with the 4B allele at position 16 from the PAM sequence (FIG. 15).

Droplet Digital PCR (ddPCR) Assay to Identify Cas12a RNP-Induced mlo Indel Mutations

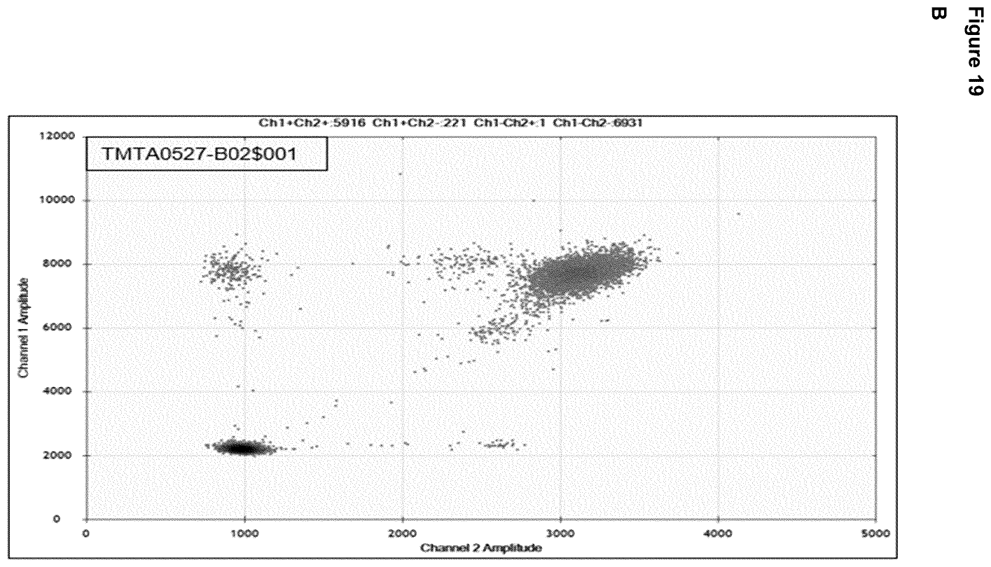

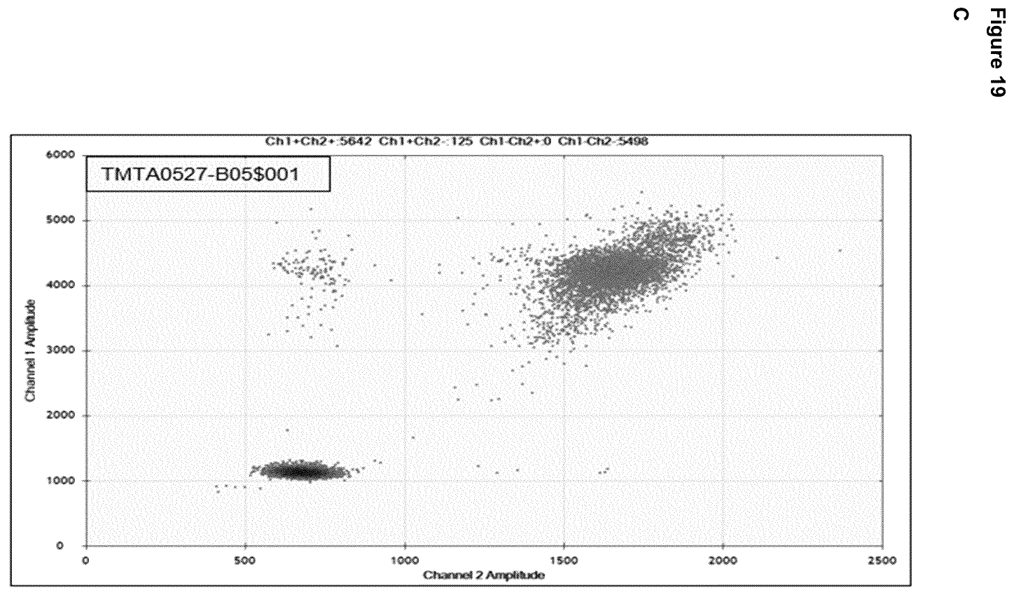

The ddPCR assay used to identify Cas 12a-RNP induced mlo indels was the same as that previously described for the detection of Cas9-RNP induced mlo indels (for details of the ddPCR design see Example 1-Cas9 RNP targeting of mlo). The location of the predicted Cas 12a cut site in the mlo gene and the position of the PCR primers and probes used for the ddPCR analysis are shown in FIG. 16.

### Wheat Donor Plant Growth for Immature Embryo Isolation

Donor plants of cv. Fielder were grown under controlled-environment conditions to ensure optimal immature embryo quality. Typical growth conditions were day/night temperatures of 20° C.+/−1° C. and 18° C.+/−1° C., 65% relative humidity with a 16 h photoperiod (400 μmol/m²/s illumination at table level) provided by a mixture of 600W high pressure sodium lamps and 400W metal halide lamps.

### Preparation of Wheat Immature Embryos for Bombardment

Immature seeds were harvested from donor plants containing embryos around 2 mm in length, peeled and sterilized in 70% v/v ethanol for 1 min and followed by 10 min in 10% v/v sodium hypochlorite (ACROS bleach containing 10-15% active chlorine). Finally, the immature seeds were washed several times with demineralized water.

Embryos were aseptically excised from the immature seeds using a binocular microscope (Model MZ6, Leica) taking care to slice-off the embryo axis through the green seed coat during preparation. Subsequent steps for embryo preparation were performed using a modified procedure essentially as described by Ishida et al. (2015). Immature embryo explants were transferred to 3.5 cm Petri dishes (Falcon 351008) containing 4.5 ml of non-selective callus induction medium WLS, herewith designated callus induction medium 240. Embryos were arranged in a central circle of 1.5 cm (25-50 embryos/dish with the scutellum side facing upwards).

### Preparation of Cas 12a RNP Complexes

Purified Cas12a nuclease and mlo-specific crRNA were ordered from IDT (Integrated DNA Technologies) for RNP assembly.
crRNA: custom and user-defined crRNA that binds to 24 bases on the DNA strand that is opposite to the TTTV, PAM sequence
Cas12a nuclease: (L.b. Cas12a Ultra Nuclease): Recombinant Lachnospiraceae bacterium Cas 12a nuclease, purified from E. coli strain expressing Cas12a. Contains C-terminal nuclear localization signal (NLS) and C-terminal 6-His tag.

For RNP complex assembly, mlo-specific crRNA was mixed with Cas 12a nuclease in NEBuffer™ 2.1 (New England BioLabs) in an approximate equimolar ratio. The mixture was incubated at 37° C. for 20-30 min and then transferred to ice.

Biolistic Delivery of Cas 12a RNP Complexes into Wheat

RNP delivery into immature embryos was conducted using the Biolistic®PDS-1000/He Particle Delivery System (Bio-Rad) using a modified procedure of Liang et al. (2018) adapted for Cas12a RNP delivery. RNP complexes were mixed with 0.6 μm gold particles (BioRad) together with TransIT-CRISPR® transfection reagent (Sigma-Aldrich), 15 μl aliquots were spread over the central region of each macrocarrier and airdried for 30 min in a laminar flow bench prior to delivery. Typically, for each shot 150 μg of gold particles and 3 μg of Cas 12a protein complexed with crRNA were delivered.

### Culture of Bombarded Wheat Immature Embryos and Plant Regeneration

Following bombardment, the immature embryos were incubated on the same plates for 48 h in the dark (28° C.+/−1° C., 55% relative humidity in an MLR-352H-PE Panasonic incubator). After this period embryos were transferred to fresh non-selective callus induction medium 240 (9 cm dishes containing 35-40 ml of medium with up to 15 embryos/dish) and incubated in the dark (25° C.+/−1° C.). At this stage, embryos from different positions in the target area were separated (FIG. 5) i.e. from the center (Zone 1), inner circle (Zone 2) and outer circle area (Zone 3).

Seven days after bombardment immature embryos were longitudinal bisected into 2 pieces under a binocular microscope and transferred to fresh non-selective callus induction medium 240 (9 cm dishes containing 35-40 ml of medium, 12 embryos/dish) and cultured in the dark (25° C.+/−1° C.).

After 2 weeks, the bisected immature embryos were once more bisected under a binocular microscope and transferred to fresh non-selective callus induction medium 240 (9 cm dishes containing 35-40 ml of medium, 4-6 embryos/dish) and cultured in the dark under the same conditions. After a further 2 weeks, the calli from each embryo were transferred intact to fresh nonselective callus induction medium 240 (9 cm dishes containing 35-40 ml of medium, 2 embryos/dish) and cultured in the dark under the same conditions.

Two weeks later, small pieces of structured embryogenic callus tissue were transferred to a non-selective regeneration medium, LSZ (Ishida et al., 2015), herewith designated regeneration medium 420. Regeneration medium 420 was prepared in PlantCon™™ containers (MP Biomedicals, Catalogue Nr. 26-722-06), 100 ml medium/container. The embryogenic calli arising from one embryo were transferred to one PlantCon™ container (max. 16 pieces/container). Where more than 16 embryogenic calli were recovered from one embryo, additional regeneration containers were utilized. PlantCon™ containers were incubated in the light for approximately 6 weeks (23° C.+/−1° C., 16 h photoperiod).

Shoots from PlantCon™ containers were transferred individually to De Wit tubes (Duchefa Biochemie) containing 10 ml of non-selective rooting medium WRM, essentially a modification of medium R (Sparks and Jones, 2009), solidified with 0.15% w/v Gelrite (Duchefa Biochemie) and cultured in the light (23° C.+/−1° C., 16 h photoperiod). Rooted plants were transferred to the greenhouse.

### Tissue Sampling and DNA Isolation

Sampling 1-Three days after bombardment, 5 immature embryos were randomly selected from Zone 2 (FIG. 5) of each bombarded plate. The embryos were bulked as one sample, collected in 2 ml tubes (Eppendorf® Safe-Lock) and stored at −80°. Samples were ground in a Retsch Mixer Mill MM300/400 for 60 sec. Genomic DNA extraction was performed using the Qiagen Dneasy Plant Mini Kit (Catalogue Nr. 69106) according to the manufacturer's instructions. Final DNA concentrations were measured, and plates stored at 4° C. until use for downstream analysis.

Sampling 2-Following 4 weeks of culture of structured embryogenic callus on non-selective regeneration medium 420 in PlantCon™ containers, leaf pieces were harvested from regenerating shoots. Care was taken to ensure that similar sized leaf pieces were taken from all regenerating shoots, this was best achieved by first cutting off all longer leaf tips with sterile scissors and then cutting leaf pieces of approximately 5 mm in length from the remaining tissues (FIG. 6). Leaf pieces originating from each PlantCon™ container were pooled and transferred to 6 ml screw-capped tubes (Micronic MP 32301) and stored at −80°. Samples were freeze dried overnight and genomic DNA extracted using a procedure based on the LGC GENOMICS sbeadex™ Maxi Plant Kit with KingFisher automation. Final DNA concentrations were measured, and plates stored at 4° C. until use for downstream analysis.

Sampling 3-Following 1-2 weeks of culture of individual shoots in De Wit tubes containing 10 ml of WRM medium, leaf pieces were harvested from shoots. Samples were collected in 1.4 ml push-cap tubes in a 96-sample carrier rack (Micronic MP 226RP) and stored at −80°. Genomic DNA was extracted using a procedure based on the LGC GENOMICS sbeadex™ Maxi Plant Kit with KingFisher automation. Final DNA concentrations were measured, and plates stored at 4° C. until use for downstream analysis.

### Selection/Analysis Process to Identify Cas12a RNP Indels in mlo

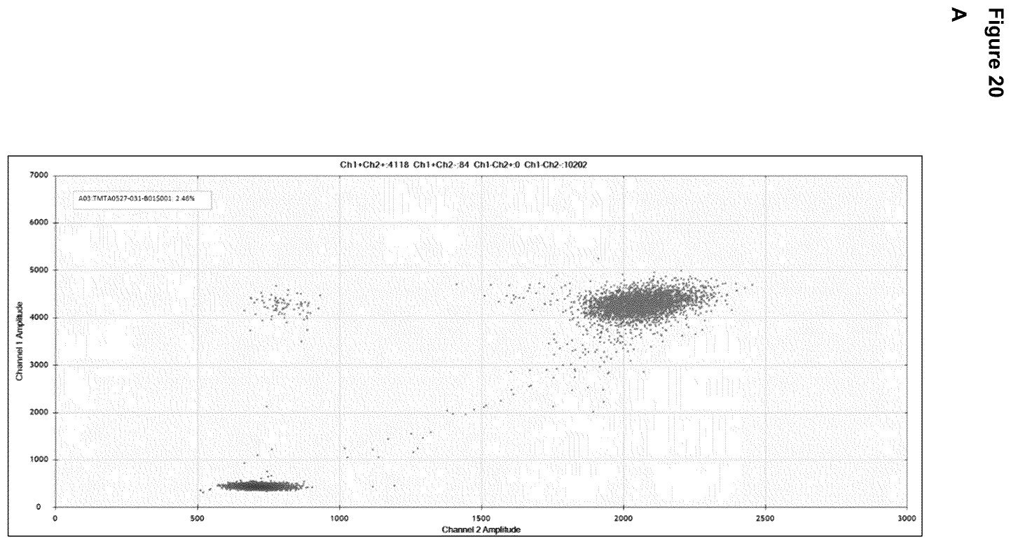

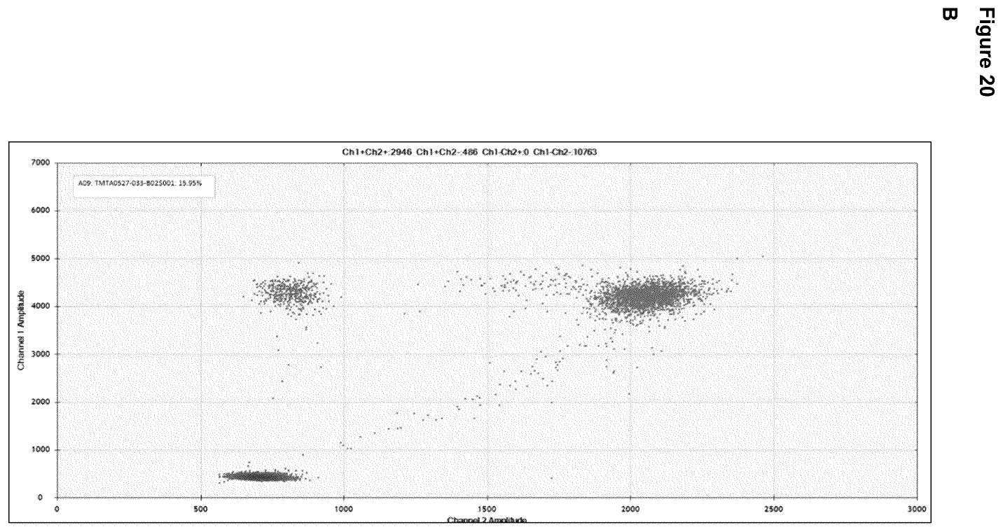

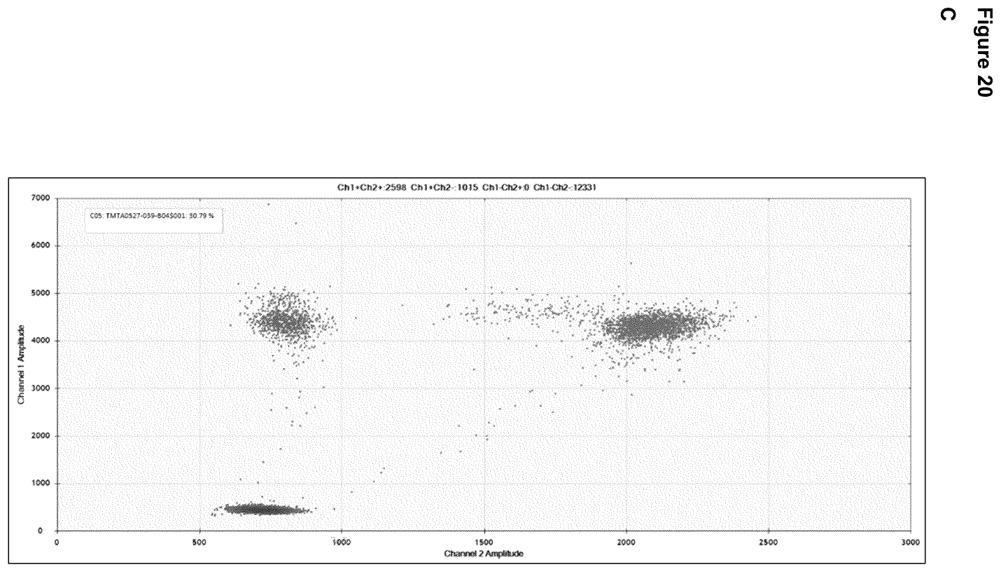

FIG. 7 shows an overview of the 3-step selection system used to identify RNP-induced indels in mlo in wheat plants. The process involves sequential enrichment for tissues carrying RNP-induced mlo INDELS through repeated sampling and molecular screening to specifically detect targeted mutations. In the example shown this was performed at 3 steps, in immature embryo explants, in regenerated populations of shoots and finally at the single plant level. A detailed overview of the selection/analysis process for Cas12a RNP is shown in FIG. 17 and FIG. 18.

Three days after biolistic RNP delivery, 5 immature embryos from each shot were selected from the inner circle area (Zone 2) and sampled for DNA isolation and ddPCR analysis to determine mlo NHEJ drop-off percentages. 20× ddPCR mixes were composed of 18 μM forward (Seq ID NO: 1) and 18 μM reverse (Seq ID NO: 2) primers, 5 μM reference probe (Seq ID NO: 3) and 5 μM drop-off probe (Seq ID NO: 5). The following reagents were mixed in a 96-well plate to make a 25-μl reaction: 11 μl of ddPCR Supermix for Probes (no dUTP), 1.1 μl of 10× assay mix (Bio-Rad Laboratories), 100-250 ng of genomic DNA in water, and water up to 22 μl. The results are summarized in Table 6, ddPCR drop-off percentages ranged from 0.5% to 2.7%.

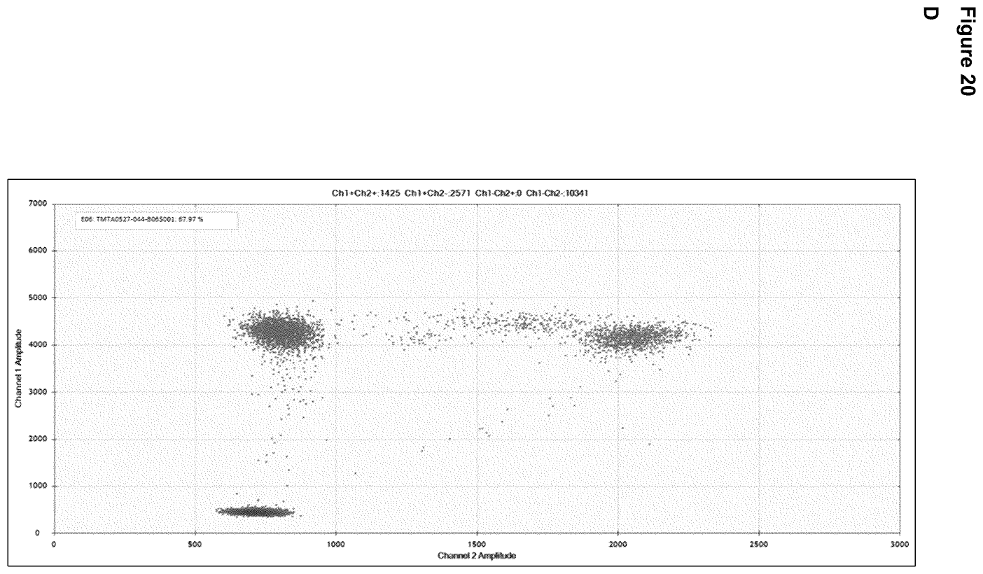

FIG. 19 shows two-dimensional plots generated from Quantasoft Software for immature embryo samples TMTA0527-B01$001, TMTA0527-B02$001 and TMTA0527-B05$001 analyzed by ddPCR. The cluster visible in the upper-left quadrant of each plot is produced by the signal from amplicons representing NHEJ indels (RNP-targeted mlo alleles). Whereas, the cluster visible in the upper-right quadrant of each plot is produced by the signal from amplicons representing WT (non RNP-targeted mlo alleles). Samples TMTA0527-B01$001, TMTA0527-B02$001 and TMTA0527-B05$00 showed the highest drop-off percentages from all bombarded plates and as such the remaining embryos from only these shots were cultured further. The ddPCR results imply that these immature embryos are the ones most likely to carry cells with NHEJ indels in mlo alleles.

Immature embryos were passed-through 3 cycles of culture on non-selective callus induction medium 240 to obtain structured embryogenic callus from which plants could be regenerated on non-selective regeneration medium 420 in PlantCon™ containers. Leaf pieces originating from multiple shoots in each PlantCon™ container were pooled and transferred to 6 ml screw-capped tubes (Micronic MP 32301) for ddPCR analysis to determine mlo drop-off percentages. From the 3 cultured shots of TMTA0527 a total 139 pools were analyzed, originating from 46 individual embryos (58 pools from shot 1, 31 pools from shot 2 and 50 pools from shot 5). For ease of presentation the mlo drop-off percentages for each pool were classified into distinct categories (<10%, 10-25%, 25-50% and >50%). Results are shown in Table 7. While highly variable, each of the shots contained pools showing high drop-off percentages (e.g., Shot 1:27.6% of the pools showed drop-off percentages of >25%, Shot 2: 58% of the pools showed drop-off percentages of >25% and Shot 5: 8% of the pools showed drop-off percentages of >25%).

FIG. 20 shows two-dimensional plots generated from Quantasoft Software for representative pools showing ddPCR drop-off percentages <10% (TMTA0527-031-B01$001), 10-25% (TMTA0527-033-B02$001), 25-50% (TMTA0527-039-B04$001) and >50% (TMTA0527-044-B06$001). The cluster visible in the upper-left quadrant of each plot is produced by the signal from amplicons representing NHEJ indels (RNP-targeted mlo alleles). Whereas, the cluster visible in the upper-right quadrant of each plot is produced by the signal from amplicons representing WT (non-targeted mlo alleles).

Shoots from pools showing drop-off percentages of >25% were primarily selected for further culture (13 PlantCon™ containers from Shot 1, 8 PlantCon™ containers from Shot 2, 10 PlantCon™ containers from Shot 5). The ddPCR results imply that these pools are the ones most likely to carry shoots with NHEJ indels in mlo alleles. In addition, shoots from 7 other pools showing lower drop-off percentages (class 10-25%) were also selected.

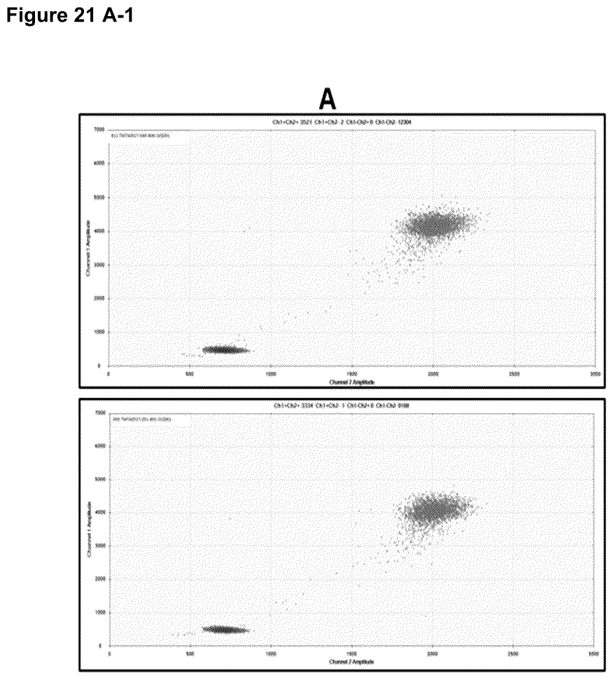

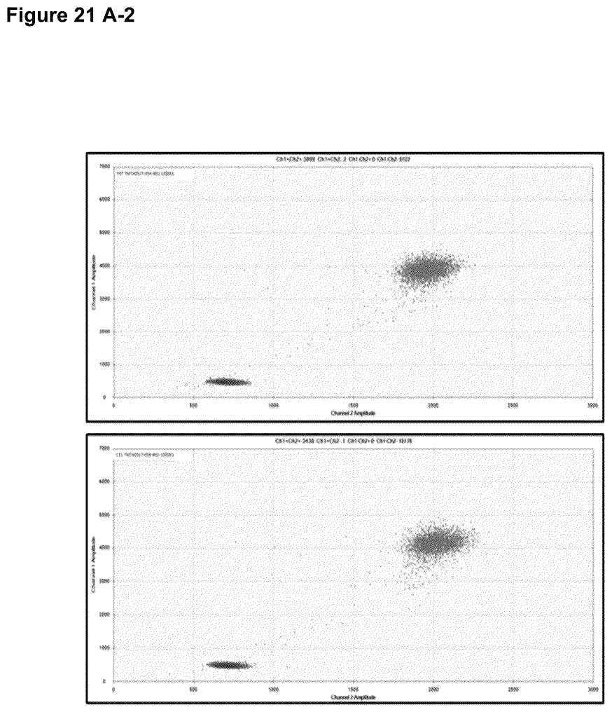

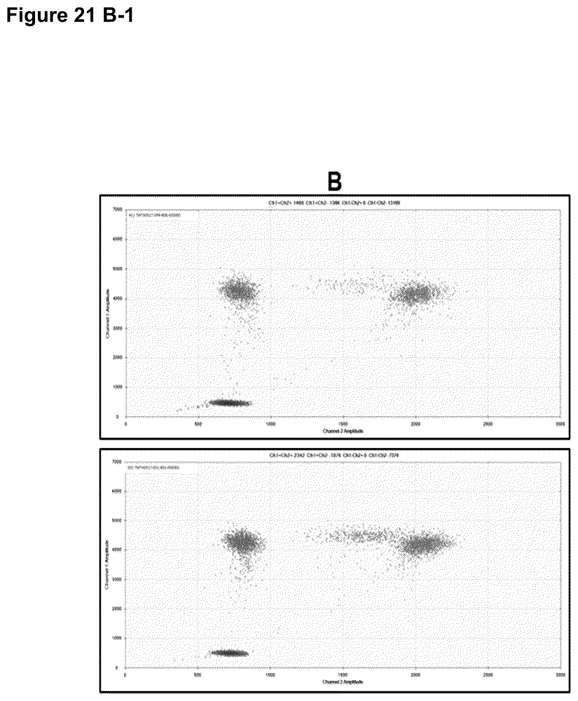

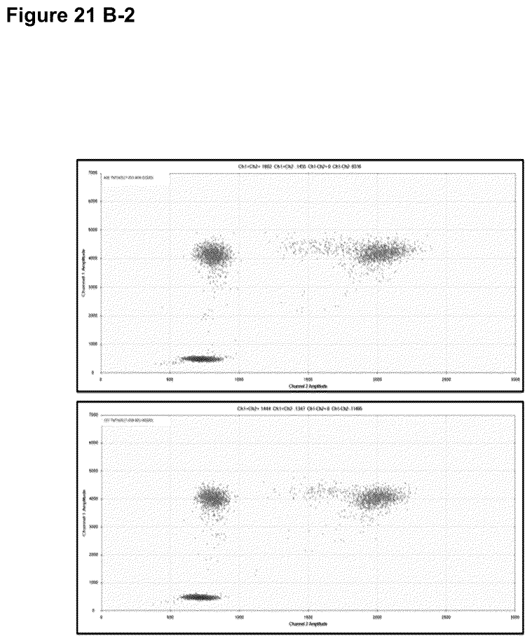

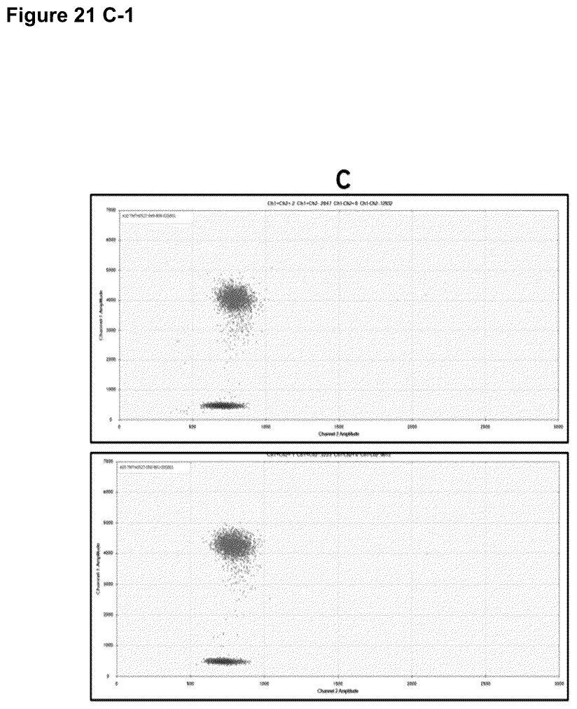

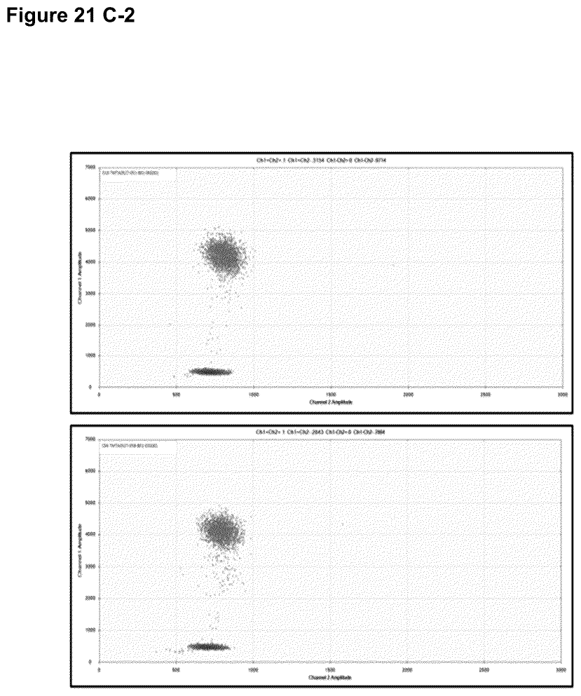

Shoots from selected containers were transferred to individual De Wit tubes containing 10 ml of non-selective rooting medium WRM for further development. Approximately 2 weeks after transfer, leaf samples from individual plants were taken for ddPCR analysis to determine mlo drop-off percentages. In total, samples were taken from 440 individual shoots originating from 24 immature embryo explants. Shoots were assigned to specific classes based on the drop-off percentages obtained with a ddPCR assay designed specifically for the A sub-genome (i.e. values close to 0%=WT, values close to 50%=monoallelic mutation and values close to 100%=biallelic mutation). The results are shown for 436 of the shoots that could be clearly assigned to specific classes (Table 8). For four other plants mid-value drop-off percentages were observed and could not be assigned accurately to any class. Of the 436 plants classified into distinct classes, 224 plants (51%) showed drop-off percentages consistent with mlo RNP-targeting: 155 plants with drop-off percentages of 50% (=monoallelic) and 69 plants with drop-off percentages of 100% (=biallelic). FIG. 21 shows two-dimensional plots generated from Quantasoft Software for representative plants for each ddPCR drop-off percentage class (0% drop-off: TMTA0527-049-B06-16, TMTA0527-051-B01-01, TMTA0527-054-B01-10, TMTA0527-058-B01-10; 50% drop-off: TMTA0527-049-B06-03, TMTA0527-051-B01-09, TMTA0527-053-B04-02, TMTA0527-058-B01-06, and 100% drop-off: TMTA0527-049-B06-02, TMTA0527-050-B01-03, TMTA0527-051-B01-06, TMTA0527-058-B01-03). The cluster visible in the upper-left quadrant of each plot is produced by the signal from amplicons representing NHEJ indels (RNP-targeted mlo alleles). Whereas, the cluster visible in the upper-right quadrant of each plot is produced by the signal from amplicons representing WT (non-targeted mlo alleles).

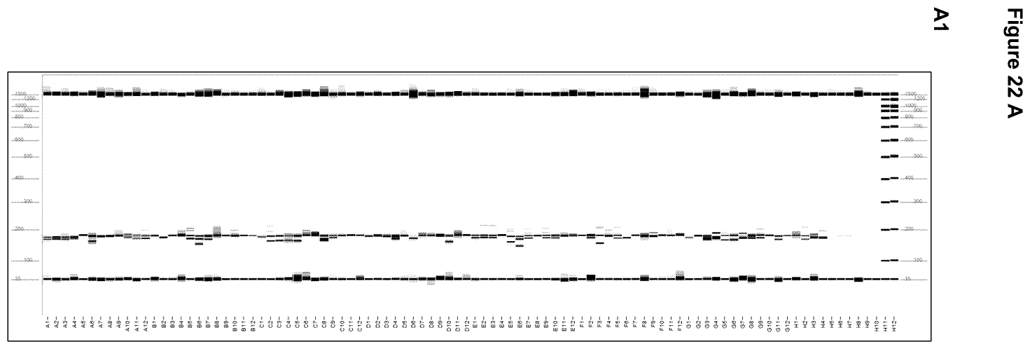

Individual plants analyzed by ddPCR were further analyzed by PCR size-fragment analysis to obtain additional information about RNP-targeting of the A sub-genomic allele of mlo (primer combination Seq ID NO: 1/Seq ID NO: 2). Products (2 μl from a 50 μl total PCR volume) were separated using a Fragment Analyzer™ System (Advanced Analytical Technologies Inc, AATI). The Fragment Analyzer is a silica based capillary electrophoresis (CE) instrument with a gel matrix (DNF-920) and an intercalating dye with LED light source to easily qualify and quantify DNA fragments. Raw data analysis from the Fragment Analyzer™ was performed using PROSize® 3.0 software (AATI) to provide information on the size and concentration of the separated DNA fragments (FIG. 22). Considerable variation in fragment length was observed (often with multiple fragments), within the plants recovered from each pool. Twenty-one of the individual plants analyzed by PCR size-fragment analysis were selected for NGS to obtain precise sequence information for the A and additionally B and D mlo sub-genomic copies (Table 9). Plants selected for NGS included 4 plants, predicted by ddPCR to be WT for both A sub-genome mlo alleles, to check if additional mutations are present on the B and D sub-genomic copies). 500 ng of purified PCR products (primer combination Seq ID NO: 11/Seq ID NO: 12) were sent for NGS (GENEWIZ® Germany GmbH). The Amplicon-EZ Illumina-based service provides full sequence coverage of PCR products up to 500 bp in length (up to 50.000 reads/sample are delivered). The raw Illumina data was analyzed and visualized using the software application CLC Genomics Workbench 12.0.3 (Qiagen) and proprietary CRISPRMapper plugin. Overall the results generally showed a close correlation between the assigned class for A sub-genome drop-off percentage and NGS e.g., TMTA0527-049-B06-07 showed a 0% drop-off for the ddPCR assay and both A sub-genome mlo alleles were confirmed by NGS to be WT, TMTA0527-049-B06-03 showed a 51% drop-off for the ddPCR assay and one of the mlo alleles was confirmed by NGS to carry a deletion of 10 bp, while the other allele was WT (monoallelic), TMTA0527-049-B06-17 showed a 100% drop-off for the ddPCR assay and here one mlo allele was confirmed by NGS to carry a deletion of 3 bp and the other a 6 bp deletion (biallelic). For one plant analyzed, where there was no correlation between the assigned class for A sub-genome drop-off percentage and the NGS results, it was observed that one of the A sub-genome alleles carried an insertion of 1 bp that may be insufficient to prevent binding of the NHEJ drop-off probe (TMTA0527-044-B06-16). NGS results also showed that the B and D sub-genomic copies of mlo were also targeted, however targeting of the B sub-genomic allele of mlo was less efficient, presumably due to the +1 bp mismatch of the Cas 12a gRNA to the target sequence.

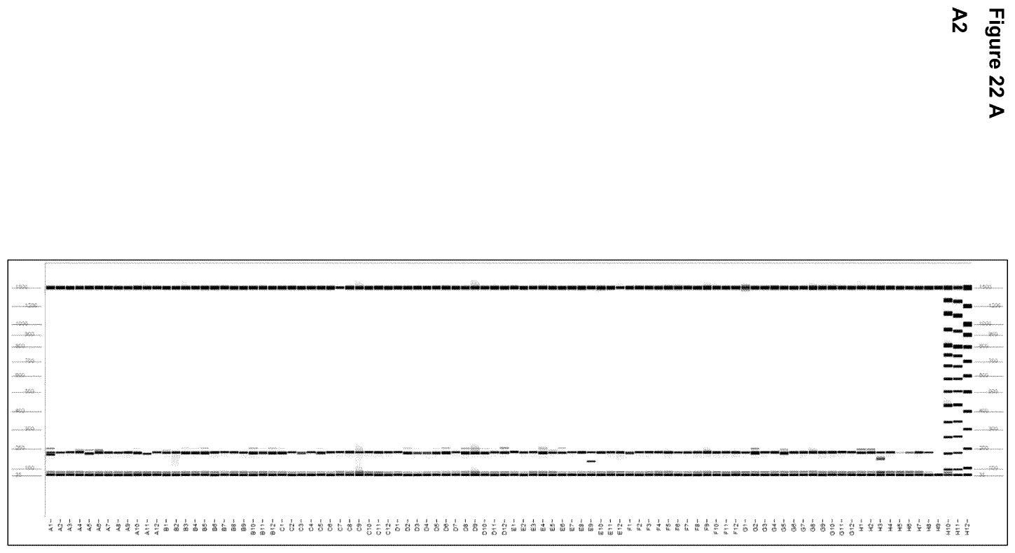

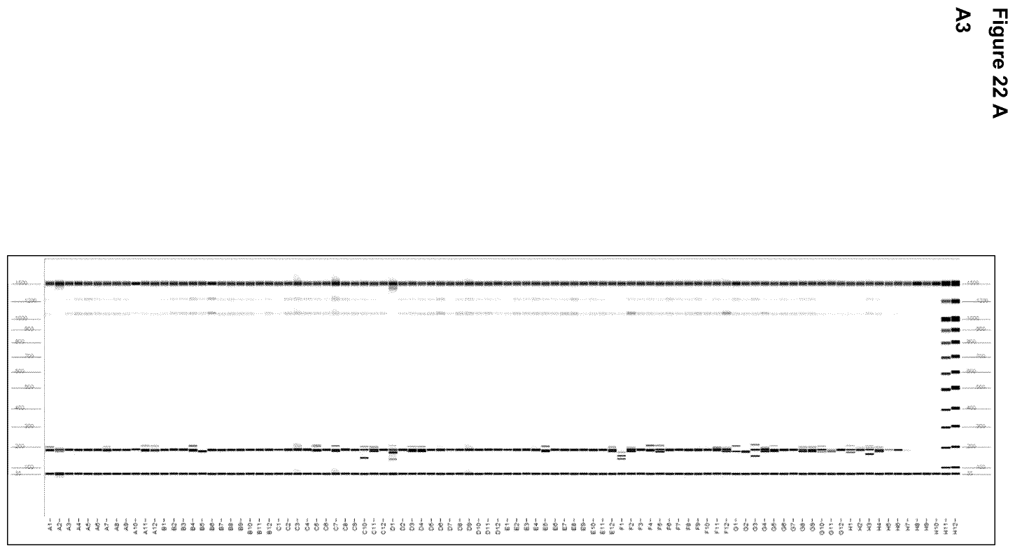

To provide data on the number of unique mutant plants that could be recovered from one bombarded immature embryo (TMTA0527-045), 35 from 62 plants analyzed by PCR size-fragment analysis (FIG. 23) were selected for NGS: 15 previously characterized as 50% ddPCR drop-off for the A sub-genomic allele of mlo, 15 previously characterized as 100% ddPCR drop-off and 5 plants previously characterized as 0% ddPCR drop-off. Plants were selected showing distinct fragment patterns (variation in length and number). NGS results showed that 32 of the 35 plants selected from the single embryo explant carried unique mutation profiles (Table 10).

### Example 3-Cas 12a RNP Targeting of FLC

gRNA design for Cas12a targeting of FLC

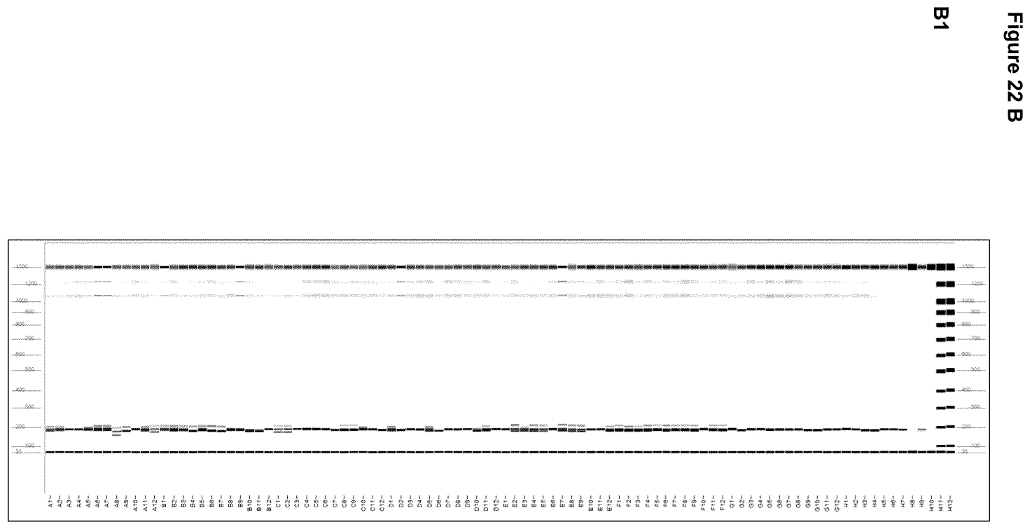

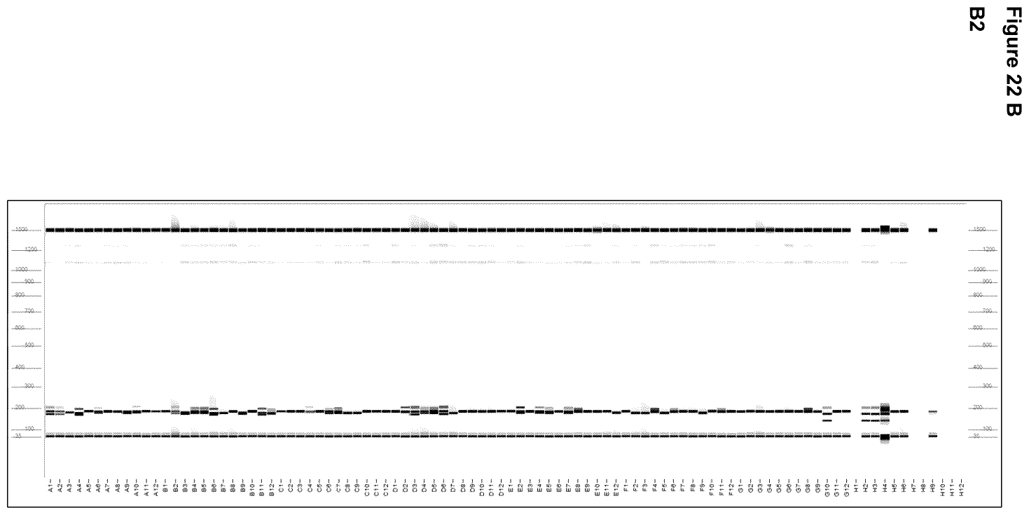

FLOWERING LOCUS C (FLC) genes are known to have a key role in the vernalization response in wheat, a critical process regulating flowering time. The IWGSC RefSeq v1.0 (Appels et al. 2018) was used to design a CRISPR-Cas12a RNP targeting approach for one of these FLC genes, TaAGL33, present on all sub-genomes, A (TraesCS3A01G435000), B (TraesCS3B01G470000) and D (TraesCS3D01G428000). A synthetic Cas 12a crRNA composed of a 21 bp direct repeat sequence and a 23 bp protospacer was designed to target all three TaAGL33 alleles. The recognition site of the Cas 12a crRNA is located on the sense strand of TaAGL33 [5′-[(TTTC) AGCATAGAAGGTACATATGACCG-3′], with PAM site between brackets (FIG. 24).

Droplet Digital PCR (ddPCR) Assay to Identify Cas12a RNP-Induced FLC Indels

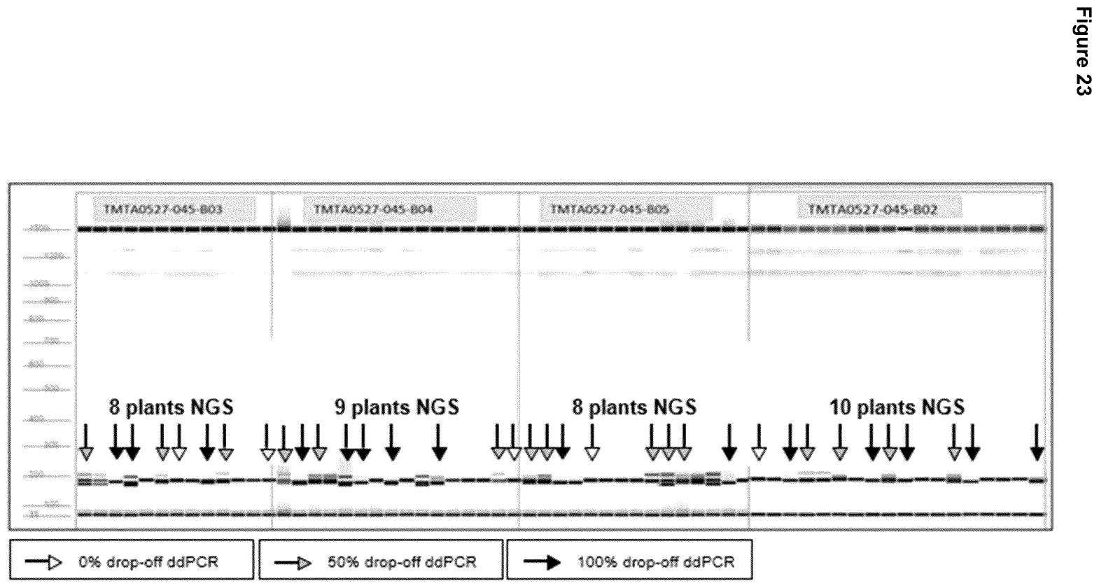

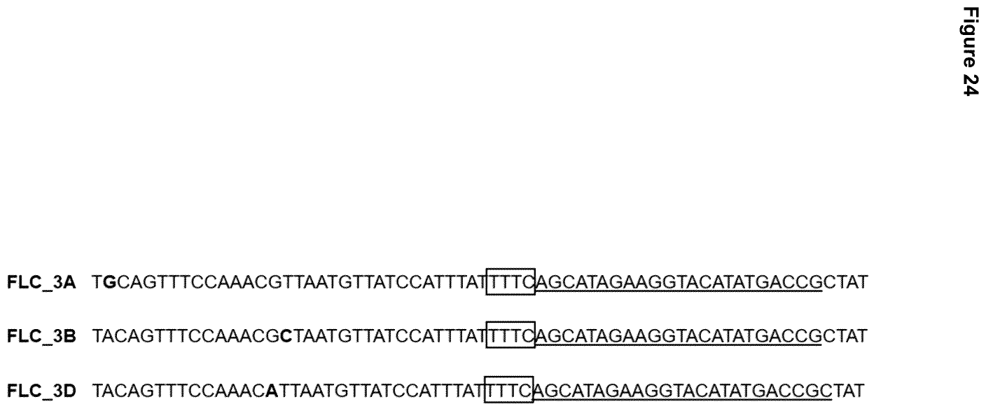

A ddPCR assay was designed and validated to identify Cas 12a-RNP induced FLC indels at the target site of cr-FLC-G1. The location of the predicted Cas 12a cut site in the FLC gene and the position of the PCR primers and probes used for the ddPCR analysis is shown in FIG. 25.

### Wheat Donor Plant Growth for Immature Embryo Isolation

Donor plants of cv. Fielder were grown under controlled-environment conditions to ensure optimal immature embryo quality. Typical growth conditions were day/night temperatures of 20° C.+/−1° C. and 18° C.+/−1° C., 65% relative humidity with a 16 h photoperiod (400 μmol/m²/s illumination at table level) provided by a mixture of 600W high pressure sodium lamps and 400W metal halide lamps.

### Preparation of Wheat Immature Embryos for Bombardment

Immature seeds were harvested from donor plants containing embryos around 2 mm in length, peeled and sterilized in 70% v/v ethanol for 1 min and followed by 10 min in 10% v/v sodium hypochlorite (ACROS bleach containing 10-15% active chlorine). Finally, the immature seeds were washed several times with demineralized water.

Embryos were aseptically excised from the immature seeds using a binocular microscope (Model MZ6, Leica) taking care to slice-off the embryo axis through the green seed coat during preparation. Subsequent steps for embryo preparation were performed using a modified procedure essentially as described by Ishida et al. (2015). Immature embryo explants were transferred to 3.5 cm Petri dishes (Falcon 351008) containing 4.5 ml of non-selective callus induction medium WLS, herewith designated callus induction medium 240. Embryos were arranged in a central circle of 1.5 cm (25-50 embryos/dish with the scutellum side facing upwards).

### Preparation of Cas12a RNP Complexes

Purified Cas 12a nuclease and FLC-specific crRNA were ordered from IDT (Integrated DNA Technologies) for RNP assembly.
crRNA: custom and user-defined crRNA that binds to 23 bases on the DNA strand that is opposite to the TTTV, PAM sequence
Cas12a nuclease: (L.b. Cas12a Ultra Nuclease): Recombinant Lachnospiraceae bacterium Cas12a nuclease, purified from E. coli strain expressing Cas 12a. Contains C-terminal nuclear localization signal (NLS) and C-terminal 6-His tag.

For RNP complex assembly, FLC-specific crRNA was mixed with Cas 12a nuclease in NEBuffer™ 2.1 (New England BioLabs) in an approximate equimolar ratio. The mixture was incubated at 37° C. for 20-30 min and then transferred to ice.

Biolistic Delivery of Cas12a RNP Complexes into Wheat

RNP delivery into immature embryos was conducted using the Biolistic@PDS-1000/He Particle Delivery System (Bio-Rad) using a modified procedure of Liang et al. (2018) adapted for Cas12a RNP delivery. RNP complexes were mixed with 0.6 μm gold particles (BioRad) together with TransIT-CRISPR® transfection reagent (Sigma-Aldrich), 15 μl aliquots were spread over the central region of each macrocarrier and airdried for 30 min in a laminar flow bench prior to delivery. Typically, for each shot 150 μg of gold particles and 3 μg of Cas 12a protein complexed with crRNA were delivered.

### Culture of Bombarded Wheat Immature Embryos and Plant Regeneration

Following bombardment, the immature embryos were incubated on the same plates for 48 h in the dark (28° C.+/−1° C., 55% relative humidity in an MLR-352H-PE Panasonic incubator). After this period embryos were transferred to fresh non-selective callus induction medium 240 (9 cm dishes containing 35-40 ml of medium with up to 15 embryos/dish), further incubated for 1 day with the same conditions and thereafter transferred to 25° C. (+/−1° C.).

One week after bombardment immature embryos were longitudinal bisected into 2 pieces under a binocular microscope and transferred to fresh non-selective callus induction medium 240 (9 cm dishes containing 35-40 ml of medium, 12 embryos/dish) and cultured in the dark (25° C.+/−1° C.).

After 2 weeks, the bisected immature embryos were once more bisected under a binocular microscope and transferred to fresh non-selective callus induction medium 240 (9 cm dishes containing 35-40 ml of medium, 4-6 embryos/dish) and cultured in the dark under the same conditions. After a further 2 weeks, the calli from each embryo were transferred intact to fresh nonselective callus induction medium 240 (9 cm dishes containing 35-40 ml of medium, 2 embryos/dish) and cultured in the dark under the same conditions.

Two weeks later, small pieces of structured embryogenic callus tissue were transferred to a non-selective regeneration medium, LSZ (Ishida et al., 2015), herewith designated regeneration medium 420. Regeneration medium 420 was prepared in PlantCon™™ containers (MP Biomedicals, Catalogue Nr. 26-722-06), 100 ml medium/container. The embryogenic calli arising from one embryo were transferred to one PlantCon™ container (max. 16 pieces/container). Where more than 16 embryogenic calli were recovered from one embryo, additional regeneration containers were utilized. PlantCon™ containers were incubated in the light for approximately 6 weeks (23° C.+/−1° C., 16 h photoperiod).

Shoots from PlantCon™ containers were transferred individually to De Wit tubes (Duchefa Biochemie) containing 10 ml of non-selective rooting medium WRM, essentially a modification of medium R (Sparks and Jones, 2009), solidified with 0.15% w/v Gelrite (Duchefa Biochemie) and cultured in the light (23° C.+/−1° C., 16 h photoperiod).

### Tissue Sampling and DNA Isolation

Sampling 1-Three days after bombardment, 5 immature embryos were randomly selected from Zone 2 (FIG. 5) of each bombarded plate. The embryos were bulked as one sample, collected in 2 ml tubes (Eppendorf® Safe-Lock) and stored at −80°. Samples were ground in a Retsch Mixer Mill MM300/400 for 60 sec. Genomic DNA extraction was performed using the Qiagen Dneasy Plant Mini Kit (Catalogue Nr. 69106) according to the manufacturer's instructions. Final DNA concentrations were measured, and plates stored at 4° C. until use for downstream analysis.

Sampling 2-Following 4-5 weeks of culture of structured embryogenic callus on non-selective regeneration medium 420 in PlantCon™ containers, leaf pieces were harvested from regenerating shoots. Care was taken to ensure that similar sized leaf pieces were taken from all regenerating shoots, this was best achieved by first cutting off all longer leaf tips with sterile scissors and then cutting leaf pieces of approximately 5 mm in length from the remaining tissues (FIG. 6). Leaf pieces originating from each PlantCon™ container were pooled and transferred to 6 ml screw-capped tubes (Micronic MP 32301) and stored at −80°. Samples were freeze dried overnight and genomic DNA extracted using a procedure based on the LGC GENOMICS sbeadex™ Maxi Plant Kit with KingFisher automation. Final DNA concentrations were measured, and plates stored at 4° C. until use for downstream analysis.

Sampling 3-Following 1-2 weeks of culture of individual shoots in De Wit tubes containing 10 ml of WRM medium, leaf pieces were harvested from shoots. Samples were collected in 1.4 ml push-cap tubes in a 96-sample carrier rack (Micronic MP 226RP) and stored at −80°. Genomic DNA was extracted using a procedure based on the LGC GENOMICS sbeadex™ Maxi Plant Kit with KingFisher automation. Final DNA concentrations were measured, and plates stored at 4° C. until use for downstream analysis.

### Selection/Analysis Process to Identify Cas12a RNP Indels in FLC

FIG. 7 shows an overview of the 3-step selection system used to identify RNP-induced indels in FLC in wheat plants. The process involves sequential enrichment for tissues carrying RNP-induced FLC indels through repeated sampling and molecular screening to specifically detect targeted mutations. In the example shown this was performed at 3 steps, in immature embryo explants, in regenerated populations of shoots and finally at the single plant level. A detailed overview of the selection/analysis process for Cas 12a RNP is shown in FIG. 17 and FIG. 18.

Three days after biolistic RNP delivery, 5 immature embryos from each shot were selected from the inner circle area (Zone 2) and sampled for DNA isolation and ddPCR analysis to determine FLC NHEJ drop-off percentages. A 20× assay mix was composed of 18 μM forward (Seq ID NO: 13) and 18 μM reverse (Seq ID NO: 14) primers, 5 M reference probe (Seq ID NO: 15) and 5 M drop-off probe (Seq ID NO: 16). The following reagents were mixed in a 96-well plate to make a 25-μl reaction: 11 μl of ddPCR Supermix for Probes (no dUTP, BioRad Laboratories), 1.1 μl of 20× assay mix, 100-250 ng of genomic DNA in water, and water up to 22 μl. The results are summarized in Table 14, ddPCR drop-off percentages ranged from 0.91% to 6.87%. Samples from shots 2, 3 and 4 showed the highest drop-off percentages and as such the remaining embryos from these shots were cultured further. The ddPCR results imply that these immature embryos are the ones most likely to carry cells with NHEJ indels in FLC alleles.

Immature embryos were passed-through 3 cycles of culture on non-selective callus induction medium 240 to obtain structured embryogenic callus from which plants could be regenerated on non-selective regeneration medium 420 in PlantCon™ containers. Leaf pieces originating from multiple shoots in each PlantCon™ container were pooled and transferred to 6 ml screw-capped tubes (Micronic MP 32301) for ddPCR analysis to determine FLC drop-off percentages. From the 3 cultured shots of TMTA0609 a total 156 pools were analyzed, originating from 79 individual embryos (59 pools from shot 2, 37 pools from shot 3 and 60 pools from shot 4). For ease of presentation the FLC drop-off percentages for each pool were classified into distinct categories (≤10%, >10-≤25%, >25-≤50% and >50%), results are shown in Table 15. As expected, shot 3, that showed the highest drop-off percentages at Sampling step 1 (immature embryos), exhibited higher drop-off values in the pooled-leaf samples, with approx. 30% of the pools having drop-off values of >25%.

Shoots from pools showing the highest drop-off percentages (10 pools from shot 2, 16 from shot 3 and 8 from shot 4) were selected for further culture. The ddPCR results imply that these pools are the ones most likely to carry shoots with NHEJ indels in FLC alleles.

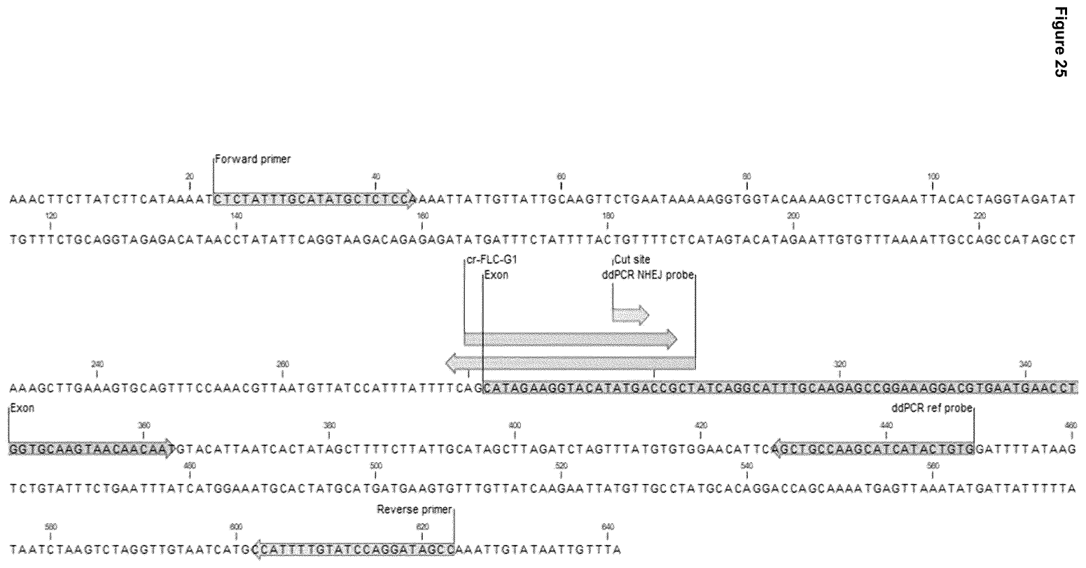

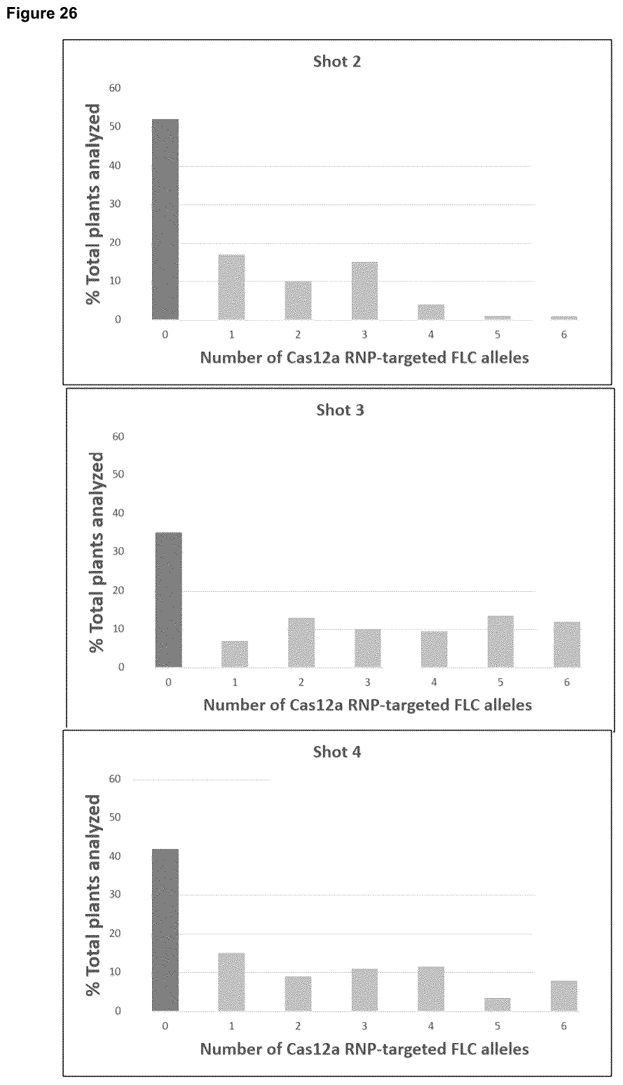

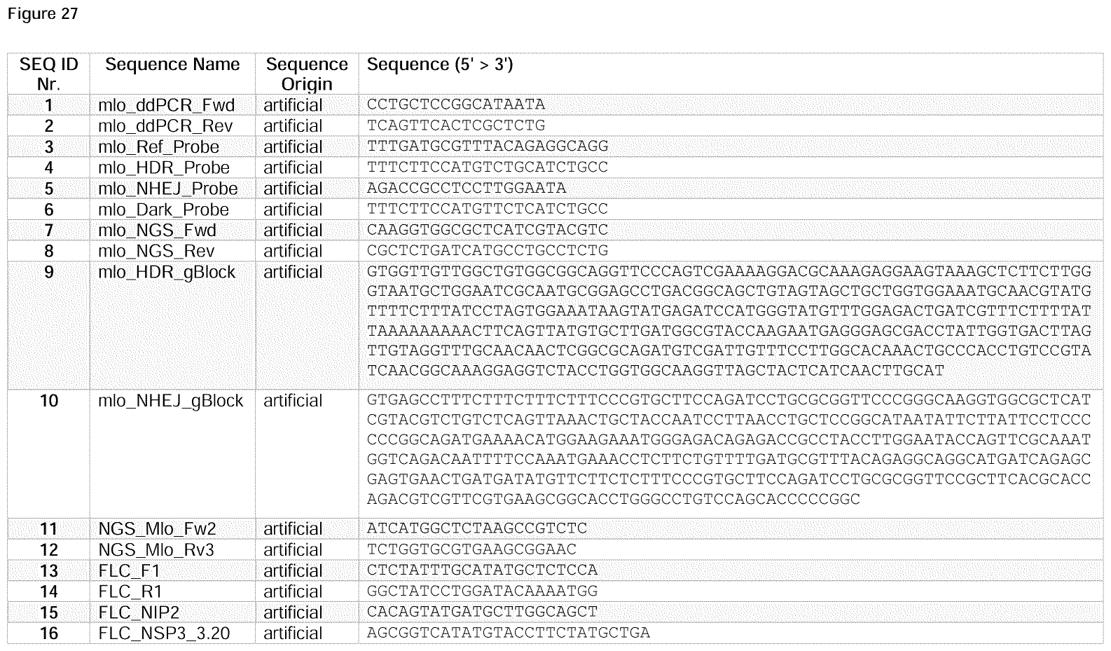

Shoots from selected containers were transferred to individual De Wit tubes containing 10 ml of non-selective rooting medium WRM for further development. Approximately 2 weeks after transfer, leaf samples from individual plants were taken for ddPCR analysis to determine mlo drop-off percentages. In total, samples were taken from 605 individual shoots originating from 32 immature embryo explants. Shoots were assigned to specific FLC targeting classes based on the drop-off percentages predicting the number of targeted alleles. Since the ddPCR assay can detect indels on all sub-genomic copies of FLC, the drop-off percentage can give a direct estimate of the number of alleles targeted in each plant; ˜17% drop-off=1 allele, ˜33% drop-off=2 alleles, ˜50% drop-off=3 alleles, ˜67% drop-off=4 alleles, ˜83% drop-off=5 alleles and 100%=all 6 alleles targeted. The results are shown for 589 of the shoots that could be clearly assigned to specific classes (Table 16). For the other 16 plants the ddPCR either failed or the plants could not be accurately assigned to any class. For shot 2, 189 plants were classified into classes (32 plants with one targeted allele, 18 plants with 2 targeted alleles, 28 plants with 3 targeted alleles, 8 plants with 4 targeted alleles, 2 plants with 5 targeted alleles, 2 plants with all 6 alleles targeted and 99 plants with no targeted alleles). Of the 138 plants analyzed from shot 4, 21 plants were identified with one targeted allele, 12 plants with 2 targeted alleles, 15 plants with 3 targeted alleles, 16 plants with 4 targeted alleles, 5 plants with 5 targeted alleles, 11 plants with all 6 alleles targeted and 58 with no targeted alleles). For shot 3, 262 plants were classified into classes (17 plants with one targeted allele, 35 plants with 2 targeted alleles, 27 plants with 3 targeted alleles, 25 plants with 4 targeted alleles, 35 plants with 5 targeted alleles, 32 plants with all 6 alleles targeted and 91 plants with no targeted alleles). Most mutant plants were identified from shot 3, which is to be expected given that the highest drop-off percentages were also observed in the 2 previous sampling steps (immature embryos and pooled leaf samples). Furthermore, the total number of FLC alleles targeted in each plant was also more prominent in shot 3 with higher levels of multi-allele targeting on the A, B and D copies of FLC (Figure 26). None of the FLC targeted plants were further characterized by NGS to accurately define indels at the sequence level. Consequently, the possibility exists that some plants recovered within the same pool and belonging to the same FLC-targeting class are clonal with identical mutation profiles.

### REFERENCES

Appels R, et al. Shifting the limits in wheat research and breeding using a fully annotated reference genome. Science, 2018, 361 (6403): 361-374.
Gil-Humanes J, Wang Y, Liang Z, Shan Q, Ozuna C V, Sánchez-León S, Baltes N J, Starker C, Barro F, Gao C, Voytas D F. High-efficiency gene targeting in hexaploid wheat using DNA replicons and CRISPR/Cas9. Plant J. 2017 March; 89 (6): 1251-1262.
Ishida Y, Tsunashima M, Hiei Y, Komari T. Wheat (Triticum aestivum L.) transformation using immature embryos. Methods Mol Biol. 2015; 1223:189-98.
Liang Z, Chen K, Zhang Y, Liu J, Yin K, Qiu J L, Gao C. Genome editing of bread wheat using biolistic delivery of CRISPR/Cas9 in vitro transcripts or ribonucleoproteins. Nat Protoc. 2018 March; 13 (3): 413-430.
Sparks C A, Jones H D. Biolistics transformation of wheat. In: Jones H D, Shewry P R, editors. Transgenic wheat, barley and oats: production and characterization protocols. Totowa: Humana Press; 2009. p. 71-92.

1. A method for the production of plants comprising a desired nucleic acid sequence from a population of plant cells comprising regenerative cells comprising a subpopulation of cells comprising the desired nucleic acid sequence, wherein the method comprises the steps of:
a) dividing a population of plant cells comprising regenerative cells, that comprises a subpopulation of cells comprising a desired nucleic acid sequence, into subgroups, quantifying the concentration of the desired nucleic acid sequence for each subgroup, each subgroup representing a subset of the populations genotype, and identifying one or more subgroups with the highest concentration of the desired nucleic acid sequence,
b) culturing cells from the one or more sub-group(s) with the highest concentration of the desired nucleic acid sequence, dividing the cells, into subgroups, quantifying the concentration of the desired nucleic acid sequence in each subgroup, each subgroup representing a subset of the population's genotype, and selecting one or more subgroups with the highest concentration of the desired nucleic acid sequence, and
c) regenerating intact individual plants from the cells of one or more selected subgroup(s) of step (c), with the desired nucleic acid sequence.

2. The method of claim 1, wherein the regenerative cells are selected from the group consisting of:
a. single cells,
b. cell aggregates,
c. complex multicellular explants from mature or immature seeds,
d. complex multicellular explants from seedlings, and
e. complex multicellular explants from plants.

3. The method of claim 1, wherein the population of plant cells comprising regenerative cells is first divided into subgroups and then, each subgroup is tested for the concentration of the desired nucleic acid sequence in said subgroup.

4. The method of claim 1, further comprising a step in which the nucleic acid molecules or genome of the plant cells are chemically mutated or ionized, gene or genome edited, or genetically engineered before the cells are divided into subgroups.

5. The method of claim 1, further comprising a step in which the nucleic acid molecules or genome of the plant cells are chemically mutated or ionized, gene or genome edited, or genetically engineered after the sub-groups of the regenerative cells have been formed.

6. The method of claim 1, wherein the cells, tissues or plants are cultured in a medium or growing condition which is not selective for the presence of a desired nucleic acid sequence in the genome of the regenerative cells.

7. The method of claim 1, further comprising a step for extracting the nucleic acid molecules from a sample of each subgroup of genetically modified cells and destroying the cells of the sample in the analysis of the concentration of the desired nucleic acid sequence in said sample.

8. The method of claim 1, comprising that the concentration of the nucleic acid sequence in the genome of the genetically modified plant cells is determined in a molecular screen.

9. The method of claim 1, comprising the following steps: (i) culturing cells from the selected subgroup(s) from step (a), (ii) extracting nucleic acid molecules from cells of one or more samples from each subgroup, (iii) identifying one or more subgroups with the highest concentrations of the desired nucleic acid sequence, and (iv) selecting one or more subgroups of said cells with the highest concentration of the desired nucleic acid sequence,

10. The method of claim 1, comprising the following steps: (i) recovering individual plants from the cells of the selected subgroup(s) from step (b), (ii) dividing the population of plants into subgroups, (ii) extracting DNA from one or more samples from said plants of each subgroup, (iii) pool the samples taken from individual plants from one subgroup and identifying one or more subgroups with the highest concentration(s) of the desired nucleic acid sequence, and (iv) selecting one or more subgroups of said plants with the highest concentration of the desired nucleic acid sequence.

11. A method for the production of a plant comprising a desired nucleic acid sequence comprising the steps:
(a1) (i) providing a population of plant cells comprising regenerative plant cells expected to have a desired nucleic acid sequence, (ii) dividing the population of the plant cells comprising regenerative plant cells into subgroups, (iii) extracting DNA from one or more samples from said cells of subgroups, (iv) identifying one or more subgroups with the highest concentration(s) of the desired nucleic acid sequence, and (v) selecting one or more subgroups of said cells with the highest concentration of the desired nucleic acid sequence,
whereby, optionally, cells are genetically modified cells,
or
(a2) (i) dividing the population plant cells comprising regenerative plant cells into subgroups, (ii) optionally, genetically modifying the subgroups of plant cells comprising regenerative plant cells, (iii) extracting DNA from one or more samples from said cells of each subgroup, (iv) identifying one or more subgroups with the highest concentration(s) of a desired nucleic acid sequence, and (v) selecting one or more subgroups of said cells with the highest concentration(s) of the desired nucleic acid sequence,
and
(b) (i) culturing the regenerative plant cells of the selected subgroup(s) of step (a1) or (a2), (ii) extracting DNA from one or more samples from the cultured plant cells of the subgroups, (iv) identifying one or more subgroups with the highest concentration(s) of the desired nucleic acid sequence, and (v) selecting one or more subgroups of said cultured cells with the highest concentration(s) of the desired nucleic acid sequence,
and,
(c1) (i) recovering individual plants or shoots from the cells of the selected subgroup(s) from step (b), (ii) dividing the population of plants or shoots into subgroups, (ii) extracting DNA and/or RNA from one or more samples from cells of each subgroup, (iii) identifying one or more subgroups with the highest concentration(s) of the desired nucleic acid sequence, and (iv) selecting one or more subgroups of said plants or shoots with the highest concentration(s) of the desired nucleic acid sequence and grow plants thereof,
or
(c2), (i) recovering individual plants or shoots from the cultured cells of the selected subgroup(s) from step (b), (ii) taking DNA and/or RNA comprising samples from each plant or shoot, (iii) analyzing said DNA and/or RNA for presence of the desired nucleic acid sequence, (iv) selecting the plants with the desired nucleic acid sequence and grow plants thereof,
whereby, optionally, the plant cells are genetically modified cells.

12. The method of claim 2, wherein:
a. the single cells are protoplasts or microspores,
b. the cell aggregates are cell suspensions or callus cultures,
c. complex multicellular explants from mature or immature seeds are immature embryos, scutella or cotyledons,
d. complex multicellular explants from seedlings are roots, hypocotyls, cotyledons, leaves, petioles or meristems, and
c. complex multicellular explants from plants are roots, leaves, leaf-bases, petioles, stems, or meristems.

13. The method of claim 8, wherein the molecular screen is next generation sequencing (NGS) or digital droplet PCR (ddPCR).


SEQUENCE LISTING (31 sequences)

SEQ ID NO: 1 - DNA, 17 bp, synthetic construct
  source 1..17: mol_type = other DNA
  misc_feature 1..17: note = mlo_ddPCR_Fwd
cctgctccggcataata

SEQ ID NO: 2 - DNA, 17 bp, synthetic construct
  source 1..17: mol_type = other DNA
  misc_feature 1..17: note = mlo_ddPCR_Rev
tcagttcactcgctctg

SEQ ID NO: 3 - DNA, 23 bp, synthetic construct
  source 1..23: mol_type = other DNA
  misc_feature 1..23: note = mlo_Ref_Probe
tttgatgcgtttacagaggcagg

SEQ ID NO: 4 - DNA, 23 bp, synthetic construct
  source 1..23: mol_type = other DNA
  misc_feature 1..23: note = mlo_HDR_Probe
tttcttccatgtctgcatctgcc

SEQ ID NO: 5 - DNA, 19 bp, synthetic construct
  source 1..19: mol_type = other DNA
  misc_feature 1..19: note = mlo_NHEJ_Probe
agaccgcctccttggaata

SEQ ID NO: 6 - DNA, 23 bp, synthetic construct
  source 1..23: mol_type = other DNA
  misc_feature 1..23: note = mlo_Dark_Probe
tttcttccatgttctcatctgcc

SEQ ID NO: 7 - DNA, 23 bp, synthetic construct
  source 1..23: mol_type = other DNA
  misc_feature 1..23: note = mlo_NGS_Fwd
caaggtggcgctcatcgtacgtc

SEQ ID NO: 8 - DNA, 23 bp, synthetic construct
  source 1..23: mol_type = other DNA
  misc_feature 1..23: note = mlo_NGS_Rev
cgctctgatcatgcctgcctctg

SEQ ID NO: 9 - DNA, 400 bp, synthetic construct
  source 1..400: mol_type = other DNA
  misc_feature 1..400: note = mlo_HDR_gBlock
gtggttgttggctgtggcggcaggttcccagtcgaaaaggacgcaaagaggaagtaaagctcttcttgggtaatgctggaatcgcaatgcggagcctgacggcagctgtagtagctgctggtggaaatgcaacgtatgttttctttatcctagtggaaataagtatgagatccatgggtatgtttggagactgatcgtttcttttattaaaaaaaaacttcagttatgtgcttgatggcgtaccaagaatgagggagcgacctattggtgacttagttgtaggtttgcaacaactcggcgcagatgtcgattgtttccttggcacaaactgcccacctgtccgtatcaacggcaaaggaggtctacctggtggcaaggttagctactcatcaacttgcat

SEQ ID NO: 10 - DNA, 392 bp, synthetic construct
  source 1..392: mol_type = other DNA
  misc_feature 1..392: note = mlo_NHEJ_gBlock
gtgagcctttctttctttctttcccgtgcttccagatcctgcgcggttcccgggcaaggtggcgctcatcgtacgtctgtctcagttaaactgctaccaatccttaacctgctccggcataatattcttattcctccccccggcagatgaaaacatggaagaaatgggagacagagaccgcctaccttggaataccagttcgcaaatggtcagacaattttccaaatgaaacctcttctgttttgatgcgtttacagaggcaggcatgatcagagcgagtgaactgatgatatgttcttctctttcccgtgcttccagatcctgcgcggttccgcttcacgcaccagacgtcgttcgtgaagcggcacctgggcctgtccagcacccccggc

SEQ ID NO: 11 - DNA, 21 bp, synthetic construct
  source 1..21: mol_type = other DNA
  misc_feature 1..21: note = NGS_Mlo_Fw2
atcatggctctaagccgtctc

SEQ ID NO: 12 - DNA, 20 bp, synthetic construct
  source 1..20: mol_type = other DNA
  misc_feature 1..20: note = NGS_Mlo_Rv3
tctggtgcgtgaagcggaac

SEQ ID NO: 13 - DNA, 22 bp, synthetic construct
  source 1..22: mol_type = other DNA
  misc_feature 1..22: note = FLC_F1
ctctatttgcatatgctctcca

SEQ ID NO: 14 - DNA, 22 bp, synthetic construct
  source 1..22: mol_type = other DNA
  misc_feature 1..22: note = FLC_R1
ggctatcctggatacaaaatgg

SEQ ID NO: 15 - DNA, 22 bp, synthetic construct
  source 1..22: mol_type = other DNA
  misc_feature 1..22: note = FLC_NIP2
cacagtatgatgcttggcagct

SEQ ID NO: 16 - DNA, 27 bp, synthetic construct
  source 1..27: mol_type = other DNA
  misc_feature 1..27: note = FLC_NSP3_3.20
agcggtcatatgtaccttctatgctga

SEQ ID NO: 17 - DNA, 60 bp, synthetic construct
  source 1..60: mol_type = other DNA
  misc_feature 1..60: note = Figure 1: MLO_4D: schematic sequence to indicate the position of the Cas9 gRNA in exon 4
atgaggacctggaagaaatgggagacagagaccgcctccctggaataccagttcgcaaat

SEQ ID NO: 18 - DNA, 65 bp, synthetic construct
  source 1..65: mol_type = other DNA
  misc_feature 1..65: note = Figure 15: MLO_5A: schematic sequence to indicate the position of the Cas12a gRNA in exon 4
atgagaacctggaagaaatgggagacagagaccgcctccctggaataccagttcgcaaatggtca

SEQ ID NO: 19 - DNA, 65 bp, synthetic construct; identical to SEQ ID NO: 18
  source 1..65: mol_type = other DNA
  misc_feature 1..65: note = Figure 15: MLO_4B: schematic sequence to indicate the position of the Cas12a gRNA in exon 4

SEQ ID NO: 20 - DNA, 65 bp, synthetic construct
  source 1..65: mol_type = other DNA
  misc_feature 1..65: note = Figure 15: MLO_4D: schematic sequence to indicate the position of the Cas12a gRNA in exon 4
atgaggacctggaagaaatgggagacagagaccgcctccctggaataccagttcgcaaatggtca

SEQ ID NO: 21 - DNA, 201 bp, synthetic construct
  source 1..201: mol_type = other DNA
  misc_feature 1..201: note = Figure 2: schematic sequence of the PCR design indicating the location, orientation and sequences of the different probes and primers for the Cas9 approach
tccttaacctgctccggcataatattcttattcctccccccggcagatgcagacatggaagaaatgggagacagagaccgcctccttggaataccagttcgcaaatggtcagacaattttccaaatgaaacctcttctgttttgatgcgtttacagaggcaggcatgatcagagcgagtgaactgatgatatgttcttctc

SEQ ID NO: 22 - DNA, 201 bp, synthetic construct; identical to SEQ ID NO: 21
  source 1..201: mol_type = other DNA
  misc_feature 1..201: note = Figure 16: schematic sequence of the PCR design indicating the location, orientation and sequences of the different probes and primers for the Cas12a approach

SEQ ID NO: 23 - DNA, 60 bp, synthetic construct
  source 1..60: mol_type = other DNA
  misc_feature 1..60: note = Figure 1: MLO_5A: schematic sequence to indicate the position of the Cas9 gRNA in exon 4
atgagaacctggaagaaatgggagacagagaccgcctccctggaataccagttcgcaaat

SEQ ID NO: 24 - DNA, 60 bp, synthetic construct; identical to SEQ ID NO: 23
  source 1..60: mol_type = other DNA
  misc_feature 1..60: note = Figure 1: MLO_4B: schematic sequence to indicate the position of the Cas9 gRNA in exon 4

SEQ ID NO: 25 - DNA, 64 bp, synthetic construct
  source 1..64: mol_type = other DNA
  misc_feature 1..64: note = Figure 24: FLC_3A: schematic sequence to indicate the position of the Cas12a gRNA in the 3 subgenomic copies of TaAGL33 (FLC)
tgcagtttccaaacgttaatgttatccatttattttcagcatagaaggtacatatgaccgctat

SEQ ID NO: 26 - DNA, 64 bp, synthetic construct
  source 1..64: mol_type = other DNA
  misc_feature 1..64: note = Figure 24: FLC_3B: schematic sequence to indicate the position of the Cas12a gRNA in the 3 subgenomic copies of TaAGL33 (FLC)
tacagtttccaaacgctaatgttatccatttattttcagcatagaaggtacatatgaccgctat

SEQ ID NO: 27 - DNA, 64 bp, synthetic construct
  source 1..64: mol_type = other DNA
  misc_feature 1..64: note = Figure 24: FLC_3D: schematic sequence to indicate the position of the Cas12a gRNA in the 3 subgenomic copies of TaAGL33 (FLC)
tacagtttccaaacattaatgttatccatttattttcagcatagaaggtacatatgaccgctat

SEQ ID NO: 28 - DNA, 641 bp, synthetic construct
  source 1..641: mol_type = other DNA
  misc_feature 1..641: note = Figure 25: schematic sequence of the PCR design indicating the location, orientation and sequences of the different probes and primers for the Cas12a (cr-FLC-G1) approach
aaacttcttatcttcataaaatctctatttgcatatgctctccaaaattattgttattgcaagttctgaataaaaaggtggtacaaaagcttctgaaattacactaggtagatattgtttctgcaggtagagacataacctatattcaggtaagacagagagatatgatttctattttactgttttctcatagtacatagaattgtgtttaaaattgccagccatagcctaaagcttgaaagtgcagtttccaaacgttaatgttatccatttattttcagcatagaaggtacatatgaccgctatcaggcatttgcaagagccggaaaggacgtgaatgaacctggtgcaagtaacaacaatgtacattaatcactatagcttttcttattgcatagcttagatctagtttatgtgtggaacattcagctgccaagcatcatactgtggattttataagtctgtatttctgaatttatcatggaaatgcactatgcatgatgaagtgtttgttatcaagaattatgttgcctatgcacaggaccagcaaaatgagttaaatatgattatttttataatctaagtctaggttgtaatcatgccattttgtatccaggatagccaaattgtataattgttta

SEQ ID NO: 29 - DNA, 23 bp, synthetic construct
  source 1..23: mol_type = other DNA
  misc_feature 1..23: note = Schematic sequence to indicate the recognition site of the crRNA on the antisense strand within exon 4 of mlo. PAM site pos 21 to 23
gaactggtattccaaggaggcgg

SEQ ID NO: 30 - DNA, 28 bp, synthetic construct
  source 1..28: mol_type = other DNA
  misc_feature 1..28: note = Schematic sequence to indicate the recognition site of the crRNA on the antisense strand within exon 4 of mlo. PAM site pos 1 to 4
tttgcgaactggtattccaaggaggcgg

SEQ ID NO: 31 - DNA, 27 bp, synthetic construct
  source 1..27: mol_type = other DNA
  misc_feature 1..27: note = Schematic sequence to indicate the recognition site of the crRNA on the sense strand of TaAGL33. PAM site pos 1 to 4
tttcagcatagaaggtacatatgaccg